(19) United States
(12) Patent Application Publication
(10) Pub. No.: US 2005/0130302 A1
(43) Pub. Date: Jun. 16, 2005

(54) Method and composition for regulating expansion of stem cells

(75) Inventors: Hiromitsu Nakauchi, Tokyo (JP); Atsushi Iwama, Tokyo (JP); Hideyuki Oguro, Tokyo (JP)

Correspondence Address: SEED INTELLECTUAL PROPERTY LAW GROUP PLLC, 701 FIFTH AVE, SUITE 6300, SEATTLE, WA, 98104-7092, US

(73) Assignee: ReproCELL Inc., Tokyo (JP)

(21) Appl. No.: 10/952,460

(22) Filed: Sep. 28, 2004

Related U.S. Application Data
(60) Provisional application No. 60/507,261, filed on Sep. 29, 2003.

(51) Int. Cl. C12N 5/08

(52) U.S. Cl. 435/372

(57) ABSTRACT

A method and substance for regulating expansion of a stem cell, such as a hematopoietic stem cell, is provided. Therefore, a method is provided for regulating expansion of a stem cell, such as a hematopoietic stem cell, a germ line stem cell, or a neural stem cell, comprising the steps of (A) providing, to the stem cell, Bmi-1 or a variant or fragment thereof and/or a Bmi-1 regulating agent in an amount sufficient for regulation of the expansion of the stem cell and (B) culturing the stem cell for a time sufficient for the regulation of expansion.

### BACKGROUND OF THE INVENTION

1. Field of the Invention

The present invention relates to the field of stem cells. More particularly, the present invention relates to a technique of regulating the expansion of tissue stem cells (e.g., neural stem cells, germ line stem cells, and hematopoietic stem cells) and to a therapy using the same. Even more particularly, the present invention relates to a composition, method, and kit for promotion of differentiation of stem cells or expansion of stem cells, or maintaining of pluripotency (or the level of undifferentiation) or self-replication capability. The present invention also relates to a stem cell (particularly, a hematopoietic stem cell, a neural stem cell, and a germ line stem cell) prepared by using such a method.

2. Description of the Related Art

Recently, attention has been focused on disease therapy using regeneration medicine (regeneration therapy). However, regeneration therapy has not yet reached a point where it is usually applied to a number of patients suffering from organ or tissue dysfunctions. To date, a very limited number of such patients have been treated by organ transplantation or use of an auxiliary medical system or apparatus. These therapies have problems in shortage of donors, rejection, infection, durability, and the like. In particular, donor shortage raises serious problems. In the case of bone marrow transplantation, bone marrow and umbilical cord blood banks have gradually become more widely used at home and abroad, though it is still difficult to provide a limited amount of samples to the number of patients in need. Therefore, there is an increasing demand for therapies using stem cells and regeneration medicine using the same in order to overcome the above-described problems.

The expansion and differentiation of hematopoietic stem cells are important for regeneration therapy. The appropriate appreciation of stem cells and their mechanisms is considered to play a key role in treating diseases. Recently, advances have been made in research on stem cells.

The principal outstanding problem is the identification of a factor which regulates, or particularly promotes, the expansion and differentiation of stem cells (e.g., hematopoietic stem cells, neural stem cells, and germ line stem cells).

Reconstruction of organs is crucial to regeneration medicine. Organ reconstruction is roughly divided into a method of constructing organs ex vivo and using the organs as artificial organs and a method of reconstructing organs in vivo. Even if stem cells are obtained, their applications are limited when a controllable regeneration method is not available.

On the other hand, gene therapy, in which a gene is introduced into stem cells which are in turn transplanted into a patient, has been tried. For example, it has been reported that a stem cell (CD34 positive bone marrow cell) having the IL-2 receptor γ chain introduced was transplanted into patients with X-linked severe combined immunodeficiency, and as a result, the clinical condition of the patients was improved (Cavazzana-Calvo, M. et al., Science, 288:669-672, 2000). However, there has been substantially no reported example that the expansion of a stem cell is regulated by introducing a gene thereinto.

Various proteins have an important role in regulating the expansion and differentiation of stem cells. For example, stem cell factor (SCF) (also known as steel factor) in hematopoietic stem cells has attracted attention.

SCF is produced by bone marrow stromal cells and acts on pluripotent stem cells, bone marrow cells, and lymphocyte precursor cells to support their expansion and differentiation. That is, it is believed that SCF acts on cells from hematopoietic stem cells to precursor cells so as to aid other cytokines which induce differentiation toward the final stage (S. Kitamura, Saitokain-no-Saizensen [Frontline of Cytokine], Yodo-sha, edited by T. Hirano, pp. 174-187, 2000).

However, the action of SCF alone seems to be weak, as it cannot work well unless it operates in cooperation with other factors. For example, SCF induces the differentiation and expansion of hematopoietic stem cells strongly in the presence of other cytokines, such as interleukin IL-3, IL-6, IL-11, granulocyte colony stimulating factor (G-CSF), or the like. SCF also induces the differentiation and expansion of mast cells, erythroblast precursor cells, granulocyte macrophage precursor cells, megakaryocyte precursor cells, and the like.

Therefore, it is considered that SCF does not directly control expansion and differentiation, but enhances the responsiveness of a number of kinds of hematopoietic cells to various cytokines while supporting the survival of the cells.

Thrombopoietin (TPO) has also attracted attention. This factor supports the differentiation of megakaryocytes and the production of platelets as well as acting on stem cells to induce their expansion and differentiation. Also, it has been found that TPO is involved in the self replication of stem cells.

Thus, conventional factors can promote the differentiation of stem cells in an uncontrollable manner, but not in a controllable manner.

Most recently, attention has been focused on a factor called Bmi-1. Studies on the properties of this factor have just begun. The effect of external application of the factor is not known.

### SUMMARY OF THE INVENTION

The above-described problems have been solved by the present inventors who discovered the unexpected fact that Bmi-1 is capable of promoting of the expansion of stem cells, such as hematopoietic stem cells, neural stem cells, and germ line stem cells.

The Polycomb group (PcG) gene Bmi-1 has recently been implicated in the maintenance of hematopoietic stem cells (HSC) using loss-of-function analysis. Here we demonstrate that increased expression of Bmi-1 promotes HSC self-renewal. Forced (induced) expression of Bmi-1 enhanced symmetrical cell division of HSCs and mediated a higher probability of inheritance of stem cell characteristics through cell division. Correspondingly, forced expression of Bmi-1 but not the other PcG genes led to a striking ex vivo expansion of multipotential progenitors and marked augmentation of HSC repopulating capacity in vivo. Loss-of-function analyses revealed that among PcG genes, absence of Bmi-1 is preferentially linked with a profound defect in HSC self-renewal. Our findings define Bmi-1 as a central player in HSC self-renewal and demonstrate that Bmi-1 is a novel target for therapeutic manipulation of HSCs.

In the present invention, both loss-of-function and gain-of-function analysis revealed a central role for Bmi-1, but not other components, in the maintenance of HSC self-renewal both in vitro and in vivo, and in augmentation of HSC activity ex vivo. Our findings indicate that the expression level of Bmi-1 is the critical determinant for the self-renewal capacity of HSC.

According to an aspect of the present invention, a method for regulating expansion of a stem cell is provided. The method comprises the steps of: (A) providing, to the stem cell, Bmi-1 or a variant or fragment thereof and/or a Bmi-1 regulating agent in an amount sufficient for regulation of the expansion of the stem cell; and (B) culturing the stem cell for a time sufficient for the regulation of the expansion.

In one embodiment of this invention, the regulation of the expansion is the promotion of the expansion.

In one embodiment of this invention, the Bmi-1 or a variant or fragment thereof and/or the Bmi-1 regulating agent are exogenous or endogenous.

In one embodiment of this invention, the Bmi-1 or a variant or fragment thereof and/or the Bmi-1 regulating agent are exogenous.

In one embodiment of this invention, the Bmi-1 or a variant or fragment thereof and/or the Bmi-1 regulating agent is in the form of a nucleic acid and/or a protein.

In one embodiment of this invention, the Bmi-1 or a variant or fragment thereof and/or the Bmi-1 regulating agent include: (a) a polypeptide encoded by a nucleic acid sequence as set forth in SEQ ID NO: 1 or 3 (Accession No. L13689 or M64279, respectively) or a fragment thereof; (b) a polypeptide having an amino acid sequence as set forth in SEQ ID NO:2 or 4 or a fragment thereof; (O) a variant polypeptide having an amino acid sequence as set forth in SEQ ID NO:2 or 4 having at least one amino acid mutation selected from the group consisting of substitutions, additions, and deletions, the variant polypeptide having a biological activity; or (d) a polypeptide having at least 70% amino acid sequence homology to any one of polypeptides (a) to (c) and having biological activity.

In one embodiment of this invention, the Bmi-1 or a variant or fragment thereof and/or the Bmi-1 regulating agent include: (a) a polynucleotide having a base sequence as set forth in SEQ ID NO:1 or 3 (Accession No. L13689 or M64279, respectively) or a fragment thereof; (b) a polynucleotide encoding an amino acid sequence as set forth in SEQ ID NO:2 or 4 or a fragment thereof; (c) a polynucleotide encoding a variant polypeptide having an amino acid sequence as set forth in SEQ ID NO:2 or 4 having at least one amino acid mutation consisting of substitutions, additions, and deletions, and having biological activity; (d) a polynucleotide encoding a polypeptide hybridizable to any one of the polynucleotides of (a) to (c) under stringent conditions; or (e) a polynucleotide encoding a polypeptide having a base sequence having at least 70% identity to any one of the polynucleotides of (a) to (c) or a complementary sequence thereof and having biological activity.

In one embodiment of this invention, the Bmi-1 or a variant or fragment thereof and/or the Bmi-1 regulating agent are selected from the group consisting of a small molecule, a lipid molecule, a sugar, and a complex thereof.

In one embodiment of this invention, the stem cell includes a stem cell selected from the group consisting of hematopoietic stem cells, germ line stem cells, and neural stem cells.

According to another aspect of the present invention, a method is provided for regulating a disease, disorder, or abnormality related to hematopoiesis, reproduction, or nervous system, comprising the steps of: (A) providing, to a subject, Bmi-1 or a variant or fragment thereof and/or a Bmi-1 regulating agent in an amount sufficient for regulation of the expansion of a stem cell; and (B) allowing the subject sufficient time for regulation of expansion to occur.

According to another aspect of the present invention, a composition for regulating expansion of a stem cell is provided. The composition comprises Bmi-1 or a variant or fragment thereof and/or a Bmi-1 regulating agent in an amount sufficient for regulation of the expansion.

In one embodiment of this invention, the regulation of the expansion is the promotion of the expansion.

In one embodiment of this invention, the stem cell includes a stem cell selected from the group consisting of hematopoietic stem cells, germ line stem cells, and neural stem cells.

In one embodiment of this invention, the Bmi-1 or a variant or fragment thereof and/or the Bmi-1 regulating agent include: (a) a polypeptide encoded by a nucleic acid sequence as set forth in SEQ ID NO:1 or 3 (Accession No. L13689 or M64279, respectively) or a fragment thereof; (b) a polypeptide having an amino acid sequence as set forth in SEQ ID NO:2 or 4 or a fragment thereof; (c) a variant polypeptide having an amino acid sequence as set forth in SEQ ID NO:2 or 4 having at least one amino acid mutation selected from the group consisting of substitutions, additions, and deletions, the variant polypeptide having a biological activity; or (d) a polypeptide having at least 70% amino acid sequence homology to any one of polypeptides (a) to (c) and having biological activity.

In one embodiment of this invention, the Bmi-1 or a variant or fragment thereof and/or the Bmi-1 regulating agent include: (a) a polynucleotide having a base sequence as set forth in SEQ ID NO:1 or 3 (Accession No L13689 or M64279, respectively) or a fragment thereof; (b) a polynucleotide encoding an amino acid sequence as set forth in SEQ ID NO:2 or 4 or a fragment thereof; (c) a polynucleotide encoding a variant polypeptide having an amino acid sequence as set forth in SEQ ID NO:2 or 4 having at least one amino acid mutation consisting of substitutions, additions, and deletions, and having biological activity; (d) a polynucleotide encoding a polypeptide hybridizable to any one of the polynucleotides of (a) to (c) under stringent conditions; or (e) a polynucleotide encoding a polypeptide having a base sequence having at least 70% identity to any one of the polynucleotides of (a) to (c) or a complementary sequence thereof and having biological activity.

In one embodiment of this invention, the Bmi-1 complexes with Mph-1/Rae28 or M33.

In one embodiment of this invention, the Bmi-1 complexes with Mph-1/Rae28 and M33.

In one embodiment of this invention, the Bmi-1 or a fragment or variant thereof and/or the Bmi-1 regulating agent is consistently active.

In one embodiment of this invention, the Bmi-1 or a fragment or variant thereof and/or the Bmi-1 regulating agent is transiently active.

In one embodiment of this inventions the Bmi-1 consists of a sequence as set forth in SEQ ID NO:1 (Accession No. L13689).

In one embodiment of this invention, the composition further comprises a cellularly physiologically active substance.

In one embodiment of this invention, the cellularly physiologically active substance comprises an agent selected from the group consisting of SCF, TPO, and Flt-3L.

In one embodiment of this invention, the composition further comprises a pharmaceutically acceptable carrier.

In one embodiment of this invention, the Bmi-1 regulating agent comprises an agent capable of activating Bmi-1.

In one embodiment of this invention, the composition further comprises an agent capable of binding to an agent capable of increasing an activity of a promoter of Bmi-1 or a PcG complex comprising Bmi-1, to enhance a function thereof.

In one embodiment of this invention, the Bmi-1 or a fragment or variant thereof and/or the Bmi-1 regulating agent is in the form of a protein or a complexed protein.

In one embodiment of this invention, the Bmi-1 or a fragment or variant thereof and/or the Bmi-1 regulating agent is in the form of a nucleic acid.

In one embodiment of this invention, the Bmi-1 or a fragment or variant thereof and/or the Bmi-1 regulating agent in the form of a nucleic acid is contained in a vector.

In one embodiment of this invention, the vector is a retrovirus vector.

According to another aspect of the present invention, a method for treatment or prophylaxis of a disease, disorder, or abnormality related to hematopoiesis, reproduction, and a nervous system, is provided. The method comprises the step of: administering Bmi-1 or a variant or fragment thereof and/or a Bmi-1 regulating agent in an amount sufficient for the treatment or prophylaxis of a subject requiring regulation thereof.

According to another aspect of the present invention, a pharmaceutical composition for treatment or prophylaxis of a disease, disorder, or abnormality related to hematopoiesis, reproduction, and the nervous system, is provided. The pharmaceutical composition comprises: Bmi-1 or a variant or fragment thereof and/or a Bmi-1 regulating agent in an amount sufficient for treatment or prophylaxis.

According to another aspect of the present invention, a kit for regulating expansion of a stem cell is provided. The kit comprises: (A) a composition comprising Bmi-1 or a variant or fragment thereof and/or a Bmi-1 regulating agent in an amount sufficient for regulation of the expansion; and (B) instructions setting forth a method of providing the composition to the stem cell and, culturing the stem cell.

According to another aspect of the present invention, use of Bmi-1 or a variant or fragment thereof and/or a Bmi-1 regulating agent for regulating expansion of a stem cell is provided.

According to another aspect of the present invention, a method for producing a cell expanded from a stem cell line is provided. The method comprises the steps of: (A) providing a stem cell or a primordial cell; (B) providing, to the stem cell or the primordial cell, Bmi-1 or a variant or fragment thereof and/or a Bmi-1 regulating agent in an amount sufficient for regulation of expansion thereof; and (C) culturing the stem cell for a time sufficient for the regulation of the expansion.

In one embodiment of this invention, the stem cell includes a stem cell selected from the group consisting of hematopoietic stem cells, germ line stem cells, and neural stem cells.

According to another aspect of the present invention, a cell obtained by the above-described method is provided.

According to another aspect of the present invention, a tissue obtained by the above-described method is provided.

According to another aspect of the present invention, an organ obtained by the above-described method is provided.

According to another aspect of the present invention, a medicament comprising a cell obtained by the above-described method is provided.

According to another aspect of the present invention, a method for treatment or prophylaxis of a disease or disorder requiring a stem cell or an expansion cell derived therefrom is provided. The method comprises the step of: (A) administering a cell obtained by the above-described method to a subject requiring treatment or prophylaxis.

According to another aspect of the present invention, use of a cell obtained by the above-described method is provided for treatment or prophylaxis of a disease or disorder requiring a stem cell or an expansion cell derived therefrom.

According to another aspect of the present invention, a method for screening for an agent for regulating expansion of a stem cell is provided. The method comprises the steps of: (A) providing candidate substances for the agent; (B) exposing a cell containing Bmi-1 to the substances; and (C) determining whether or not the Bmi-1 is regulated. When the Bmi-1 is regulated, the substance is determined to be an agent capable of regulating the expansion of the stem cell.

According to another aspect of the present invention, an agent for regulating expansion of a stem cell, obtained by the above-described method, is provided.

These and other advantages of the present invention will become apparent to those skilled in the art upon reading and understanding the following detailed description with reference to the accompanying figures.

### BRIEF DESCRIPTION OF THE DRAWINGS

FIG. 1 is a schematic diagram showing Bmi-1 (lower panel). FIG. 1 also shows the expression of Bmi-1 in mouse hematopoietic stem cells.

FIG. 2 is a diagram showing the effect of Bmi-1 transduction on cell proliferation.

FIG. 3 is a diagram showing the differentiation of colony forming unit-granulocyte/erythrocyte/macrophage/megakaryocyte (CFU-GEMM) by Bmi-1 (Day 9).

FIG. 4 is a diagram showing the differentiation of colony forming unit-granulocyte/erythrocyte/macrophage/megakaryocyte (CFU-GEMM) by Bmi-1 (Day 14).

FIG. 5 is a diagram showing the results of Example 2 on Day 7 of culture.

FIG. 6 is a diagram showing the results of Example 2 on Day 14 of culture.

FIG. 7 is a diagram showing the expression of Bmi-1 (the chimeric phenomenon of donor cells in the peripheral blood) after transplantation into an animal.

FIG. 8 is a schematic diagram showing a differentiation scheme.

FIG. 9A shows the results of immunological staining of Bmi-1.

FIG. 9B shows the results of western blotting of Bmi-1 using the spleen from the same individual from which the testis was removed.

FIG. 10A shows the testis sections of 8-week-old wild type mice (WT; a, b), heteromice (+/−; c, d), and knockout mice (−/−; e, f).

FIG. 10B shows the results of HE staining of 18-week-old wild type mice (a, b) and 22-week-old wild type mice (c, d), and 18-week-old Bmi-1 knockout mice (−/−; e, f).

FIG. 11 shows ther role played by components of the Bmi-1-containing complex in HSC. (a) mRNA expression of mouse PcG genes in hematopoietic cells. Cells analyzed are bone marrow CD34⁻c-Kit⁺Sca-1⁺Lineage marker—stem cells (CD34⁻KSL), CD34³⁰ KSL progenitors, Lineage marker—cells (Lin⁻), Gr-1⁺ neutrophils, Mac-1⁺ monocytes/macrophages, TER119+erythroblasts, B220⁺B cells, spleen Thy-1.2⁺T cells, NK1.1⁺ NK cells, B220⁺ B cells, and thymic CD4⁻CD8⁻ T cells (DN), CD4+ CD8+ T cells (DP), CD4⁺CD8⁻ T cells (CD4SP), and CD4−CD8+ (CD8SP). (b) Competitive lymphohematopoietic repopulating capacity of PcG gene-deficient HSCs. The indicated number of E14 fetal liver cells from Bmi-1^{-/-}, Mel-18^{-/-}, and M33^{-/-} mice (B6-Ly5.2) and B6-Ly5.1 competitor cells were mixed and injected into lethally irradiated B6-Ly5.1 recipient mice. Percent chimerism of donor cells 4 and 12 weeks after transplantation is presented as mean±S.D.

FIG. 12 shows defective self-renewal and accelerated differentiation of Bmi-1^{-/-} HSCs. (a) Growth of Bmi-1^{-/-} CD34−KSL HSCs in vitro. Freshly isolated CD34'KSL cells were cultured in the presence of SCF and TPO for 14 days. The results are shown as mean±S.D. of triplicate cultures. (b) Single cell growth assay. Ninty-six individual CD34−KSL HSCs were sorted clonally into 96 well micro-titer plates in the presence of SCF, IL-3, TPO, and EPO. The numbers of high and low proliferative potential-colony-forming cells (HPP-CFC and LPP-CFC) were retrospectively evaluated by counting colonies at day 14 (HPP-CFC and LPP-CFC: colony diameter >1 mm and <1 mm, respectively). The results are shown as mean±S.D. of triplicate cultures. (c) Frequency of each colony type. Colonies derived from HPP-CFC were recovered and morphologically examined for the composition of colony-forming cells. (d) Paired daughter assay. When a single CD34⁻KSL rSC underwent cell division and gave rise to two daughter cells, daughter cells were separated by micromanipulation and were further cultured to permit full differentiation along the myeloid lineage. The colonies were recovered for morphological examination.

FIG. 13 shows rescue of defective HSC function in Bmi-1^{-/-} cells by re-expression of Bmi-1. CD34⁻KSL cells transduced with indicated retroviruses (GFP control, Bmi-1, and Bcl-xl) were plated in methylcellulose medium to allow colony formation 36 hr after the initiation of transduction. GFP+ colonies larger than 1 mm in diameter, which were derived from HPP-CFCs, were counted at day 14 (a), and recovered for morphological analysis to evaluate frequency of each colony type (b). The results are shown as mean±S.D. of triplicate cultures. (c) Indicated numbers of Bmi-1^{-/-} CD34⁻KSL cells were transduced with Bmi-1. After 3.5 days from the initiation of transduction, cells were injected into lethally irradiated Ly5.1 recipient mice along with Ly5.1 competitor cells. Repopulation by rescued Bmi-1^{-/-} CD34⁻KSL cells was evaluated by monitoring donor cell chimerism in peripheral blood 12 weeks after transplantation.

FIG. 14 shows ex vivo expansion of CFU-nmEM by forced (induced) expression of Bmi-1 in HSCs. (a) CD34⁻KSL cells transduced with the indicated PcG gene retroviruses were cultured in the presence of SCF and TPO and their growth was monitored. Morphology of cultured cells at 14 days was observed under an inverted microscope (inset). (b) At 14 days of culture, colony assays were performed to evaluate the content of HPP-CFC in culture. GFP+ colonies derived from HPP-CFCs were examined on their colony types by morphological analysis. (c) Net expansion of CFU-nmEM during the 14-day culture period. The results are shown as mean±S.D. of triplicate cultures.

FIG. 15 show forced expression of Bmi-1 promotes symmetrical cell division of HSCs. CD34⁻KSL HSCs were transduced with either GFP or Bmi-1 retroviruses. After 24 hr following transduction, cells were separated clonally by micromanipulation when a single cell underwent cell division, daughter cells were separated again by micromanipulation and were further cultured to permit full differentiation along the myeloid lineage. The colonies were recovered for morphological examination. Only the pairs whose parental cells should have retained neutrophil (a), macrophage (m), erythroblast (E), and Megakaryocyte (M) differentiation potential were selected. The probability of symmetrical cell division of daughter cells transduced with Bmi-1 was significantly higher than the control (p<0.044).

FIG. 16 shows enhancement of repopulation activity of HSCs by Bmi-1 expression. CD34⁻KSL cells either from wild type or p19^{-/-} mice were transduced with indicated retroviruses, and were further cultured in the presence of SCF and TPO. Competitive repopulation assays were performed using cultured cells at day 10 corresponding to 20 initial CD34⁻KSL cells per recipient mouse. Percent chimerism of donor cells, 12 weeks after transplantation, is plotted as dots and their mean values are indicated as bars (a). Percent chimerism in each lineage and repopulation units (RU) of each population (b). *p<0.001. RT-PCR analysis was performed on the wild type CD34⁻KSL cells that were transduced with the indicated retrovirus and cultured for 14 days in the presence of SCF and TPO (c).

FIG. 17 shows a radioprotection assay and analysis of Bmi-1^{-/-} microenvironmento to support hematopoiesis. (a) Radioprotective capacity of Bmi-1^{-/-} hematopoietic cells. Fetal liver cells (2×10⁶) from E14.5 embryos and total BM cells from 4-wk-old mice were transplanted into lethally irradiated B6-ly5.1 mice. Radioprotective capacity of the transplanted cells was evaluated by monitoring survival of recipient mice. (b) Ability of Bmi-1^{-/-} microenvironment to support hematopoiesis. BM cells from B6-Ly5.1 mice were transplanted to 8-wk-old Bmi-1^{-/-} mice (B6-ly5.2) irradiated at a dose of 6.0 Gy. After 8 wk, percent chimerism of donor cells in peripheral blood was analyzed. Then, BM and spleen cells recovered from one of the recipients were transplanted into secondary recipients (B6-Ly5.2) irradiated at a dose of 9.5 Gy. After 14 wk, percent chimerism of donor cells in peripheral blood was obtained. The results are shown as mean+/−S.D.

FIG. 18 shows the development of myeloid progenitors in vivo. Committed myeloid progenitors were detected as lL-7Rα⁻Lin⁻Sca⁻c-Kit⁺CD34⁺FcgRII/III^{lo} (CMP), lL-7Rα⁻Lin⁻Sca⁻c-Kit⁺CD34⁺FcgRII/III^{hi} (GMp), and lL-7Rα⁻Lin⁻Sca⁻c-Kit⁺CD34⁻FcgRII/III^{lo} (MEP) on a FACS Vantage as previously described (Akashi, K., Traver, D., Miyamoto, T., & Weissman, IL. A clonogenic common myeloid progenitor that gives rise to all myeioid lineages. Nature 404, 193-197, 2000). Numbers indicate frequency of each myeloid progenitor in the c-Kit⁺Sca⁻Lin⁻ population.

FIG. 19 shows effect of de-repression of p16^{Ink4a} and p19^{Arf} gene expression in HSC. (a) mRNA expression of mouse p16 and p19 genes in freshly isolated normal hematopoietic cells and in Bmi-1^{-/-} Lin⁻ cells. (b) Cell cycle status of BM KSL cells from 4-wk-old mice. Cell cycle status of KSL cells was measured using Hoechst 33342 DNA Dye (Sigma) on a FACS Vantage. The results are shown as mean+/−S. D. (n=4). (c) Cumulative percentage of first cell division of CD34KSL cells. Ninety-six individual CD34KSL HSCs were sorted clonally into 96 well micro-titer plates containing SCF and TPO. The day on which the first cell division occurred was microscopically monitored for each single cell. (d) Frequency of apoptotic cells in BM. Apoptosis was evaluated using BM cells from 6 to 8-wk-old mice using propidium iodide and anti-Annexin V antibody (PharMingen) on a FACS Vantage. Proportion of early (Annexin V⁺PI⁻), late (Annexin V⁺PI⁺), and total apoptotic cells (early plus late) are indicated. The results are shown as mean+/−S.D. (n=7).

### DESCRIPTION OF SEQUENCE LISTING

SEQ ID NO:1 is the nucleic acid sequence of human Bmi-1 (Accession No. L13689).

SEQ ID NO:2 is the amino acid sequence of human Bmi-1.

SEQ ID NO:3 is the nucleic acid sequence of mouse Bmi-1 (Accession No. M64279).

SEQ ID NO:4 is the amino acid sequence of mouse Bmi-1.

SEQ ID NO:5 is the nucleic acid sequence of human Mel-18 (Accession No. NM_{—}007144).

SEQ ID NO: 6 is the amino acid sequence of human Mel-181.

SEQ ID NO:7 is the nucleic acid sequence of mouse Mel-18 (Accession No. D90085).

SEQ ID NO: 8 is the amino acid sequence of mouse Mel-18.

SEQ ID NO:9 is the nucleic acid sequence of human Mph-1 (Accession No. NM_{—}004426).

SEQ ID NO:10 is the amino acid sequence of human Mph-1.

SEQ ID NO:11 is the nucleic acid sequence of mouse Mph-1 (Accession No. U63386).

SEQ ID NO:12 is the amino acid sequence of mouse Mph-1.

SEQ ID NO:13 is the nucleic acid sequence of human M33 (Accession No. XP_{—}300674).

SEQ ID NO:14 is the amino acid sequence of human M33.

SEQ ID NO:15 is the nucleic acid sequence of mouse M33 (Accession No. BC035199).

SEQ ID NO:16 is the amino acid sequence of mouse M33.

SEQ ID NO:17 is a nucleic acid sequence encoding GFP (Accession No. AJ249646).

SEQ ID NO:18 is the amino acid sequence of GFP.

SEQ ID NO:19 is a nucleic acid sequence encoding a RING finger domain deletion (DRF; D18-56) mutant of Bmi-1 (SEQ ID NO:4).

SEQ ID NO:20 is the amino acid sequence of the RING finger domain deletion (DRF: D18-56) mutant of Bmi-1 (SEQ ID NO:4).

SEQ ID NO:21 is a nucleic acid sequence encoding a HTHTHT domain deletion (DHT; D165-220) mutant of Bmi-1 (SEQ ID NO:4).

SEQ ID NO:22 is the amino acid sequence of the HTHTHT domain deletion (DHT; D165-220) mutant of Emi-1 (SEQ ID NO:4).

SEQ ID NO:23 is a nucleic acid sequence encoding a proline/serine-rich region deletion (DP/S: D248-324) mutant of Bmi-1 (SEQ ID NO:4).

SEQ ID NO:24 is the amino acid sequence of the proline/serine-rich region deletion (DP/S; D248-324) mutant of Bmi-1 (SEQ ID NO:4).

SEQ ID NO:25 is a nucleic acid sequence encoding a Hox gene (4B) SEQ ID NO:26 is the amino acid sequence of the Hox gene (4B).

SEQ ID NO:27 is a nucleic acid sequence encoding a p16^{INK4a}.

SEQ ID NO:28 is the amino acid sequence of the p16^{INK4a}.

SEQ ID NO:29 is a nucleic acid sequence encoding a p19^{ARF}.

SEQ ID NO: 30 is the amino acid sequence of the p19^{ARF}.

### DESCRIPTION OF THE PREFERRED EMBODIMENTS

It should be understood throughout the present specification that expression of a singular form includes the concept of their plurality unless otherwise mentioned. Specifically, articles for a singular form (e.g., “a”, “an”, “the”, etc. in English) include the concept of their plurality unless otherwise mentioned. It should be also understood that the terms as used herein have definitions typically used in the art unless otherwise mentioned. Thus, unless otherwise defined, all scientific and technical terms have the same meanings as those generally used by those skilled in the art to which the present invention pertains. If there is contradiction, the present specification (including the definition) takes precedence.

(Definitions)

Terms particularly used herein are defined as follows.

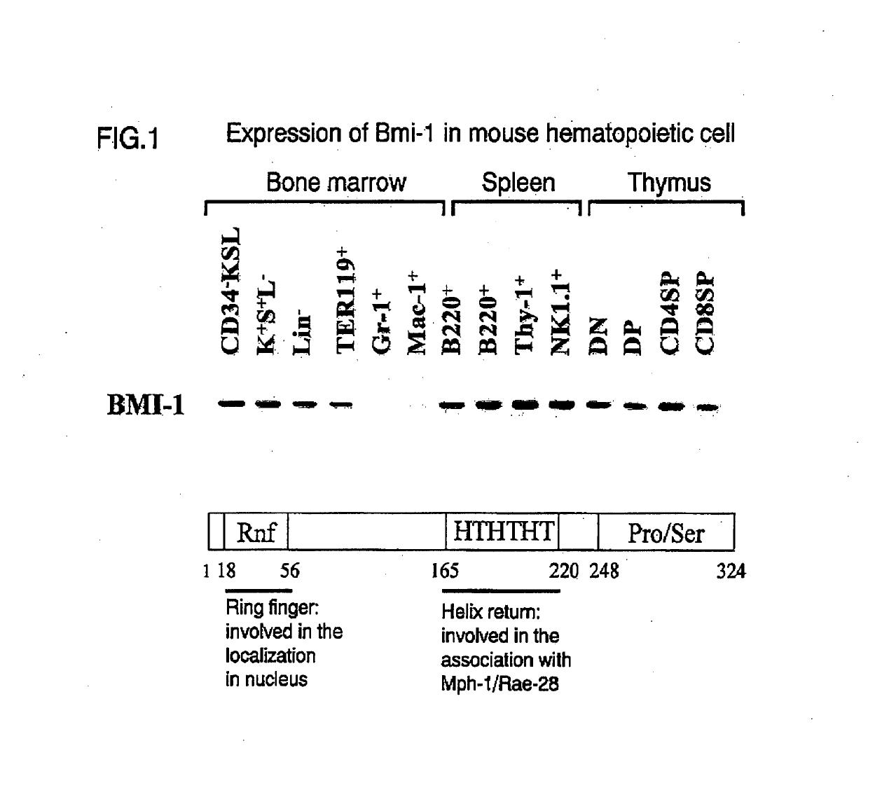

As used herein, “Bmi-1” refers to a factor which has been identified as a type of oncogene, i.e., a member of the Polycomb Group (PcG) family. Representative examples of Bmi-include, but are not limited to, agents having nucleic acid sequences as set forth in SEQ ID NOs:1 and 3 (Accession Nos. L13689 (human) and M64279 (mouse), respectively (nucleic acid sequences)) and SEQ ID NOs:2 and 4 (human and mouse, respectively (amino acid sequences)) and corresponding agents (orthologs) of other animal species. Bmi-1 has a nuclear localized sequence called ring finger (Rnf) at the N terminus thereof, for example, as shown in FIG. 1. Such a sequence has a characteristic sequence, such as, representatively C-X-(I, V)-C-X₁₁₋₃₀-C-X-H-X-(F,I,L)-C-X₂-C-(I,L,M)-X₁₀₋₁₉-C-P-X-C. In addition, Bmi-1 has a helix return portion representatively indicated by HTHTHT at substantially the middle portion thereof (approximately position 165-220 in the sequence as set forth in SEQ ID NOs:2 and 4 of FIG. 1). Bmi-1 is characterized in that the helix return portion interacts with Mph-1/Rae28. Therefore, representative Bmi-1 is characterized by interacting with Mph-1/Rae28. Representatively, Bmi-1 has Pro/Ser (a proline/serine-rich sequence at the C terminus thereof (approximately position 248-324 in the sequence of FIG. 1 as set forth in SEQ ID NOs:2 and 4). PcG is linked to neoplastic transformation, lymphocytopoiesis, neurological development, and fibroblast senescence (van der Lugt, N. M. et al., Genes Dev., 8, 757-769 (1994); Lessard, J. et al., Blood, 91, 1216-1224 (1999); Kiyono, T. et al., Nature, 396, 84-88 (1998); and van der Lugt et al., Mech. Dev., 58, 153-164 (1996)). The expression of Bmi-1 is considered to be reduced with the development of hematopoietic cells. Agents in the PcG group are characterized by binding to cis-acting DNA elements, recruiting histone deacetylase, and/or altering chromatin structure. Bmi-1 is known to form a PcG complex.

Cell-type specific gene expression patterns are stabilized by changes in chromatin structure. Cellular memory of chromatin modifications can be faithfully maintained through subsequent cell divisions by the counteractions of transcriptional activators of the trithorax group (TrxG) and proteins and repressors of the polycomb group (PcG) (Jacobs, J. J. L. et al., Biochim. Biophys. Acta 1602, 151-161, 2002; Orland, V. Science 273, 242-245, 2003). PcG proteins form multiprotein complexes that play an important role in the maintenance of transcriptional repression of target genes. At least two distinct PcG complexes have been identified and well characterized. One complex includes Eed, EzH1, and EzH2, and the other includes Bmi-1, Mel-1, Mph1/Rae28, M33, Scmh1, and Ring1A/B. Eed-containing complexes controls gene repression through recruitment of histone deacetylase followed by local chromatin deacetylation, and by methylation of histone H3 Lysine 27 by EzH2. In contrast, no enzymatic activity has yet been reported with regard to Bmi-1-containing complexes. However, Bmi-1-complexes antagonize chromatin remodeling by the SWI-SNF complex (Shao, Z., et al., Cell 98, 37-46, 1999) and are recruited to methylated histone H3 Lysine 27 via M33 chromodomain to contribute to the static maintenance of epigenetic memory (Fischle, W., et al., Genes & Development 17, 1870-1881, 2003). These two types of complexes coordinately maintain positional memory along the anterior-posterior axis by regulating Hox gene expression patterns during development (Jacobs, J. J. L. et al., Biochim. Biophys. Acta 1602, 151-161, 2002; Orland, V. Science 273, 242-245, 2003). On the other hand, these two complexes play reciprocal roles in definitive hematopoiesis: negative regulation by the Eed-containing complex and positive regulation by Bmi-1-containing complex (Lessard, J., et al., Genes & Development 13, 2691-2703, 1999). The Bmi-1-containing complex has been implicated in the maintenance of hematopoietic and leukemic stem cells (Ohta, H., at al., Journal of Experimental Medicine 195, 759-770, 2002; Park, I.-K., et al, Nature 423, 302-305., 2003; Lessard, J. et al., Nature 423, 255-260, 2003). Mph1/Rae28 ¹ fetal liver contains 20-fold fewer long-term lymphohematopoietic repopulating HSCs than wild type (Ohta, H., et al., Journal of Experimental Medicine 195, 759-770, 2002). More importantly, although Bmi-1^{-/-} mice show normal development of embryonic hematopoiesis, Bmi-1^{-/-} HSC have a profound defect in self-renewal capacity. They cannot repopulate the bone marrow (hematopoiesis) in the long-term and it leads to progressive postnatal pancytopenia (Park, I.-K., et al., Nature 423, 302-305., 2003; van derLugt, N. M., et al., Genes & Dev. 8, 757-769, 1994). Notably, the self-renewal defect is not confined to HSC, but also applicable to leukemic stem cells and neuronal stem cells (Lessard, J. at al., Nature 423, 255-260, 2003; Molofsky, A. V., et al, Nature 425, 962-967, 2003). So far, the defective self-renewal of HSC has been attributed to de-repression of Bmi-1 target genes p16^{Ink4a} and p19^{Arf}, and deficiency of these genes partially reverses the self-renewal defect in Bmi-1^{-/-} stem cells (Park, I.-K., et al., Nature 423, 302-305., 2003; van der Lugt, N. M., et al., Genes & Development 8, 757-769, 1994; Molofsky, A. V., et al., Nature 425, 962-967, 2003: Jacobs, J. J. L., et al., Nature 397, 164-168, 1999). More recently, Bmi-1 was reported also to be essential to the expansion of cerebellar granule cell progenitors, in which Bmi-1 expression is reportedly regulated by the sonic hedgehog pathway (Leung, C., et al., Nature 428, 337-341, April 2004). All of these findings have uncovered novel aspects or stem cell regulation exerted by epigenetic modifications. However, the defects in HSC in Bmi-1^{-/-} mice has not yet been characterized in detail at the clonal level in vitro and in vivo. Furthermore, important questions remain, including the role of each component of the PcG complex in HSC and the impact of forced or induced expression of Bmi-1 on HSC self-renewal.

Bmi-1 is known to negatively control expression of p16^{INK4a} and p19^{ARF} (see, for example, Jacobs, J J L, et al., Nature 397, 164-168, 1999). Therefore, it can be determined whether or not a certain agent is Bmi-1 or an equivalent of Bmi-1 in an in vitro experiment, by assaying inhibition of expression of mRNA for p16 or p19 in cells capable of expressing p16 or p19.

It is known that a Bmi-1 homolog is present in drosophila in addition to mammals including human, rat, mouse, and the like. Therefore, as used herein, Bmi-1 usually refers to one that is present in general organisms including mammals and the like.

It is known that a molecule known as “Mel-18”, which is very similar to Bmi-1, is present in mammals. In the present invention, if such a similar molecule is present, itis intended that the similar molecule has a similar function. In a preferred embodiment, the sequence as set forth in SEQ ID NOs:5 and 7 (Accession No. NM_{—}007144 (human) and Accession No. D90085 (mouse), respectively (nucleic acid sequences)) and SEQ ID NOs:6 and 8 (human and mouse, respectively (amino acid sequences)) may be used.

As used herein, “active Bmi-1” refers to Bmi-1 which is in the active state, the molecule being capable of forming a complex with other PcG gene products (e.g., Rae28/Mph1, M33, etc.), binding a cis-acting DNA element, recruiting histone deacetylase, and altering chromatin structure. The active Bmi-1 is characterized by being complexed with, representatively, Rae28/Rph1 or M33, preferably Rae28/Mph1 and M33.

As used herein, “M33” refers to a PcG gene product, which interacts with Bmi-1 and representatively has a sequence as set forth in SEQ ID NO:13 (Accession No. XP_{—}300674, human nucleic acid), 14 (human amino acid), 15 (Accession No. BC035199, mouse nucleic acid), or 16 (mouse amino acid).

As used herein, “Rae28” or “Mph1” are used interchangeably and representatively have a sequence as set forth in SEQ ID NO:9 (Accession No. NM_{—}004426, human nucleic acid), 10 (human amino acid), 11 (Accession No. U63386, mouse nucleic acid), or 12 (mouse amino acid). This gene is also called Rae28/Mph1.

Active Bmi-1 can be artificially or synthetically produced. The artificially produced Bmi-1 includes Bmi-1 having a structure which allows it to be consistently complexed with other PcG gene products. An exemplary active Bmi-1 includes, but is not limited to, a complex with other PcG gene products.

In another embodiment, active Bmi-1 may be a low molecular weight compound (e.g., a product of a combinatorial library). Those skilled in the art can easily screen for such a low molecular weight compound. Screening can be carried out by detecting the ability to complex with other PcG gene products as described herein.

The Bmi-1 of the present invention may be any molecule as long as the molecule has a function of naturally-occurring Bmi-1. Such a function includes, but is not limited to, for example, an ability to complex with Mph-1/Rae28, an ability of nuclear localization, and the like.

As used herein, active Bmi-1 may be any molecule as long as the molecule has a function of a naturally-occurring Bmi-1 complex. It can be determined whether or not an agent is active Bmi-1, by determining whether or not a complex of a candidate agent for Bmi-1 and other PcG gene products is capable of binding to a cis-acting DNA element, whether or not the agent is capable of recruiting histone deacetylase, and/or whether or not the agent is capable of altering chromatin structure. Specifically, when the agent is a nucleic acid, the nuclear translocation of the agent can be determined by transfecting cells with the Bmi-1 gene, immunostaining the Bmi-1 gene using anti-Bmi-1 antibodies, and confirming the nuclear localization of the agent. Alternatively, antibodies for the other PcG gene products can be used to immunologically stain cells transfected with candidate agents and confirm the localization of an agent in nuclei to determine the nuclear translocation. When the agent is a protein or the like, the nuclear translocation of the agent can be determined by introducing the agent directly into cells, and thereafter, immunostaining the agent using antibodies specific to the agent. The above-described techniques are well known in the art.

To determine an ability to bind to Mph-1/Rae28, the binding in a cell is investigated as follows. A cell capable of expressing Mph-1/Rae28 is transfected with a certain agent. A nucleus extract is purified. Antibodies to Mph-1/Rae28 or Bmi-1 are used to perform immunoprecipitation. The binding ability can be determined by detecting when there is a coprecipitate with Bmi-1 or Mph-1/Rae28. Typically, if the formation of a complex is significantly confirmed, the agent can be determined to have at least one function which is the same as that of naturally-occurring Bmi-1.

It is sufficiently determined whether or not Bmi-1 is active, by detecting at least one significant activity thereof.

It is believed that Bmi-1 plays an important role in the expression of the Hox gene and in the formation of axial patterning during the fetal development period. Expression of Bmi-1 has been found in hematopoietic stem cells. Thus, Bmi-1 is expected to play a certain role in hematopoietic stem cells. However, no effect has been known when Bmi-1 is externally inserted into hematopoietic stem cells. The present invention demonstrated that when Bmi-1 is activated, various signals, such as signals inhibiting proliferation, differentiation, cell death, or the like, are transferred to immune cells (T-cells, B-cells, etc.), hematopoietic cells, liver cells, and neural cells. Therefore, it is contemplated in the present invention that the same effect can be obtained by providing conditions which produce active Bmi-1.

As used herein, the term “consistent” in relation to active Bmi-1 means that the function of active Bmi-1 is maintained when no stimulus is applied thereto. As used herein, the term “transiently” in relation to active Bmi-1 means that the function of active Bmi-1 is exhibited only for a certain period. Examples of a consistently active Bmi-1 include, but are not limited to, Bmi-1 modified to be a stable complex, a variant Bmi-1 having a sequence similar to that of a species homolog (e.g., a human homolog, etc.), and the like.

As used herein, the term “ligand” refers to a substance capable of specifically binding to a certain protein. Examples of a ligand include lectin, antigens, antibodies, hormones, neurotransmitters, and the like, which are capable of specifically binding to various receptor protein molecules on cell membranes.

An active Bmi-1 for use in the present invention, includes for example, a portion thereof, to which a sugar chain is attached, including an N-glucoside bond portion to which N-acethyl-D-glucosamine can bind, and an O-glucoside bond portion to which N-acethyl-D-glucosamine can bind (frequently serine or threonine residues). Bmi-1 or active Bmi-1 used herein is not particularly affected by the presence or absence of a sugar chain, though the protein with a sugar chain is typically stable in organisms against decomposition and may have more potent physiological activity. Therefore, the polypeptide with a sugar chain is also encompassed within the present invention.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. The term may include those assembled into a complex of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example. The gene product of the present invention is typically in the form of a polypeptide. The gene product of the present invention in the form of a polypeptide is useful as a composition for the diagnosis, prophylaxis, therapy or prognosis of a disease. In other words, the present invention is useful as a composition in diagnosis embodiments, prophylaxis embodiments, therapy embodiments or prognosis embodiments of the present invention.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uradil, an oligonucleotide derivative in which uracil in an oligonucleotide is substituted with C-s thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Research 19:5081(1991); Ohtsuka et al., Journal of Biological Chemistry 260:2605-2608 (1985); Rossolini et al., Molecular Cellular Probes 8:91-98(1994)). The gene of the present invention is typically in the form of a polynucleotide. The gene or gene product of the present invention in the form of a polynucleotide is useful as a composition for the diagnosis, prophylaxis, therapy or prognosis of the present invention.

As used herein, the term “nucleic acid molecule” is also used interchangeably with the terms “nucleic acid”, “oligonucleotide”, and “polynucleotide”, including cDNA, mRNA, genomic DNA, and the like. As used herein, nucleic acid and nucleic acid molecule may be included by the concept of the term “gene”. A nucleic acid molecule encoding the sequence of a given gene includes “splice mutant (variant)”. Similarly, a particular protein encoded by a nucleic acid encompasses any protein encoded by a splice variant of that nucleic acid. “splice mutants”, as the name suggests, are products of alternative splicing of a gene. After transcription, an initial nucleic acid transcript may be spliced such that different (alternative) nucleic acid splice products encode different polypeptides. Mechanisms for the production of splice variants vary, but include alternative splicing of exons. Alternative polypeptides derived from the same nucleic acid by read-through transcription are also encompassed by this definition. Any products of a splicing reaction, including recombinant forms of the splice products, are included in this definition. Therefore, the gene of the present invention may include the splice mutants herein.

As used herein, “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. Therefore, the term “gene of Bmi-1” or the like typically refers to the structural gene and its transcription and/or translation regulating sequences (e.g., a promoter). In the present invention, it will be understood that regulatory sequences for transcription and/or translation as well as structural genes are useful for diagnosis, therapy, prophylaxis, and prognosis of nerve regeneration, nervous diseases, and the like. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art understand what a gene or a gene product is, according to the context.

As used herein, “homology” of a gene (e g., a nucleic acid sequence, an amino acid sequence, or the like) refers to the proportion of identity between two or more gene sequences. As used herein, the identity of a sequence (a nucleic acid sequence, an amino acid sequence, or the like) refers to the proportion of the identical sequence (an individual nucleic acid, amino acid, or the like) between two or more comparable sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology it the DNA sequences of the genes have representatively at least 50% identity, preferably at least 70% identity, more preferably at least B0%, 90%, 95%, 96%, 97%, 98%, or 99% identity with each other. As used herein, “similarity” of a gene (e.g., a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value.

The similarity, identity and homology of amino acid sequences and base sequences are herein compared using FASTA (sequence analyzing tool) with the default parameters.

As used herein, the term “amino acid” may refer to a naturally-occurring or nonnaturally-occurring amino acid as long as it satisfies the purpose of the present invention. The term “amino acid derivative”or “amino acid analog” refers to an amino acid which is different from a naturally-occurring amino acid and has a function similar to that of the original amino acid. Such amino acid derivatives and amino acid analogs are well known in the art.

The term “naturally-occurring amino acid” refers to an L-isomer of a naturally-occurring amino acid. The naturally-occurring amino-acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise indicated, all amino acids as used herein are L-isomers, although embodiments using. D-amino acids are within the scope of the present invention.

The term “nonnaturally-occurring amino acid” refers to an amino acid which is ordinarily not found in nature. Examples of nonnaturally-occurring amino acids include norleucine, para-nitrophenylalanine, homophenylalanine, para-fluorophenylalanine, 3-amino-2-benzil propionic acid, D- or L-homoarginine, and D-phenylalanine.

The term “amino acid analog” refers to a molecule having a physical property and/or function similar to that of amino acids, but is not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

As used herein, the term “nucleotide” may be either naturally-occurring or nonnaturally-occurring. The term “nucleotide derivative” or “nucleotide analog” refers to a nucleotide which is different from naturally-occurring nucleotides and has a function similar to that of the original nucleotide. Such nucleotide derivatives and nucleotide analogs are well known in the art. Examples of such nucleotide derivatives and nucleotide analogs include, but are not limited to, phosphorothioate, phosphoramidate, methylphosphonate, chiral-methylphosphonate, 2-O-methyl ribonucleotide, and peptide-nucleic acid (PNA).

Amino acids may be referred to herein by either their commonly known three letter symbols or by the one-letter symbols recommended by the IUPAC-IUB Biochemical Nomenclature. Commission. Nucleotides, likewise, may be referred to by their commonly accepted single-letter codes.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to catalytic activity. For example, in the case of antisense molecules, the term refers to a similar portion in an ortholog corresponding to a particular portion of the antisense molecule. Therefore, in the present specification, a particular amino acid sequence of mouse Bmi-1 can be associated with a particular amino acid sequence of human Bmi-1 by analysis (e.g., alignment, etc.). Such a “corresponding” amino acid or nucleic acid may extend over a region or domain having a certain range. Therefore, in this case, such a region or domain is herein referred to as a “corresponding” region or domain.

As used herein, the term “corresponding” gene (e.g., a polypeptide or polynucleotide molecule) refers to a gene (e.g., a polypeptide or polynucleotide molecule) in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, genes corresponding to a mouse Bmi-1 gene and the like can be found in other animals (human, rat, pig, cattle, and the like). Such a corresponding gene can be identified by techniques well known in the art. Therefore, for example, a corresponding gene in a given animal can be found by searching a sequence database containing sequences of the animal (e.g., human, rat) using the sequence of a reference gene (e.g., mouse Bmi-1 genes, and the like) as a query sequence.

As used herein, the term “fragment” refers to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., 10%), as long as the same function is maintained. For this purpose, “about” may be herein put ahead of the numbers. However, it should be understood that the interpretation of numbers is not affected by the presence or absence of “about” in the present specification. The length of a useful fragment may be determined depending on whether or not at least one function (e.g., specific interaction with other molecules, etc.) is maintained among the functions of a full-length protein which is a reference of the fragment.

As used herein, the term “specifically interact with” indicates that a first substance or agent interacts with a second substance or agent with higher affinity than that to substances or agents other than the second substance or agent (particularly, other substances or agents in a sample containing the second substance or agent). Examples of a specific interaction with reference to a substance or agent include, but are not limited to, hybridization of nucleic acids, antigen-antibody reaction, ligand-receptor reaction, enzyme-substrate reaction, a reaction between a transcriptional agent and a binding site of the transcriptional agent when both a nucleic acid and a protein are involved, a protein-lipid interaction, a nucleic acid-lipid interaction, and the like. Therefore, when both the first and second substances or agents are nucleic acids, “specifically interact with” means that the first substance or agent is at least partially complementary to the second substance or agent. Alternatively, when both the first and second substances or agents are proteins, “specifically interact with” includes, but is not limited to, an interaction due to antigen-antibody reaction, an interaction due to receptor-ligand reaction, an enzyme-substrate interaction, and the like. When the two substances or agents are a protein and a nucleic acid, “specifically interact with” includes an interaction between a transcriptional agent and a binding region of a nucleic acid molecule targeted by the transcriptional agent. As used herein, the term “agent capable of specifically interacting with” a biological agent, such as a polynucleotide, a polypeptide or the like, refers to an agent which has an affinity to the biological agent, such as a polynucleotide, a polypeptide or the like, which is representatively higher than or equal to an affinity to other non-related biological agents, such as polynucleotides, polypeptides or the like (particularly, those with identity of less than 30%), and preferably significantly (e.g., statistically significantly) higher. Such an affinity can be measured with, for example, a hybridization assay, a binding assay, or the like. As used herein, the “agent” may be any substance or other agent (e.g., energy, such as light, radiation, heat, electricity, or the like) as long as the intended purpose can be achieved. Examples of such a substance include, but are not limited to, proteins, polypeptides, oligopeptides, peptides, polynucleotides, oligonucleotides, nucleotides, nucleic acids (e.g., DNA such as cDNA, genomic DNA, or the like, and RNA such as mRNA), polysaccharides, oligosaccharides, lipids, low molecular weight organic molecules (e.g., hormones, ligands, information transfer substances, molecules synthesized by combinatorial chemistry, low molecular weight molecules (e.g., pharmaceutically acceptable low molecular weight ligands and the like), and the like), and combinations of these molecules. Examples of an agent specific to a polynucleotide include, but are not limited to, representatively, a polynucleotide having complementarity to the sequence of the polynucleotide with a predetermined sequence homology (e.g., 70% or more sequence identity), a polypeptide such as a transcriptional agent binding to a promoter region, and the like. Examples of an agent specific to a polypeptide include, but are not limited to, representatively, an antibody specifically directed to the polypeptide or derivatives or analogs thereof (e.g., single chain antibody), a specific ligand or receptor when the polypeptide is a receptor or ligand, a substrate when the polypeptide is an enzyme, and the like.

As used herein, the term “compound” refers to any identifiable chemical substance or molecule, including, but not limited to, a low molecular weight molecule, a peptide, a protein, a sugar, a nucleotide, or a nucleic acid. Such a compound may be a naturally-occurring product or a synthetic product.

As used herein, the term “low molecular weight organic molecule” refers to an organic molecule having a relatively small molecular weight. Usually, the low molecular weight organic molecule refers to a molecular weight of about 1,000 or less, or may refer to a molecular weight of more than 1,000. Low molecular weight organic molecules can be ordinarily synthesized by methods known in the art or combinations thereof. These low molecular weight organic molecules may be produced by organisms. Examples of the low molecular weight organic molecule include, but are not limited to, hormones, ligands, information transfer substances, synthesized by combinatorial chemistry, pharmaceutically acceptable low molecular weight molecules (e.g., low molecular weight ligands and the like), and the like.

As used herein, the term “contact” refers to direct or indirect placement of a compound physically close to the polypeptide or polynucleotide of the present invention. Polypeptides or polynucleotides may be present in a number of buffers, salts, solutions, and the like. The term “contact” includes placement of a compound in a beaker, a microtiter plate, a cell culture flask, a microarray (e.g., a gene chip) or the like containing a polypeptide encoded by a nucleic acid or a fragment thereof.

As used herein, the term “antibody” encompasses polyclonal antibodies, monoclonal antibodies, human antibodies, humanized antibodies, polyfunctional antibodies, chimeric antibodies, and anti-idiotype antibodies, and fragments thereof (e.g., F(ab′)2 and Fab fragments), and other recombinant conjugates. These antibodies may be fused with an enzyme (e.g., alkaline phosphatase, horseradish peroxidase, α-galactosidase, and the like) via a covalent bond or by recombination.

As used herein, the term “monoclonal antibody” refers to an antibody composition having a group of homologous antibodies. This term is not limited by the production manner thereof. This term encompasses all immunoglobulin molecules and Fab molecules, F(ab′)2 fragments, Fv fragments, and other molecules having an immunological binding property of the original monoclonal antibody molecule. Methods for producing polyclonal antibodies and monoclonal antibodies are well known in the art, and will be more sufficiently described below.

Monoclonal antibodies are prepared by using standard techniques well known in the art (e.g., Kohler and Milstein, Nature (1975) 256:495) or a modification thereof (e.g., Buck et al. (1982) In vitro 18:377). Representatively, a mouse or rat is immunized with a protein bound to a protein carrier, and boosted. Subsequently, the spleen (and optionally several large lymph nodes) is removed and dissociated into single cells. If desired, the spleen cells may be screened (after removal of nonspecifically adherent cells) by applying the cell suspension to a plate or well coated with a protein antigen. B-cells that express membrane-bound immunoglobulin specific for the antigen bind to the plate, and are not rinsed away with the rest of the suspension. Resulting B-cells, or all dissociated spleen cells, are then induced to fuse with myeloma cells to form hybridomas. The hybridomas are used to produce monoclonal antibodies.

As used herein, the term “antigen” refers to any substrate to which an antibody molecule may specifically bind. As used herein, the term “immunogen” refers to an antigen capable of initiating activation of the antigen-specific immune response of a lymphocyte.

As used herein, the term “single chain antibody” refers to a single chain polypeptide formed by linking the heavy chain fragment and the light chain fragment of the Fv region via a peptide crosslinker.

As used herein, the term “composite molecule” refers to a molecule in which a plurality of molecules, such as polypeptides, polynucleotides, lipids, sugars, low molecular weight molecules, and the like, are linked together. Examples of such a composite molecule include, but are not limited to, glycolipids, glycopeptides, and the like.

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intended nucleic acid; and in the case of proteins, agents other than proteins and proteins having an amino acid sequence other than an intended protein). The “isolated” nucleic acids and proteins include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to a biological agent from which at least a portion of naturally accompanying agents have been removed. Therefore, ordinarily, the purity of a purified biological agent is higher than that of the biological agent in a normal state (i.e., concentrated).

As used herein, the terms “purified” and “isolated” mean that the same type of biological agent is present preferably at least 75% by weight, more preferably at least 85% by weight, even more preferably at least 95% by weight, and most preferably at least 98% by weight.

As used herein, the term “expression” of a gene product, such as a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications.

As used herein, the term “reduction of expression” of a gene, a polynucleotide, a polypeptide, or the like indicates that the level of expression is significantly reduced in the presence of the action of the agent of the present invention, as compared to when the action of the agent is absent. Preferably, the reduction of expression includes a reduction in the amount of expression of a polypeptide (e.g., Bmi-1, or variants or fragments thereof, and the like). As used herein, the term “increase of expression” of a gene, a polynucleotide, a polypeptide, or the like indicates that the level of expression is significantly increased in the presence of the action of the agent of the present invention, as compared to when the action of the agent is absent. Preferably, the increase of expression includes an increase in the amount of expression of a polypeptide (e.g., Bmi-1, or variants or fragments thereof, and the like). As used herein, the term “induction of expression” of a gene indicates that the amount of expression of a gene is increased by applying a given agent to a given cell. Therefore, the induction of expression includes allowing a gene to be expressed when expression of the gene is not otherwise observed, and increasing the amount of expression of the gene when expression of the gene is observed. The increase or reduction of these genes or gene products (polypeptides or polynucleotides) may be useful in treatment embodiments, prognosis embodiments or prophylaxis embodiments of the present invention.

As used herein, the term “specifically expressed” in the case of genes indicates that a gene is expressed in a specific site or for a specific period of time at a level different from (preferably higher than) that in other sites or periods of time. The term “specifically expressed” indicates that a gene may be expressed only in a given site (specific site) or may be expressed in other sites. Preferably, the term “specifically expressed” indicates that a gene is expressed only in a given site. Therefore, according to an embodiment of the present invention, Bmi-1, or variants or fragments thereof, and the like may be expressed specifically or locally in an affected portion (e g., nerve).

As used herein, the term “biological activity” refers to activity possessed by an agent (e.g., a polynucleotide, a protein, etc.) within an organism, including activities exhibiting various functions (e.g., transcription promoting activity). For example, when two agents interact with each other (e.g., Bmi-1 or the like), the biological activity includes binding of the two molecules and a biological change due to the binding. For example, when one molecule is precipitated using antibodies, another molecule may also precipitate. In this case, it is determined that the two molecules are bound together. Specifically, complexation with MphI/Rae-28 is confirmed by co-immunoprecipitation, for example. For example, when a given agent is an enzyme, the biological activity thereof includes the enzymatic activity thereof. In another example, when a given agent is a ligand, the biological activity thereof includes binding of the agent to a receptor for the ligand. Such biological activity can be measured with a technique well known in the art.

As used herein, the term “activity” refers to various measurable indicators which indicate or clarify binding (either directly or indirectly); or affect a response (i.e., having a measurable influence on response to some exposure or stimuli), including the affinity of a compound directly binding to the polypeptide or polynucleotide of the present invention, the amount of an upstream or downstream protein after some stimuli or event, or other similar functional indicator. Such an activity may be measured by an assay, such as competitive inhibition of binding of a Bmi-1-specific agent to Bmi-1.

As used herein, the term “interaction” with reference to two substances means that one substance influences the other substance via forces (e.g., intermolecular forces (Van der Waals force), hydrogen bonding, hydrophobic interactions, or the like). Typically, the two substances interacting with each other interact in the manner of association or binding.

As used herein, the term “binding” means the physical or chemical interaction between two proteins or compounds or associated proteins or compounds or combinations thereof. Binding includes ionic, non-ionic, hydrogen, Van der Waals, hydrophobic interactions, etc. A physical interaction (binding) can be either direct or indirect. Indirect interactions may be through or due to the effects of another protein or compound. Direct binding refers to interactions that do not take place through, or due to, the effect of another protein or compound, but instead are without other substantial chemical intermediates.

As used herein, the term “modulate” or “modify” refers to an increase or decrease or maintenance in a specific activity, or the amount, quality or effect of a protein.

As used herein, “polynucleotides hybridizing under stringent conditions” refers to conditions commonly used and well known in the art. Such a polynucleotide can be obtained by conducting colony hybridization, plaque hybridization, southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution is composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A (adenine) or T (thymine). “Hybridizable polynucleotide” refers to a polynucleotide which can hybridize other polynucleotides under the above-described hybridization conditions. Specifically, the hybridizable polynucleotide includes at least a polynucleotide having a homology of at least 60% to the base sequence of DNA encoding a polypeptide having an amino acid sequence specifically herein disclosed, preferably a polynucleotide having a homology of at least 80%, and more preferably a polynucleotide having a homology of at least 95%.

The term “highly stringent conditions” refers to those conditions that are designed to permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.0015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual (2nd ed., Cold Spring Harbor Laboratory, N.Y., 1989); Anderson et al., Nucleic Acid Hybridization: A Practical Approach Ch. 4 (IRL Press Limited) (Oxford Express). More stringent conditions (such as higher temperature, lower ionic strength, higher formamide, or other denaturing agents) may be optionally used. Other agents may be included in the hybridization and washing buffers for the purpose of reducing non-specific and/or background hybridization. Examples are 0.1% bovine serum albumin 0.1% polyvinylpyrrolidone, 0.1% sodium pyrophosphate, 0.1% sodium dodecylsulfate (NaDodSO₄ or SDS), Ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are ordinarily carried out at pH 6.8-7.4: however, at typical ionic strength conditions, the rate of hybridization is nearly independent of pH. See Anderson et al., Nucleic Acid Hybridization: A Practical Approach Ch. 4 (IRL Press Limited, Oxford UK).

Agents affecting the stability of DNA duplex include base composition, length, and degree of base pair mismatch. Hybridization conditions can be adjusted by those skilled in the art in order to accommodate these variables and allow DNAs of different sequence relatedness to form hybrids. The melting temperature of a perfectly matched DNA duplex can be estimated by the following equation:
Tm(° C.)=81.5+16.6(log[Na⁺])+0.41 (% G+C)−600/N−0.72(% formamide)
where N is the length of the duplex formed, [Na⁺] is the molar concentration of the sodium ion in the hybridization or washing solution, % G+C— is the percentage of (guanine+cytosine) bases in the hybrid. For imperfectly matched hybrids, the melting temperature is reduced by approximately 1° C. for each 1% mismatch.

The term “moderately stringent conditions” refers to conditions under which a DNA duplex with a greater degree of base pair mismatching than could occur under “highly stringent conditions” is able to form. Examples of typical “moderately stringent conditions” are 0.015 M sodium chloride, 0.0015M-sodium-citrate at 50-65° C. or 0.015 M sodium chloride, 0.0015 M sodium citrate, and 20% formamide at 37-50° C. By way of example, “moderately stringent conditions” of 50° C. in 0.015 M sodium ion will allow about a 21% mismatch.

It will be appreciated by those skilled in the art that there is no absolute distinction between “highly stringent conditions” and “moderately stringent conditions”. For example, at 0.015M sodium ion (no formamide), the melting temperature of perfectly matched long DNA is about 71° C. With a wash at 65° C. (at the same ionic strength), this would allow for approximately a 6% mismatch. To capture more distantly related sequences, those skilled in the art can simply lower the temperature or raise the ionic strength.

A good estimate of the melting temperature in 1 M NaCl for oligonucleotide probes up to about 20 nucleotides is given by:
Tm=(2° C. per A-T base pair)+(4° C. per G-C base pair).

Note that the sodium ion concentration in 6×salt sodium citrate (SSC) is 1 M. See Suggs et al., Developmental Biology Using Purified Genes 683 (Brown and Fox, eds., 1981).

A naturally-occurring nucleic acid encoding a protein (e.g., Bmi-1, or variants or fragments thereof, or the like) may be readily isolated from a cDNA library having PCR primers and hybridization probes containing part of a nucleic acid sequence indicated by, for example, SEQ ID NO. 1 (Accession No. L13689) or the like. A preferable nucleic acid encoding Bmi-1, or variants or fragments thereof, or the like is hybridizable to the whole or part of a sequence as set forth in SEQ ID NO:1 or 3 under low stringency conditions defined by hybridization buffer essentially containing 1% bovine serum albumin (BSA); 500 mM sodium phosphate (NaPO₄); 1 mM EDTA; and 7% SDS at 42° C., and wash buffer essentially containing 2×SSC (600 mM NaCl; 60 mM sodium citrate); and 0.1% SDS at 50° C., more preferably under low stringency conditions defined by hybridization buffer essentially containing 1% bovine serum albumin (BSA); 500 mM sodium phosphate (NaPO₄); 15% formamide; 1 mM EDTA; and 7% SDS at 50° C., and wash buffer essentially containing 1×SSC (300 mM NaCl; 30 mM sodium citrate); and 1% SDS at 50° C., and most preferably under low stringency conditions defined by hybridization buffer essentially containing 1% bovine serum albumin (BSA); 200 mM sodium phosphate (NaPO₄); 15% formamide; 1 mM EDTA; and 7% SDS at 50° C., and wash buffer essentially containing 0.5×SSC (150 mM NaCl; 15 mM sodium citrate); and 0.1% SDS at 65° C.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 0.14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al., Journal of Molecular Biology 215:403-410 (1990)), PASTA (Pearson & Lipman, Proceedings of the National Academic Sciences USA 85:2444-2448 (1988)), Smith and Waterman method (Smith and Waterman, Journal of Molecular Biology. 147:195-197 (1981)), and Needleman and Wunsch method (Needleman and Wunsch, J. Mol. Biol. 48:443-453 (1970)), and the like. Examples of a biological search include, but are not limited to, a macroarray in which genomic DNA is attached to a nylon membrane or the like or a microarray (microassay) in which genomic DNA is attached to a glass plate under stringent hybridization, PCR and in situ hybridization conditions, and the like. It is herein intended that Bmi-1 and the like used in the present invention include corresponding genes identified by such an electronic or biological search.

As used herein, the “percentage of sequence identity, homology or similarity (amino acid, nucleotide, or the like)” is determined by comparing two optimally aligned sequences over a window of comparison, wherein the portion of a polynucleotide or polypeptide sequence in the comparison window may comprise additions or deletions (i.e. gaps), as compared to the reference sequences (which does not comprise additions or deletions (if the other sequence includes an addition, a gap may occur)) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e. the window size) and multiplying the results by 100 to yield the percentage of sequence identity. When used in a search, homology is evaluated by an appropriate technique selected from various sequence comparison algorithms and programs well known in the art. Examples of such algorithms and programs include, but are not limited to, TBLASTN, BLASTP, FASTA, TFASTA and CLUSTALW (Pearson and Lipman, 1988, Proc. Natl. Acad. Sci. USA 85(8):2444-2448, Altschul et al., 1990, J. Mol. Biol. 215(3):403-410, Thompson et al., 1994, Nucleic Acids Res. 22(2):4673-4680, Higgins et al., 1996, Methods Enzymol. 266:383-402, Altschul et al., 1990, J. Mol. Biol. 215(3):403-410, Altschul et al., 1993, Nature Genetics 3:266-272). In a particularly preferable embodiment, the homology of a protein or nucleic acid sequence is evaluated using a Basic Local Alignment Search Tool (BLAST) well known in the art (e.g., see Karlin and Altschul, 1990, Proc. Natl. Acad. Sci. USA 87:2267-2268, Altschul et al., 1990, J. Mol. Biol. 215:403-410, Altschul et al., 1993, Nature Genetics 3:266-272, Altschul et al., 1997, Nuc. Acids Res. 25:3389-3402). Particularly, 5 specialized-BLAST programs may be used to perform the following tasks to achieve comparison or search;
(1) comparison of an amino acid query sequence with a protein sequence database using BLASTP and BLAST3;
(2) comparison of a nucleotide query sequence with a nucleotide sequence database using BLASTN;
(3) comparison of a conceptually translated product in which a nucleotide query sequence (both strands) is converted over 6 reading frames with a protein sequence database using BLASTX;
(4) comparison of all protein query sequences converted over 6 reading frames (both strands) with a nucleotide sequence database using TBLASTN; and
(5) comparison of nucleotide query sequences converted over 6 reading frames with a nucleotide sequence database using TBLASTX.

The BLAST program identifies homologous sequences by specifying analogous segments called “high score segment pairs” between amino acid query sequences or nucleic acid query sequences and test sequences obtained from preferably a protein sequence database or a nucleic acid sequence database. A large number of the high score segment pairs are preferably identified (aligned) using a scoring matrix well known in the art. Preferably, the scoring matrix is the BLOSUM62 matrix (Gonnet et al., 1992, Science 256:1443-1445, Henikoff and Henikoff, 1993, Proteins 17:49-61). The PAM or PAM250 matrix may be used, although they are not as preferable as the BLOSUM62 matrix (e.g., see Schwartz and Dayhoff, eds., 1978, Matrices for Detecting Distance Relationships: Atlas of Protein Sequence and Structure, Washington: National Biomedical Research Foundation). The BLAST program evaluates the statistical significance of all identified high score segment pairs and preferably selects segments which satisfy a threshold level of significance independently defined by a user, such as a user set homology. Preferably, the statistical significance of high score segment pairs is evaluated using Karlin's formula (see Karlin and Altschul, 1990, Proc. Natl. Acad. Sci. USA 87:2267-2268).

As used herein, the term “primer” refers to a substance required for initiation of a reaction of a macromolecule compound to be synthesized, in a macromolecule synthesis enzymatic reaction. In a reaction for synthesizing a nucleic acid molecule, a nucleic acid molecule (e.g., DNA, RNA, or the like) which is complementary to part of a macromolecule compound to be synthesized may be used.

A nucleic acid molecule which is ordinarily used as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 1-8 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, and most preferably at least 95%. An appropriate sequence as a primer may vary depending on the property of the sequence to be synthesized (amplified). Those skilled in the art can design an appropriate primer depending on the sequence of interest. Such a primer design is well known in the art and may be performed manually or using a computer program (e.g., LASERGENE, Primer Select, DNAStar).

As used herein, the term “epitope” refers to an antigenic determinant whose structure is clear. Therefore, the term “epitope” includes a set of amino acid residues which are involved in recognition by a particular immunoglobulin, or in the context of T cells, those residues necessary for recognition by T cell receptor proteins and/or Major Histocompatibility Complex (MHC) receptors. This term is also used interchangeably with “antigenic determinant” or “antigenic determinant site”. In the field of immunology, in vivo or in vitro, an epitope is the feature of a molecule (e.g., primary, secondary and tertiary peptide structure, and charge) that forms a site recognized by an immunoglobulin, T cell receptor or MHC (e.g. HLA) molecule. An epitope including a peptide comprises 3 or more amino acids in a spatial conformation which is unique to the epitope. Generally, an epitope consists of at least 5 such amino acids, and more ordinarily, consists of at least 6, 7, 8, 9 or 10 such amino acids. The greater the length of an epitope, the more the similarity of the epitope to the original peptide, i.e., longer epitopes are generally preferable. This is not necessarily the case when the conformation is taken into account. Methods of determining the spatial conformation of amino acids are known in the art, and include, for example, X-ray crystallography and two-dimensional nuclear magnetic resonance spectroscopy. Furthermore, the identification of epitopes in a given protein is readily accomplished using techniques well known in the art. See, also, Geysen et al., Proc. Natl. Acad. Sci. USA (1984) 81: 3998 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23: 709 (technique for identifying peptides with high affinity for a given antibody). Antibodies that recognize the same epitope can be identified in a simple immunoassay. Thus, methods for determining an epitope including a peptide are well known in the art. Such an epitope can be determined using a well-known, common technique by those skilled in the art if the primary nucleic acid or amino acid sequence of the epitope is provided.

Therefore, an epitope including a peptide requires a sequence having a length of at least 3 amino acids, preferably at least 4 amino acids, more preferably at least 5 amino acids, at least 6 amino acids, at least 7 amino acids, at least 8 amino acids, at least 9 amino acids, at least 10 amino acids, at least 15 amino acids, at least 20 amino acids, and at least 25 amino acids. Epitopes may be linear or conformational.

(Modification of Genes, Protein Molecules, Nucleic Acid Molecules, and the Like)

In a given protein molecule (e.g., Bmi-1, etc.), a given amino acid contained in a sequence may be substituted with another amino acid in a protein structure, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA code sequence level, to produce a protein which maintains the original property after the substitution. Therefore, various modifications of peptides as disclosed herein and DNA encoding such peptides may be performed without clear losses of biological usefulness.

When the above-described modifications are designed, the hydrophobicity indices of amino acids may be taken into consideration. The hydrophobic amino acid indices play an important role in providing a protein with an interactive biological function, which is generally recognized in the art (Kyte. J and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8) tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within ±2, more preferably within ±1, and even more preferably within ±0.5. It is understood in the art that such an amino acid substitution based on hydrophobicity is efficient.

A hydrophilicity index is also useful for modification of an amino acid sequence of the present invention. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, conservative substitution is carried out between amino acids having a hydrophilicity or hydrophobicity index of within ±2, preferably within ±1, and more preferably within ±0.5. Examples of conservative substitution include, but are not limited to, substitutions within each of the following residue pairs: arginine and lysine; glutamic acid and aspartic acid; serene and threonine; glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Examples of such a variant include, but are not limited to, a nucleotide or polypeptide having one or several substitutions, additions and/or deletions or a nucleotide or polypeptide having at least one substitution, addition and/or deletion. The term “allele” as used herein refers to a genetic variant located at a locus identical to a corresponding gene, where the two genes are distinguished from each other. Therefore, the term “allelic variant” as used herein refers to a variant which has an allelic relationship with a given gene. Such an allelic variant ordinarily has a sequence the same as or highly similar to that of the corresponding allele, and ordinarily has almost the same biological activity, though it rarely has different biological activity. The term “species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining such a species homolog is clearly understood from the description of the present specification. The term “orthologs” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variation. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid capable of largely affecting the higher-order structure of a polypeptide. Examples of a method for such modification of a base sequence include cleavage using a restriction enzyme or the like; ligation or the like by treatment using DNA polymerase, Klenow fragments, DNA ligase, or the like; and a site specific base substitution method using synthesized oligonucleotides (specific-site directed mutagenesis; Mark Zoller and Michael Smith, Methods in Enzymology, 100, 468-500(1983)). Modification can be performed using methods ordinarily used in the field of molecular biology.

In order to prepare functionally equivalent polypeptides, amino acid additions, deletions, or modifications can be performed in addition to amino acid substitutions. Amino acid substitution(s) refers to the replacement of at least one amino acid of an original peptide chain with different amino acids, such as the replacement of 1 to 10 amino acids, preferably 1 to 5 amino acids, and more preferably 1 to 3 amino acids with different amino acids. Amino acid addition(s) refers to the addition of at least one amino acid to an original peptide chain, such as the addition of 1 to 10 amino acids, preferably 1 to 5 amino acids, and more preferably 1 to 3 amino acids to an original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modification includes, but is not limited to, amidation, carboxylation, sulfation, halogenation, truncation, lipidation, alkylation, glycosylation, phosphorylation, hydroxylation, acylation (e.g., acetylation), and the like. Amino acids to be substituted or added may be naturally-occurring or nonnaturally-occurring amino acids, or amino acid analogs, Naturally-occurring amino acids are preferable.

As used herein, the term “peptide analog” or “peptide derivative” refers to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution that the function thereof is substantially the same as the function of the original peptide (e.g., a similar pKa value, a similar functional group, a similar binding manner to other molecules, a similar water-solubility, and the like). Such a peptide analog can be prepared using a technique well known in the art. Therefore, a peptide analog may be a polymer containing an amino acid analog.

A chemically-modified polypeptide composition in which a polypeptide of the present invention is attached to a polymer is included within the scope of the present invention. This polymer may be water soluble so that the protein does not precipitate in an aqueous environment (e.g., a physiological environment). An appropriate water soluble polymer may be selected from the group consisting of: polyethylene glycol (PEG), monomethoxy-polyethylene glycol, dextran, cellulose, or other carbohydrate based polymers, poly-(N-vinylpyrrolidone)polyethylene glycol, propylene glycol homopolymers, a polypropylene oxide/ethylene oxide co-polymer, polyoxyethylated polyols (e.g., glycerol) and polyvinyl alcohol. The selected polymer is typically modified to have a single reactive group (e.g., active ester for acylation or aldehyde for alkylation). As a result, the degree of polymerization may be controlled. The polymer may be of any molecular weight, and may be branched or unbranched. Included within the scope of suitable polymers is a mixture of polymers. When the chemically modified polymer of the present invention is used in therapeutic applications, a pharmaceutically acceptable polymer is selected.

When the polymer is modified by an acylation reaction, the polymer should have a single reactive ester group. Alternatively, when the polymer is modified by reducing alkylation, the polymer should have a single reactive aldehyde group. A preferable reactive aldehyde is, for example, polyethylene glycol, propionaldehyde (which is water stable), or mono C₁-C₁₀ alkoxy or aryloxy derivatives thereof (see U.S. Pat. No. 5,252,714, which is herein incorporated by reference in its entirety).

Pegylation of the polypeptide of the present invention may be carried out by any of the pegylation reactions known in the art, as described for example in the following references: Focus on Growth Factors, 3, 4-10 (1992); EP 0 154 316; EP 0 401 384, which are herein incorporated by reference in their entirety). Preferably, pegylation may be carried out via an acylation reaction or an alkylation reaction with a reactive polyethylene glycol molecule (or an analogous reactive water-soluble polymer). Polyethylene glycol (PEG) is a water-soluble polymer suitable for use in pegylation of the polypeptide of the present invention (e.g., Bmi-1, Mel-18, M33, Mph-1/Rae28, and the like). As used herein, the term “polyethylene glycol” is meant to encompass any of the forms of PEG that have been used to derivatize proteins (e.g., mono(C1-C10) alkoxy-polyethylene glycol or mono(C1-C10) aryloxy-polyethylene glycol (PEG)).

Chemical derivatization of the polypeptide of the present invention may be performed under any suitable conditions that can be used to react a biologically active substance with an activated polymer molecule. Methods for preparing pegylated polypeptides of the present invention will generally comprise the steps of (a) reacting the polypeptide with polyethylene glycol (such as a reactive ester or aldehyde derivative of PEG) under conditions whereby Bmi-1 becomes attached to one or more PEG groups, and (b) obtaining the reaction product (s). The optimal reaction conditions or the acylation reactions are easily selected by those skilled in the art based on known parameters and the desired result.

Generally, conditions may be alleviated or modulated by the administration of the pegylated polypeptide of the present invention. However, the polypeptide derivative of the polypeptide molecule of the present invention disclosed herein may have additional activities, enhanced or reduced biological activity, or other characteristics (e.g., increased or decreased half-life), as compared to the nonderivatized molecules. The polypeptide of the present invention, and fragments, variants and derivatives thereof may be used singly or in combination, or in combination with other pharmaceutical compositions, such as cytokines, proliferating agents, antigens, anti-inflammatory agents and/or chemotherapeutics, which are suitable for treatment of the symptoms.

Similarly, the term “polynucleotide analog” or “nucleic acid analog” refers to a compound which is different from a polynucleotide or a nucleic acid but has at least one chemical function or biological function equivalent to that of a polynucleotide or a nucleic acid. Therefore, a polynucleotide analog or a nucleic acid analog includes one that has at least one nucleotide analog or nucleotide derivative addition or substitution with respect to the nucleic acid sequence encoding the original peptide.

Nucleic acid molecules as used herein includes one in which a part of the sequence of the nucleic acid is deleted or is substituted with other base(s), or an additional nucleic acid sequence is inserted, as long as a polypeptide expressed by the nucleic acid has substantially the same activity as that of the naturally-occurring polypeptide, as described above. Alternatively, an additional nucleic acid may be linked to the 5′ terminus and/or 3′ terminus of the nucleic acid. The nucleic acid molecule may include one that is hybridizable to a gene encoding a polypeptide under stringent conditions and encodes a polypeptide having substantially the same function. Such a gene is known in the art and can be used in the present invention.

The above-described nucleic acid can be obtained by a well-known PCR method, i.e., chemical synthesis. This method may be combined with, for example, site-specific mutagenesis, hybridization, or the like.

As used herein, the term “substitution, addition or deletion” for a polypeptide or a polynucleotide refers to the substitution, addition or deletion of an amino acid or its substitute, or a nucleotide or its substitute, with respect to the original polypeptide or polynucleotide, respectively. This is achieved by techniques well known in the art, including a site-specific mutagenesis technique and the like. A polypeptide or a polynucleotide may have any number (>0) of substitutions, additions, or deletions. The number can be as large as a variant having such a number of substitutions, additions or deletions which maintains an intended function (e.g., the information transfer function of hormones and cytokines, etc.). For example, such a number may be one or several, and preferably within 20% or 10% of the full length, or no more than 100, no more than 50, no more than 25, or the like.

(General Techniques)

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-Interscience; Ausubel, F. M. (1989), Short Protocols in Molecular Biology; A Compendium of Methods from Current Protocols in Molecular Biology, Greene Pub. Associates and Wiley-Interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Experimental Method for Gene Introduction & Expression Analysis”, Yodo-sha, 1997; and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

(Genetic Engineering)

Bmi-land the like, and fragments and variants thereof as used herein can be produced by genetic engineering techniques.

When a gene is mentioned herein, the term “vector” or “recombinant vector” refers to a vector capable of transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable for cloning is referred to as “cloning vector”. Such a cloning vector ordinarily contains a multiple cloning site containing a plurality of restriction sites. Restriction sites and multiple cloning sites are well known in the art and may be appropriately or optionally used depending on the purpose. The technology is described in references as described herein (e.g., Sambrook et al. (supra)). Preferred vectors include, but are not limited to, plasmids, phages, cosmids, episomes, viral particles or viruses, and integratable DNA fragments (i.e., fragments which can be integrated into a host genome by homologous recombination) Preferred viral particles include, but are not limited to, adenoviruses, baculoviruses, parvoviruses, herpesviruses, poxviruses, adeno-associated viruses, Semliki Forest viruses, vaccinia viruses, and retroviruses.

One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers. It is well known to those skilled in the art that the type of organism (e.g., a plant) expression vector and the type of regulatory element may vary depending on the host cell.

As used herein, a “recombinant vector” for prokaryotic cells includes, for example, pcDNA 3(+), pBluescript-SK(+/−), pGEM-T, pEF-BOS, pEGFP, pHAT, pUC18, pFT-DEST™, 42GATEWAY (Invitrogen), and the like.

As used herein, a “recombinant vector” for animal cells includes, for example, pcDNA I/Amp, pcDNA I, pCDMS (all commercially available from Funakoshi, Tokyo, Japan), pAGE107 [Japanese Laid-Open Publication No. 3-229 (Invitrogen)], pAGE103 [J. Biochem., 101, 1307 (1987)], pAMo, pAMoA [J. Biol. Chem., 268, 22782-22787 (1993)], retroviral expression vectors based on Murine Stem Cell Virus (MSCV), pEF-BOS, pEGEP, and the like.

As used herein, the term “terminator” refers to a sequence which is located downstream of a protein-encoding region of a gene and which is involved in the termination of transcription when DNA is transcribed into mRNA, and the addition of a poly A sequence. It is known that a terminator contributes to the stability of mRNA, and has an influence on the amount of gene expression.

As used herein, the term “promoter” refers to a base sequence which determines the initiation site of transcription of a gene and is a DNA region which directly regulates the frequency of transcription. Transcription is started by RNA polymerase binding to a promoter. Therefore, a portion of a given gene which functions as a promoter is herein referred to as a “promoter portion”. A promoter region is usually located within about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but depending on the structural gene, i.e., a putative promoter region may be located downstream of a structural gene. Preferably, a putative promoter region is located within about 2 kbp upstream of the translation initiation site of the first exon.

As used herein, the term “origin of replication” refers to a specific region on a chromosome from which DNA replication starts. An origin of replication may be provided either by construction of the vector so that an endogenous origin is contained therein or by the chromosomal replication mechanism of a host cell. When the vector is integrated into a chromosome in the host cell, the latter may be sufficient. Alternatively, instead of using a vector containing a viral origin of replication, a mammalian cell may be transformed by those skilled in the art using a method of co-transforming a selectable marker and the DNA of the present invention. Examples of an appropriate selectable marker include dihydrofolate reductase (DHFR) or thymidine kinase (U.S. Pat. No. 4,399,216).

For example, by expressing a nucleic acid using a tissue-specific regulatory element, a recombinant mammalian expression vector is capable of directing expression of the nucleic acid preferentially in a particular cell type. Tissue-specific regulatory elements are known in the art. Non-limiting examples of suitable tissue-specific promoters include developmentally-regulated promoters (e.g., the murine hox promoters (Kessel and Gruss (1990) Science 249, 374-379) and the alpha-fetoprotein promoter (Campes and Tilghman (1989) Genes & Development 3, 531-546); the albumin promoter (liver-specific: Pinkert et al. (1987) Genes Dev 1, 268-277), lymphoid-specific promoters (Calame and Eaton (1988) Adv Immunol 43, 235-275), in particular promoters of T cell receptors (Winoto and Baltimore (1989) EMBO J. 8, 729-733) and immunoglobulins (Banerji et al. (1983) Cell 33, 729-740; Queen and Baltimore (1983) Cell 33, 741-748), neuron-specific promoters (e.g., the neurofilament promoter; Byrne and Ruddle (1989) Proc. Natl. Acad. Sci. USA 86, 5473-5477), pancreas-specific promoters (Edlund et al. (1985) Science 230, 912-916), and mammary gland-specific promoters (e.g., milk whey promoter; U.S. Pat. No. 4,873,316 and European Application Publication No. 264, 166).

As used herein, the term “enhancer” refers to a sequence which is used so as to enhance the expression efficiency of a gene of interest. Such an enhancer is well known in the art. One or more enhancers may be used, or no enhancer may be used.

As used herein, the term “operatively linked” indicates that a desired sequence is located such that expression (operation) thereof is under control of a transcription and translation regulatory sequence (e.g., a promoter, an enhancer, and the like) or a translation regulatory sequence. In order for a promoter to be operatively linked to a gene, typically, the promoter is located immediately upstream of the gene. A promoter is not necessarily adjacent to a structural gene.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y., Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] “Experimental Method for Gene Introduction & Expression Analysis”, Yodo-sha, 1997; and the like. Gene introduction can be confirmed by methods as described herein, such as Northern blotting analysis and Western blotting analysis, or other well-known, common techniques.

Any of the above-described methods for introducing DNA into cells can be used as a vector introduction method, including, for example, transfection, transduction, transformation, and the like (e.g., a calcium phosphate method, a liposome method, a DEAE dextran method, an electroporation method, a particle gun (gene gun) method, and the like).

As used herein, the term “transformant” refers to the whole or a part of an organism, such as a cell, which is produced by transformation. Examples of a transformant include a prokaryotic cell, yeast, an animal cell, a plant cell, an insect cell, and the like. Transformants may be referred to as transformed cells, transformed tissue, transformed hosts, or the like, depending on the subject. A cell used herein may be a transformant.

When a prokaryotic call is used herein for genetic operations or the like, the prokaryotic cell may be, for example, of the genus Escherichia, Serratia, Bacillus, Brevibacterium, Corynebacterium, Microbacterium, Pseudomonas, or the like. Specifically, the prokaryotic cell is, for example, Escherichia coli XL1-Blue, Escherichia coli XL2-Blue, Escherichia coli DH1, or the like.

Examples of an animal cell as used herein include a mouse myeloma cell, a rat myeloma cell, a mouse hybridoma cell, a Chinese hamster ovary (CHO) cell, a baby hamster kidney (BHK) cell, an African green monkey kidney cell, a human leukemic cell, HBT5637 (Japanese Laid-Open Publication No. 63-299), a human colon cancer cell line, and the like. The mouse myeloma cell includes ps20, NSO, and the like. The rat myeloma cell includes YB2/0 and the like. A human embryonic kidney cell includes HEK293 (ATCC:CRL-1573) and the like. The human leukemic cell includes BALL-1 and the like. The African green monkey kidney cell includes COS-1, COS-7, and the like. The human colon cancer cell line includes HCT-15, and the like. A human neuroblastoma includes SK-N-SH, SK-N-SH-5Y, and the like. A mouse neuroblastoma includes Neuro2A, and the like.

Any method for introduction of DNA can be used herein as a method for introduction of a recombinant vector, including, for example, a calcium chloride method, an electroporation method (Methods. Enzymol., 194, 182 (1990)), a lipofection method, a spheroplast method (Proc. Natl. Acad. Sci. USA, 84, 1929 (1978)), a lithium acetate method (J. Bacteriol., 153, 163 (1983)), a method described in Proc. Natl. Acad. Sci. USA, 75, 1929 (1978), and the like.

A retrovirus infection method as used herein is well known in the art as described in, for example, Current Protocols in Molecular Biology (supra) (particularly, Units 9.9-9.14), and the like. Specifically, for example, embryonic stem cells are trypsinized into a single-cell suspension, followed by co-culture with the culture supernatant of virus-producing cells (packaging cell lines) for 1-2 hours, thereby obtaining a sufficient amount of infected cells.

The transient expression of Cre enzyme, DNA mapping on a chromosome, and the like, which are used herein in a method for removing a genome, a gene locus, or the like, are well known in the art, as described in Kenichi Matsubara and Hiroshi Yoshikawa, editors, Saibo-Kogaku [Cell Engineering], special issue, “Experiment Protocol Series “FISH Experiment Protocol From Human Genome Analysis to Chromosome/Gene diagnosis”, Shujun-sha (Tokyo), and the like.

Gene expression (e.g., mRNA expression, polypeptide expression) may be “detected” or “quantified” by an appropriate method, including mRNA measurement and immunological measurement methods. Examples of the molecular biological measurement method include a Northern blotting method, a dot blotting method, a PCR method, and the like, Examples of the immunological measurement method include an ELISA method, an RIA method, a fluorescent antibody method, a Western blotting method, an immunohistological staining method, and the like, where a microtiter plate may be used. Examples of a quantification method include an ELISA method, an RIA method, and the like. A gene analysis method using an array (e.g., a DNA array, a protein array, etc.) may be used. The DNA array is widely reviewed in Saibo-Kogaku [Cell Engineering], special issue, “DNA Microarray and Up-to-date PCR Method”, edited by Shujun-sha. The protein array is described in detail in Nature Genetics 2002 December; 32 Suppl:526-32. Examples of a method for analyzing gene expression include, but are not limited to, a RT-PCR method, a RACE method, a SSCP method, an immunoprecipitation method, a two-hybrid system, an in vitro translation method, and the like in addition to the above-described techniques.

Other analysis methods are described in, for example, “Genome Analysis Experimental Method, Yusuke Nakamura's Labo-Manual, edited by Yusuke Nakamura, Yodo-sha (2002), and the like. All of the above-described publications are herein incorporated by reference.

As used herein, the term “amount of expression” refers to the amount of a polypeptide or mRNA expressed in a subject cell. The amount of expression includes the amount of expression at the protein level of a polypeptide of the present invention evaluated by any appropriate method using an antibody of the present invention, including immunological measurement methods (e.g., an ELISA method, a RIA method, a fluorescent antibody method, a Western blotting method, an immunohistological staining method, and the like, or the amount of expression at the mRNA level of a polypeptide of the present invention evaluated by any appropriate method, including molecular biological measurement methods (e.g., a Northern blotting method, a dot blotting method, a PCR method, and the like). The term “change in the amount of expression” indicates that an increase or decrease in the amount of expression at the protein or mRNA level of a polypeptide of the present invention evaluated by an appropriate method including the above-described immunological measurement method or molecular biological measurement method.

As used herein, the term “upstream” in reference to a polynucleotide means that the position is closer to the 5′ terminus than a specific reference point.

As used herein, the term “downstream” in reference to a polynucleotide means that the position is closer to the 3′ terminus than a specific reference point.

As used herein, the term “base paired” and “Watson & Crick base paired” have the same meaning and refer to nucleotides which can be bound together by hydrogen bonds based on the sequence identity that an adenine residue (A) is bound to a thymine residue (T) or a uracil residue (U) via two hydrogen bonds and a cytosine residue (C) is bound to a guanine reside (G) via three hydrogen bonds, as seen in double-stranded DNA (see Stryer, L. Biochemistry, 4th edition, 1995).

As used herein, the term “complementary” or “complement” refers to a polynucleotide sequence such that the whole complementary region thereof is capable of Watson-Crick base paring with another specific polynucleotide. In the present invention, when each base of a first polynucleotide pairs with a corresponding complementary base, the first polynucleotide is regarded as being complementary to a second polynucleotide. Complementary bases are generally A and T (or A and U) or C and G. As used herein, the term “complement” is used as a synonym for the terms “complementary polynucleotide”, “complementary nucleic acid” and “complementary nucleotide sequence”. These terms are applied to a pair of polynucleotides based on the sequence, but not a specific set of two polynucleotides which are virtually bound together.

(Polypeptide Production Method)

A transformant derived from a microorganism, an animal cell, or the like, which possesses a recombinant vector into which DNA encoding a polypeptide of the present invention (e.g., Bmi-1 or a variant or fragment thereof, etc.) is incorporated, is cultured according to an ordinary culture method. The polypeptide of the present invention is produced and accumulated. The polypeptide of the present invention is collected from the culture, thereby making it possible to produce the polypeptide of the present invention.

The transformant of the present invention can be cultured on a culture medium according to an ordinary method for use in culturing host cells. A culture medium for a transformant obtained from a prokaryote (e.g., E. coli) or a eukaryote (e.g., yeast) as a host may be either a naturally-occurring culture medium or a synthetic culture medium as long as the medium contains a carbon source, a nitrogen source, inorganic salts, and the like, which an organism of the present invention can assimilate and the medium allows efficient culture of the transformant.

The carbon source includes any one that can be assimilated by the organism, such as carbohydrates (e.g, glucose, fructose, sucrose, molasses containing the same, starch, starch hydrolysate, and the like), organic acids (e.g., acetic acid, propionic acid, and the like), alcohols (e.g., ethanol, propanol, and the like), and the like.

The nitrogen source includes ammonium salts of inorganic or organic acids (e.g., ammonia, ammonium chloride, ammonium sulfate, ammonium acetate, ammonium phosphate, and the like), and other nitrogen-containing substances (e.g., peptone, meat extract, yeast extract, corn steep liquor, casein hydrolysate, soybean cake, and soybean cake hydrolysate, various fermentation bacteria and digestion products thereof), and the like.

Salts of inorganic acids, such as potassium (I) phosphate, potassium (II) phosphate, magnesium phosphate, sodium chloride, iron (I) sulfate, manganese sulfate, copper sulfate, calcium carbonate, and the like, can be used. Culture is performed under aerobic conditions for shaking culture, deep aeration agitation culture, or the like.

Culture temperature is preferably 15 to 40° C., culture time is ordinarily 5 hours to 7 days. The pH of culture medium is maintained at 3.0 to 9.0. The adjustment of pH is carried out using inorganic or organic acid, alkali solution, urea, calcium carbonate, ammonia, or the like. An antibiotic, such as ampicillin, tetracycline, or the like, may be optionally added to the culture medium during cultivation.

When culturing a microorganism which has been transformed using an expression vector containing an inducible promoter, the culture medium may be optionally supplemented with an inducer. For example, when a microorganism, which has been transformed using an expression vector containing a lac promoter, is cultured, isopropyl-β-D-thiogalactopyranoside or the like may be added to the culture medium. When a microorganism, which has been transformed using an expression vector containing a trp promoter, is cultured, indole acrylic acid or the like may be added to the culture medium. A cell or an organ into which a gene has been introduced can be cultured in a large volume using a jar fermenter.

For example, when an animal cell is used, a culture medium of the present invention for culturing the cell includes a commonly used RPMI1640 culture medium (The Journal of the American Medical Association, 199, 519 (1967)), Eagle's MEM culture medium (Science, 122, 501 (1952)), DMEM culture medium (Virology, 8, 396 (1959)), 199 culture medium (Proceedings of the Society for the Biological Medicine, 73, 1 (1950)) or these culture media supplemented with fetal bovine serum or the like.

Culture is normally carried out for 1 to 7 days in media of pH 6 to 8, at 25 to 40° C., in an atmosphere of 5% CO₂, for example. An antibiotic, such as kanamycin, penicillin, streptomycin, or the like may be optionally added to culture medium during cultivation.

A polypeptide of the present invention can be isolated or purified from a culture of a transformant, which has been transformed with a nucleic acid sequence encoding the polypeptide, using an ordinary method for isolating or purifying enzymes, which are well known and commonly used in the art. For example, when a polypeptide of the present invention is secreted outside a transformant for producing the polypeptide, the culture is subjected to centrifugation or the like to obtain the soluble fraction. A purified specimen can be obtained from the soluble fraction by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Chemical Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.), and the like.

When a polypeptide (e.g., Bmi-1, or a variant or fragment thereof, and the like) of the present invention is accumulated in a dissolved form within a transformant cell for producing the polypeptide, the culture is subjected to centrifugation to collect cells in the culture. The cells are washed, followed by pulverization of the cells using a ultrasonic pulverizer, a French press, MANTON GAULIN homogenizer, Dinomil, or the like, to obtain a cell-free extract solution. A purified specimen can be obtained from a supernatant obtained by centrifuging the cell-free extract solution or by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Chemical Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.) and the like.

When the polypeptide of the present invention has been expressed and formed insoluble bodies within cells, the cells are harvested, pulverized, and centrifuged. From the resulting precipitate fraction, the polypeptide of the present invention is collected using a commonly used method. The insoluble polypeptide is solubilized using a polypeptide denaturant. The resulting solubilized solution is diluted or dialyzed into a denaturant-free solution or a dilute solution, where the concentration of the polypeptide denaturant is too low to denature, the polypeptide. The polypeptide of the present invention is allowed to form a normal three-dimensional structure, and the purified specimen is obtained by isolation and purification as described above.

Purification can be carried out in accordance with a commonly used protein purification method (J. Evan. Sadler et al.: Methods in Enzymology, 83, 458). Alternatively, the polypeptide of the present invention can be fused with other proteins to produce a fusion protein, and the fusion protein can be purified using affinity chromatography using a substance having affinity to the fusion protein (Akio Yamakawa, Experimental Medicine, 13, 469-474 (1995)). For example, in accordance with a method described in Lowe et al., Proc. Natl. Acad. Sci., USA, 86, 8227-8231 (1989), Genes Develop., 4, 1288(1990)), a fusion protein of the polypeptide of the present invention with protein A is produced, followed by purification with affinity chromatography using immunoglobulin G.

A fusion protein of the polypeptide of the present invention with a FLAG peptide is produced, followed by purification with affinity chromatography using anti-FLAG antibodies (Proc. Natl. Acad. Sci., USA, 86, 8227(1989), Genes Develop., 4, 1288 (1990)). For such a fusion protein, in fusion expression vectors, a proteolytic cleavage site is introduced at the junction of a fusion moiety and a recombinant protein to enable separation of the recombinant protein from the fusion moiety subsequent to purification of the fusion protein. Such enzymes, and their cognate recognition sequences, include Factor Xa, thrombin, and enterokinase. Typical fusion expression vectors include pGEX (Pharmacia Biotech Inc; Smith and Johnson (1988) Gene 67, 31-40), pMAL (New England Biolabs. Beverly, Mass.) and pRIT5 (Pharmacia, Piscataway. N. J.) that fuse glutathione S-transferase (GST), maltose E binding protein, or protein A, respectively, to the target recombinant protein.

The polypeptide of the present invention can be purified with affinity chromatography using antibodies which bind to the polypeptide. The polypeptide of the present invention can be produced using an in vitro transcription/translation system in accordance with a known method (J. Biomolecular NMR, 6, 129-134; Science, 242, 1162-1164; J. Biochem., 110, 166-168 (1991)).

The polypeptide of the present invention can also be produced by a chemical synthesis method, such as the Fmoc method (fluorenylmethyloxycarbonyl method), the tBoc method (t-buthyloxycarbonyl method), or the like, based on the amino acid information thereof. The peptide can be chemically synthesized using a peptide synthesizer (manufactured by Advanced ChemTech, Applied Biosystems, Pharmacia Biotech, Protein Technology Instrument, Synthecell-Vega, PerSeptive, Shimazu, or the like).

The structure of the purified polypeptide of the present invention can be carried out by methods commonly used in protein chemistry (see, for example, Hisashi Hirano. “Protein Structure Analysis for Gene Cloning”, published by Tokyo Kagaku Dojin, 1993). The physiological activity of a polypeptide of the present invention can be measured in accordance with a known measurement method.

Production of a soluble polypeptide useful in the present invention may be achieved by various methods known in the art. For example, the polypeptide may be derived from an intact transmembrane Bmi-1 polypeptide molecule by protein degradation which is carried out by exopeptidase, Edman degradation or a combination of both using specific endopeptidase. The intact Bmi-1 polypeptide molecule may be purified from naturally occurring sources using conventional methods. Alternatively, the intact Bmi-1 polypeptide may be produced by recombinant DNA technology using well known techniques for cDNA, expression vectors, and recombinant gene expression.

Preferably, a soluble polypeptide useful in the present invention may be directly produced. Therefore, the necessity of using the whole Bmi-1 peptide as a starting material is eliminated. This may be achieved by conventional chemical synthesis techniques or well known recombinant DNA techniques (where, expression is carried out in a host in which only a DNA sequence encoding a desired peptide is transformed). For example, a gene encoding a desired soluble Bmi-1 polypeptide may be synthesized by chemical means using an oligonucleotide synthesizer. Such an oligonucleotide is designed based on the amino acid sequence of the desired soluble Bmi-1 polypeptide. A specific DNA sequence encoding a desired peptide may be derived from the full-length DNA sequence by isolation of a specific restriction endonuclease fragment or PCR synthesis of a specific region of cDNA.

(Method for Producing Mutant Polypeptide)

Amino acid deletion, substitution or addition (including fusion) of the polypeptide of the present invention (e.g., Bmi-1 and the like) can be carried out by a site-specific mutagenesis method which is a well known technique. One or several amino acid deletions, substitutions or additions can be carried out in accordance with methods described in Molecular Cloning, A Laboratory Manual, Second Edition, Cold Spring Harbor Laboratory Press (1989); Current Protocols in Molecular Biology, Supplement 1 to 38, John Wiley & Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci USA, 82, 488 (1985); Proc. Natl. Acad. Sci., USA, 81, 5662 (1984); Science, 224, 1431 (1984); PCT WO85/00817 (1985); Nature, 316, 601 (1985); and the like.

(Screening)

As used herein, the term “screening” refers to selection of a target, such as an organism, a substance, or the like, a given specific property of interest from a population containing a number of elements using a specific operation/evaluation method. For screening, an agent (e.g., an antibody), a polypeptide or a nucleic acid molecule of the present invention can be used. Screening may be performed using libraries obtained in vitro, in vivo, or the like (with a system using a real substance) or alternatively in silico (with a system using a computer). It will be understood that the present invention encompasses compounds having desired activity obtained by screening. The present invention is also intended to provide drugs which are produced by computer modeling based on the disclosures of the present invention.

In one embodiment, the present invention provides an assay for screening candidate compounds or test compounds for a protein or polypeptide of the present invention, or a compound capable of binding to a biologically active portion thereof or modulating the activity thereof. The test compounds of the present invention can be obtained using any of the numerous approaches in combinatorial library methods known in the art, including biological libraries, spatially addressable parallel solid phase or solution phase libraries; synthetic library methods requiring deconvolution; the “one-bead one-compound” library method; and synthetic library methods using selection by affinity chromatography The biological library approach is limited to peptide libraries, while the other four approaches are applicable to peptide, non peptide oligomer, or small molecule libraries of compounds (Lam (1997) Anticancer Drug Des. 12: 145).

Examples of methods for the synthesis of molecular libraries can be found in the art as follows: DeWitt et al. (1993) Proc. Natl. Acad. Sci. USA 90: 6909; Erb et al. (1994) Proc. Natl. Acad. Sci. USA 91: 1 11422; Zuckermann et al. (1994) J. Med. Chem. 37: 2678; Cho et al. (1993) Science 261: 1303; Carrell et al. (1994) Angew. Chem. Int Ed. Engl. 33: 2059; Carrell et al. (1994) Angew. Chem. Int. Ed. Engl. 33: 2061; and Gallop et al. (1994) J. Med Chem. 37: 1233.

Libraries of compounds may be presented in solution (e.g., Houghten (1992) Bio Techniques 13: 412-421), or on beads (Lam (1991) Nature 354: 82-84), chips (Fodor (1993) Nature 364: 555-556), bacteria (Ladner, U.S. Pat. No. 5,223,409), spores (Ladner, supra), plasmids (Cull et al. (1992) Proc. Natl. Acad. Sci. USA 89: 1865-1869), or phage (Scott and Smith (1990). Science 249: 386-390; Devlin (1990) Science 249. 404-406; Cwirla et al. (1990) Proc. Natl. Acad. Sci. USA 87: 6378-6382, and Felici (1991) J. Mol. Biol. 222: 301-310; Ladner supra).

(Diseases)

According to an aspect of the present invention, a method for treatment of any disease, disorder or abnormality to which the expansion of stem cells may be applied, such as diseases, disorders or abnormalities of the genital system, nervous diseases, disorders or abnormalities, or hematopoiesis-related diseases, disorders or abnormalities is provided.

Examples of diseases or disorders of the genital system include, but are not limited to, male genital organ diseases (e.g., male sterility, prostatomegaly, prostate cancer, testis cancer, etc.), female genital organ diseases (e.g., female sterility, ovary function disorders, hysteromyoma, adenomyosis uteri, uterine cancer, endometriosis, ovarian cancer, villosity diseases, etc.), and the like. These diseases or disorders can be treated or prevented by expanding genital system stem cells (e.g., sperm stem cells) using the method of the present invention. A method of transplanting sperm stem cells is described in, for example, Brinster R. L., at al., Proc. Natl. Acad. Sci. USA (1994) 91:11298-11302. Specifically, when sperm stem calls are transplanted as donor cells into the testis of a sterile host mouse, the donor stem cells are expanded in the host testis and the progenitors derived from the donor can be obtained. Thus, the present invention can be applied to prevention or treatment of various sterilities.

Examples of hematopoiesis-related diseases include diseases of the circulatory system (e.g., blood cells, etc.). Examples of such diseases or disorders include, but are not limited to, anemia (e.g., aplastic anemia (particularly, severe aplastic anemia), renal anemia, cancerous anemia, secondary anemia, refractory anemia, etc.), cancer or tumor (e.g., leukemia); and after chemotherapy therefor, hematopoietic failure, thrombocytopenia, acute myelocytic leukemia (particularly, a first remission (high-risk group), a second remission and thereafter), acute lymphocytic leukemia (particularly, a first remission, a second remission and thereafter)” chronic myelocytic leukemia (particularly, chronic period, transmigration period), malignant lymphoma (particularly, a first remission (high-risk group), a second remission and thereafter), multiple myeloma (particularly, an early period after the onset), congenital immunodeficiency syndrome, and the like.

The terms “nervous disease” or “neurological disease” are used herein interchangeably to refer to the discontinuation, termination or disorder of a function, a structure, an organ, or the like of a nerve. The term typically refers to a lesion satisfying at least two of the following criteria: 1) the presence of a pathogenic substance; 2) the presence of a symptom and/or a syndrome capable of being clearly indicated; and 3) a corresponding anatomical change. Examples of nervous diseases include, but are not limited to, cerebrovascular disorders (e-g., cerebral hemorrhage, subarachnoid hemorrhage, cerebral infarction, transient (cerebral) ischemic attack (TIA), cerebral arteriosclerosis, Binswanger disease, cerebral sinus thrombosis/cerebral phlebothrombosis, hypertensive encephalopathy, temporal arteritis, transient global amnesia (TGA), moya-moya disease, fibromuscular hyperplasia internal carotid artery/cavernous sinus/fistula, chronic subdural hematoma, amyloid angiopathy (e.g. Alzheimer disease), etc.); circulatory disorder of the spinal cords (e.g., spinal infarct, transient spinal ischemia, spinal hemorrhage, circulatory deformity of the spinal cord, spinal subarachnoid hemorrhage, subacute necrotizing myelitis, etc.); infective and inflammatory disorders (e.g., meningitis, encephalitis, Herpes simplex encephalitis (HSE), Japanese encephalitis, other encephalitises, rabies, slow virus disease (e.g., subacute sclerosing panencephalitis (SSPE), progressive multifocal leukoencephalitis (PML), Creutzfeldt-Jakob disease (CJD), etc.), neural Behcet disease, chorea minor, AIDS dementa syndrome, neuro syphilis, cerebral abscess, spinal epidural abscess, HTLV-1-associated myelopathy (HAM), poliomyelitis; demyelining diseases (multiple sclerosis (MS), acute disseminated encephalomyelitis (ADEM), Balo's concentric sclerosis, inflammatory universal sclerosis, leukodystrophy, metachromatic leukodystrophy, Krabbe's disease, adrenoleukodystrophy (ALD), Canavan's disease (leukodystrophy), Pelizaeus-Merzbacher disease (leukodystrophy), Alexander's disease (leukodystrophy), etc.): dementia disease (Alzheimer's disease, senile dementia of Alzheimer type (SDAT), Pick's disease, cerebrovascular dementia, Creutzfeldt-Jakob disease (CJD), Parkinson-dementia complex, normal pressure hydrocephalus, progressive supranuclear palsy (PSP), etc.); basal nuclei degenerative disease (e.g., Parkinson disease (PD), symptomatic parkinsonism, striatonigral denegeration (SNG), Parkinson-dementia complex, Huntington's disease (HD), essential tremer, athetosis, dystonia syndrome (e.g., idiopathic torsion dystonia, local dystonia (spasmodic wryneck, writer's cramp, Meige's disease, etc.), symptomatic dystonia (Hallervorden-Spats disease, drug-induced dystonia, etc.), Gilles de la Tourette's syndrome, etc.); spinocerebellar degenerative disease (e.g., spinocerebellar degeneration (SCD) (Shy-Drager syndrome, Machado-Joseph disease (MJD), etc.), Louis-Bar syndrome, Bassen-Kornzweig syndrome, Refsum disease, other cerebellar ataxias, etc.); motor neuron diseases (MND) (e.g., amyotrophic lateral sclerosis (ALS), progressive bulbar amytrophy (see amyotrophic lateral sclerosis), familial amyotrophic lateral sclerosis, Werdnig-Hoffmann disease (WHD), Kugelberg-Welander (K-W) disease, bulbar spinal sclerosis, juvenile one upper limb muscular sclerosis, etc.); tumor diseases of brain and spinal cord (e.g., intracranial tumor, spinal abscess, meningeal carcinoma, etc.); functional diseases (e.g., epilepsy, chronic headache, syncope (see syncope), idiopathic endocranial increased infracranial pressure disease, Meniere's disease, narcolepsy, Kleine-Levin syndrome, etc.); toxic and metabolic diseases (e.g., drug intoxication (phenothiazines-derived antipsychotic agent intoxication, sedatives and hypnotics intoxication, antibiotics intoxication, antiparkinson drug intoxication, antitumor drug intoxication, β-blocker intoxication, calcium antagonist intoxication, clofibrate intoxication, antiemetic drug intoxication, SMON disease, salicylic acid intoxication, digitalis intoxication, narcotic addiction, etc.), chronic alcoholism (Wernicke encephalopathy, Marchiafava-Bignami syndrome, central pontine myelinolysis, etc.), organic solvent poisoning and pesticide poisoning (e.g., organophosphate compound poisoning, carbamates poisoning, chloropicrin poisoning, paraquat poisoning, etc.), organophosphate nerve gas poisoning, carbonmonooxide poisoning, hydrogen sulfide poisoning, cyanide compound poisoning, mercurial poisoning (metallic mercurial poisoning, inorganomercurial poisoning, organomercurial poisoning, etc.), lead poisoning, tetraethyl lead poisoning, arsenic poisoning, cadmium poisoning, chrome poisoning, manganese poisoning, metal fume fever, sedatives and hypnotics intoxication, salicylic acid intoxication, digitalis intoxication, narcotic addiction, food poisoning (e.g., natural food poisoning (tetradotoxin poisoning, paralytic shell fish food poisoning, diarrhogenic shell fish food poisoning, ciguatera, mushroom poisoning, potato-plant poisoning, etc.), vitamin deficiency (vitamin A deficiency, vitamin B1 deficiency, vitamin B2 deficiency, pellagra, scurvy, vitamin dependency), lipidosis, Gaucher disease, Niemann-Pick disease, etc.), acquired disorders of amino acid metabolism, Wilson disease, amyloidosis, etc.); congenital deformity (Arnold-Chiari malformation, Klippel-Feil syndrome, basilar impression, syringomyelia); neurosis and dermatopathy (e.g., phacomatosis, von Recklinghausen's disease, tuberous sclerosis, Sturge-Weber syndrome, von Hippel Lindau's disease, etc.); spinal diseases (deformity of the spine herniated intervertebral discs, lateral axial band osteosis, etc.), and the like.

As used herein, the term “nervous disorder” refers to a disorder of a function, structure, or both of a nerve caused hereditarily relating to development, defects in development, or exogenous factors (e.g., toxins, traumas, diseases, etc.). Examples of nervous disorders include, but are not limited to, peripheral nervous disorders, diabetic nervous disorder, and the like. Disorders of the peripheral nerve have various causes. Irrespective of cause, peripheral nervous disorders are collectively called “neuropathy”. Examples of causes for nervous disorders include hereditary, infection, poisoning, metabolic disorders, allergy, collagen diseases, cancer, vascular disorders, traumas, mechanical pressure, tumor, and the like. In some instances no cause for a nervous disorder may be identified clinically. The present invention encompasses nervous disorders having unknown causes as subjects to be treated. Examples of nervous disorders include, but are not limited to, parenchymatous neuropathy and interstitial neuropathy. Parenchymatous neuropathy indicates that at least one of neuron, Schwann cell and medullary sheath which substantially constitute the peripheral nerve are affected by a pathogen, and a lesion occurs therein. Interstitial neuropathy refers to disorders in which stroma is affected. Examples of interstitial neuropathy include, but are not limited to, physical pressure, vascular lesion (periarteritis nodosa (PAN), collagen diseases, etc.), inflammation, and granulation tissue (e.g., leproma, sarcoidosis, etc.). If the metabolism of the whole neuron is disordered, the peripheral portion of a neuron is degenerated; the degeneration progresses toward the cell body; and eventually the nerve cell shrinks (antidromic necrotizing neuropathy), Examples of syndromes of nervous disorders include, but are not limited to, motor disorders, sensory disorders, loss of muscle strength, muscular atrophy, loss of reflex, autonomic disorders, combinations thereof, and the like. The present invention is effective for treatment, prophylaxis and the like of such nervous disorders.

As used herein, the term “nervous condition” refers to the degree of the health of a nerve. Such a condition can be represented by various parameters. The present invention makes it possible to determine the condition of a nerve by measuring Bmi-1 or the like.

As used herein, the term “central nervous system disorder”-refers to any pathological condition associated with abnormal function of the central nervous system (CNS). The term includes, but is not limited to, altered CNS function resulting from physical trauma to cerebral tissue, viral infection, autoimmune mechanism, and genetic mutation.

As used herein, the term “demyelinating disease” refers to a pathological disorder characterized by the degradation of the myelin sheath of the oligodendrocyte cell membrane.

Illustrative examples of diseases, disorders or injuries (conditions) capable of being treated by a molecule or method of the present invention include brain injury, spinal cord injury, stroke, demyelinating diseases (monophasic demyelination), encephalomyelitis, multifocal leukoencephalopathy, panencephalitis, Marchiafava-Bignami disease, Spongy degeneration, Alexander's disease, Canavan's disease, metachromatic leukodystrophy and Krabbe's disease.

As used herein, the terms “prophylaxis”, “prophylactic” and “prevent” refer to the reduction of the possibility that an organism contracts a disease or an abnormal condition occurs in an organism.

As used herein, the terms “treatment” and “treat” refer to a therapeutic effect and partial alleviation or suppression of an abnormal condition of an organism.

As used herein, the term “therapeutic effect” refers to an inhibition or activation agent capable of causing or contributing to an abnormal condition. A therapeutic effect relaxes at least one symptom in an abnormal condition to some extent. A therapeutic effect with reference to the treatment of an abnormal condition may refer to at least one of the following items: (a) increasing the proliferation, growth, and/or differentiation of cells; (b) inhibiting cell death. (i.e., delaying or arresting cell death); (c) inhibiting degeneration; (d) relaxing at least one symptom associated with an abnormal condition; and (e) enhancing the function of an affected cell population. A compound exhibiting efficacy to an abnormal condition may be identified as described herein.

As used herein, the term “abnormal condition” refers to a function of a cell or tissue of an organism which departs from the normal condition. An abnormal condition may be associated with cell proliferation, cell differentiation, cell signal transduction, or cell survival. An abnormal condition may also include an abnormality in nerve transmission, obesity, diabetic complication (e.g., retina degeneration), irregular glucose intake or metabolism, and irregular fatty acid intake or metabolism.

Examples of abnormal cell proliferation include cancer, neoplasm, tumor, inflammation, and the like.

Examples of abnormal differentiation include malformation, Cancer, and the like.

Examples of abnormal cell signal transduction include abnormal cell differentiation, and the like.

Abnormal cell survival is related to activation or suppression of an apoptosis (programmed cell death) pathway. A number of protein kinases are associated with the apoptosis pathway. An abnormality in a function of one of the protein kinases may lead to the immortality of a cell or death of immature cells.

In another aspect, the present invention provides both a prophylactic method and a therapeutic method for treating a subject having (or suspected of having) a nervous disease, disorder or abnormal condition, or hematopoiesis-related diseases, disorders or abnormalities, or a subject having the above-described disorders.

Diseases and disorders that are characterized by increased (relative to a subject not suffering from the disease or disorder) levels of biological activity may be treated with therapeutics that antagonize (i.e., reduce or inhibit) activity. Therapeutics that antagonize activity may be administered in a therapeutic or prophylactic manner. Therapeutics that may be utilized include, but are not limited to, (i) Bmi-1 (e.g., a polypeptide), or analogs, derivatives, fragments or homologs thereof; (ii) antibodies to Bmi-1; (iii) nucleic acids encoding Bmi-1 (where the agent is a polypeptide); (iv) administration of antisense nucleic acid and nucleic acids that are “dysfunctional” (i.e., due to a heterologous insertion within the coding sequences of coding sequences for Bmi-1 (polypeptide)) (e g. RNAi) are utilized to “knockout” endogenous function of Bmi-1 by homologous recombination (see, e.g., Capecchi (1989) Science 244; 1288-1292); or (v) modulators (i.e., inhibitors, agonists and antagonists, including additional peptide mimetics of the present invention or antibodies specific to a peptide of the present invention) that modulates the interaction between Bmi-1 and its binding partner.

Diseases and disorders that are characterized by decreased (relative to a subject not suffering from the disease or disorder) levels of biological activity may be treated with therapeutics that increase (i.e., are agonists to) activity. Therapeutics that upregulate activity may be administered in a therapeutic or prophylactic manner. Therapeutics that may be utilized include, but are not limited to, Bmi-1, or analogs, derivatives, fragments or homologs thereof; or an agonist that increases bioavailability.

Increased or decreased levels can be readily detected by quantifying peptide and/or RNA, by obtaining a patient's tissue sample (e.g., from biopsy tissue) and assaying it in vitro for RNA or peptide levels, structure and/or activity of the expressed peptides (or mRNAs of Bmi-1). Methods that are well known in the art include, but are not limited to, immunoassays (e.g., by Western blot analysis, immuno precipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

The present invention provides a method for preventing abnormal expression of Bmi-1 or a disease or condition associated with the activity of Bmi-1 by administering a drug capable of modulating the expression of Bmi-1 or the activity of Bmi-1. A subject at a risk of a disease caused or contributed by abnormal expression of Bmi-1 or the activity of Bmi-1, may be identified using either a diagnosis assay or a prognosis assay as described herein or a combination thereof. A prophylactic agent may be administered before appearance of a symptom characteristic to an abnormality in Bmi-1. As a result, a disease or disorder can be prevented or its progression delayed. In accordance with the type of an abnormality in Bmi-1, for example, an agonist or antagonist agent for Bmi-1 may be used to treat a subject. An appropriate drug may be determined based on screening assays described herein.

The present invention also relates to a method for modulating the expression or activity of Bmi-1 for therapeutic purposes. The modulation method of the present invention comprises a step of contacting cells with a drug capable of modulating at least one activity of Bmi-1 associated with the cell. A drug for modulating the activity of Bmi-1 may be a drug as described herein, such as a nucleic acid or a protein, naturally-occurring cognate ligands and peptides of Bmi-1, peptide mimics of Bmi-1, or other small molecules. In one embodiment, a drug may stimulate at least one Bmi-1 activity. Examples of such a stimulant include a nucleic acid encoding active Bmi-1 and a nucleic acid encoding Bmi-1, which is introduced into cells. In another embodiment, a drug inhibits at least one Bmi-1 activity. Examples of such an inhibitor include an antisense strand for a nucleic acid encoding Bmi-1 and an antibody against Bmi-1. These modulation methods may be carried out in vitro (e.g., culturing cells with a drug) or in vivo (e.g., administering a drug into a subject). Thus, the present invention provides a method for treating a subject suffering from a disease or disorder characterized by the abnormal expression or abnormal activity of a nucleic acid molecule encoding Bmi-1 (e.g., a polypeptide), In one embodiment, the method comprises a step of administering a combination of a drug (e.g., a drug identified by a screening assay described herein) and a drug capable of modulating (e.g., upregulating or downregulating) the expression or activity of Bmi-1. In another embodiment, the method comprises a step of administering Bmi-1 or a nucleic acid molecule encoding it in order to compensate for reduced or abnormal expression or activity of Bmi-1.

(Gene Therapy)

In a specific embodiment, a nucleic acid containing the nucleic acid sequence of a normal gene of the present invention, or a sequence encoding an antibody or a functional derivative thereof is administered for the purposes of gene therapy for treating, inhibiting, or preventing diseases or disorders associated with the abnormal expression and/or activity of a polypeptide of the present invention. Gene therapy refers to a therapy performed by administrating a nucleic acid, which has been expressed or is capable of being expressed, into subjects. In this embodiment of the present invention, a nucleic acid produces a protein encoded thereby and the protein mediates a therapeutic effect.

Any method available in the art for gene therapy may be used in accordance with the present invention. Illustrative methods are described below.

See the following review articles for gene therapy: Goldspiel et al., Clinical Pharmacy 12:488-505 (1993); Wu and Wu, Biotherapy 3:87-95 (1991); Tolstoshev, Ann. Rev. Pharmacol. Toxicol., 32:573-596 (1993); Mulligan, Science 260:926-932 (1993); Morgan and Anderson, Ann. Rev. Biochem. 62:191-217 (1993); and May, TIBTECH 11(5):155-215(1993). Generally known recombinant DNA techniques used for gene therapy are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

Therefore, in the present invention, gene therapy using a nucleic acid molecule encoding Bmi-1, or a variant or fragment thereof, or the like may be useful.

As used herein, the terms “trait” and “phenotype” are used interchangeably to refer to an observable trait, a detectable trait or other measurable traits of organisms. An example of a trait is a symptom of a disease or sensitivity to a disease. The term “trait” or “phenotype” may be used herein typically to refer to symptoms of nervous diseases, disorders or abnormalities, or hematopoiesis-related diseases, disorders or abnormality, or the morbidity thereof.

As used herein, the term “genotype” refers to a genetic structure of an individual organism, and often referees to an allele present in an individual or sample. The term “determine the genotype” of a sample or individual encompasses analysis of the sequence of a specific gene of the individual.

As used herein, the term “polymorphism” refers to the occurrence of at least two selective genomic sequences or alleles between different genomes or individuals. The term “polymorphism (polymorphic)” refers to a state having the possibility that at least two mutants are found in a specific genomic sequence in individuals. The term “polymorphic site” refers to a gene locus at which such a mutation occurs. Single nucleotide polymorphisms (SNPs) indicate that a nucleotide is replaced with another nucleotide at a polymorphic site. A single nucleotide deletion or insertion can lead to a single nucleotide polymorphism. As used herein, the term “single nucleotide polymorphism” preferably refers to a single nucleotide substitution. In general, two different nucleotides may share a polymorphic site between different individuals, In the present invention, polymorphisms of Bmi-1, and the like are considered to be associated with nervous diseases. In one embodiment, alleles identified by such polymorphism analysis may be effective for regeneration, prophylaxis, diagnosis, treatment, or prognosis.

As used herein, the term “synthesis” or “synthesize” refers to a chemical substance (e.g., a polynucleotide, a polypeptide, or the like) which is purely chemically produced in contrast to enzymatic methods. Therefore, a “globally” synthesized chemical substance (e.g., a polynucleotide, a polypeptide, or the like) includes one that is globally produced by chemical means, while a “partially” synthesized chemical substance (e.g., a polynucleotide, a polypeptide, or the like) includes one that is only partially produced by chemical means.

As used herein, the term “region” refers to a physically contiguous portion of the primary structure of a biomolecule. In the case of a protein, a region is defined by a portion having a contiguous amino acid sequence. As used herein, the term “domain” refers to a structural portion of a biomolecule which contributes to a known or inferred function of the biomolecule. A domain may have the same range as a region or a portion thereof. A domain may comprise a portion of a biomolecule, which is distinguished from a specific region, in addition to the whole or a part of the region. Examples of a domain of a protein in Bmi-1 according to the present invention include, but are not limited to, a signal peptide, an extracellular (i.e., N-terminal) domain, and a leucine rich repeat domain.

(Demonstration of Therapeutic Activity or Prophylactic Activity)

The compounds or pharmaceutical compositions of the present invention are preferably tested in vitro, and then in vivo for the desired therapeutic or prophylactic activity (e.g., nervous diseases, disorders or abnormality, or hematopoiesis-related diseases, disorders or abnormalities, and the like), prior to use in humans. For example, in vitro assays to demonstrate the therapeutic or prophylacticutility of a compound or pharmaceutical composition include, the effect of a compound on a cell line or a patient tissue sample. The effect of the compound or composition on the cell line and/or tissue sample can be determined utilizing techniques known to those of skill in the art (including, but not limited to, cell lysis assays). In accordance with the present invention, in vitro assays which can be used to determine whether administration of a specific compound is indicated, include in vitro cell culture assays in which a patient tissue sample is grown in culture, and exposed to or otherwise administered a compound, and the effect of such a compound upon the tissue sample is observed.

(Administration and Composition for Regeneration/Therapy/Prophylaxis)

The present invention provides methods of treatment, inhibition and prophylaxis of nervous diseases, disorders or abnormality, or hematopoiesis-related diseases, disorders or abnormality, by administration to a subject a compound or pharmaceutical composition of the present invention in an effective amount. In a preferred aspect, the compound is substantially purified (e.g., substantially free from substances that limit its effect or produce undesired side-effects).

As used herein, the term “amount effective for diagnosis, prophylaxis, treatment, or prognosis” refers to an amount which is recognized as therapeutically effective for diagnosis, prophylaxis, treatment (or therapy), or prognosis. Such an amount can be determined by those skilled in the art using techniques well known in the art with reference to various parameters.

Animals targeted by the present invention include any organism as long as it has a nervous system or an analogous system (e.g., animals (e.g., vertebrates, invertebrate)). Preferably, the animal is a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialia, edentate, dermoptera, chiroptera, carnivore, insectivore, proboscidea, perissodactyla, artidactyla, tubulidentata, pholidota, sirenia, cetacean, primates, rodentia, lagomorpha, etc.). Illustrative examples of a subject include, but are not limited to, animals, such as cattle, pig, horse, chicken, cat, dog, and the like. More preferably, cells derived from Primates (e.g., chimpanzee, Japanese monkey, human) are used. Most preferably, cells derived from a human are used.

When a nucleic acid molecule or polypeptide of the present invention is used as a medicament, the medicament may further comprise a pharmaceutically acceptable carrier. Any pharmaceutically acceptable carrier known in the art may be used in the medicament of the present invention.

Examples of a pharmaceutical acceptable carrier or a suitable formulation material include, but are not limited to, antioxidants, preservatives, colorants, flavoring agents, diluents, emulsifiers., suspending agents, solvents, fillers, bulky agents, buffers, delivery vehicles, and/or pharmaceutical adjuvants. Representatively, a medicament of the present invention is administered in the form of a composition comprising a polypeptide or a polynucleotide, such as Bmi-1, or a variant or fragment thereof, or a variant or derivative thereof with at least one physiologically acceptable carrier, excipient or diluent. For example, an appropriate vehicle may be injection solution, physiological solution, or artificial cerebrospinal fluid, which can be supplemented with other substances which are commonly used for compositions for parenteral delivery.

Acceptable carriers, excipients or stabilizers used herein preferably are nontoxic to recipients and are preferably inert at the dosages and concentrations employed, and preferably include phosphate, citrate, or other organic acids; ascorbic acid, α-tocopherol; low molecular weight polypeptides; proteins (e.g., serum albumin, gelatin, or immunoglobulins); hydrophilic polymers (e.g., polyvinyl pyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor.

Hereinafter, commonly used preparation methods of the medicament of the present invention will be described. Note that animal drug compositions, quasi-drugs, marine drug compositions, food compositions, cosmetic compositions, and the like can be prepared using known preparation methods.

The polypeptide, polynucleotide and the like of the present invention can be mixed with a pharmaceutically acceptable carrier and can be orally or parenterally administered as solid formulations (e.g., tablets, capsules, granules, abstracts, powders, suppositories, etc.) or liquid formulations (e.g., syrups, injections, suspensions, solutions, spray agents, etc.). Examples of pharmaceutically acceptable carriers include excipients, lubricants, binders, disintegrants, disintegration inhibitors, absorption promoters, adsorbers, moisturizing agents, solubilizing agents, stabilizers and the like in solid formulations; and solvents, solubilizing agents, suspending agents, isotonic agents, buffers, soothing agents and the like in liquid formulations. Additives for formulations, such as antiseptics, antioxidants, colorants, sweeteners, and the like can be optionally used. The composition of the present invention can be mixed with substances other than the polynucleotides, polypeptides, and the like of the present invention. Examples of parenteral routes of administration include, but are not limited to, intravenous injection, intramuscular injection, intranasal, rectum, vagina, transdermal, and the like.

Examples of excipients in solid formulations include glucose, lactose, sucrose, D-mannitol, crystallized cellulose, starch, calcium carbonate, light silicic acid anhydride, sodium chloride, kaolin, urea, and the like.

Examples of lubricants in solid formulations include, but are not limited to, magnesium stearate, calcium stearate, boric acid powder, colloidal silica, talc, polyethylene glycol, and the like.

Examples of binders in solid formulations include, but are not limited to, water, ethanol, propanol, saccharose, D-mannitol, crystallized cellulose, dextran, methylcellulose, hydroxypropylcellulose, hydroxypropyl methylcellulose, carboxymethylcellulose, starch solution, gelatin solution, polyvinylpyrrolidone, calcium phosphate, potassium phosphate, shellac, and the like.

Examples of disintegrants in solid formulations include, but are not limited to, starch, carboxymethylcellulose, carboxymethylcellulose calcium, agar powder, laminarin powder, croscarmellose sodium, carboxymethyl starch sodium, sodium alginate, sodium hydrocarbonate, calcium carbonate, polyoxyethylene sorbitan fatty acid esters, sodium lauryl sulfate, starch, monoglyceride stearate, lactose, calcium glycolate cellulose, and the like.

Examples of disintegration inhibitors in solid formulations include, but are not limited to, hydrogen-added oil, saccharose, stearin, cacao butter, hydrogenated oil, and the like.

Examples of absorption promoters in solid formulations include, but are not limited to, quaternary ammonium salts, sodium lauryl sulfate, and the like.

Examples of absorbers in solid formulations include, but are not limited to, starch, lactose, kaolin, bentonite, colloidal silica, and the like.

Examples of moisturizing agents in solid formulations include, but are not limited to, glycerin, starch, and the like.

Examples of solubilizing agents in solid formulations include, but are not limited to, arginine, glutamic acid, aspartic acid, and the like.

Examples of stabilizers in solid formulations include, but are not limited to, human serum albumin, lactose, and the like.

When tablets, pills, and the like are prepared as solid formulations, they may be optionally coated with a film of a substance dissolvable in the stomach or the intestine (saccharose, gelatin, hydroxypropylcellulose, hydroxypropylmethylcellulose phthalate, etc.). Tablets include those optionally with a typical coating (e.g., dragees, gelatin coated tablets, enteric coated tablets, film coated tablets or double tablets, multilayer tablets, etc.). Capsules include hard capsules and soft capsules. When tablets are molded into the form of a suppository, higher alcohols, higher alcohol esters, semi-synthesized glycerides, or the like can be added in addition to the above-described additives. The present invention is not so limited.

Preferable examples of solutions in liquid formulations include injection solutions, alcohols, propyleneglycol, macrogol, sesame oil, corn oil, and the like.

Preferable examples of solubilizing agents in liquid formulations include, but are not limited to, polyethyleneglycol, propyleneglycol, D-mannitol, benzyl benzoate, ethanol, trisaminomethane, cholesterol, triethanolamine, sodium carbonate, sodium citrate, and the like.

Preferable examples of suspending agents in liquid formulations include surfactants (e.g., stearyltriethanolamine, sodium lauryl sulfate, lauryl amino propionic acid, lecithin, benzalkonium chloride, benzethonium chloride, glycerin monostearate, etc.), hydrophilic macromolecule (e.g., polyvinyl alcohol, polyvinylpyrrolidone, carboxymethylcellulose sodium, methylcellulose, hydroxymethylcellulose, hydroxyethylcellulose, hydroxypropylcellulose, etc.), and the like. Preferable examples of isotonic agents in liquid formulations include, but are not limited to, sodium chloride, glycerin, D-mannitol, and the like.

Preferable examples of buffers in liquid formulations include, but are not limited to, phosphate, acetate, carbonate, citrate, and the like.

Preferable examples of soothing agents in liquid formulations include, but are not limited to, benzyl alcohol, benzalkonium chloride, procaine hydrochloride, and the like.

Preferable examples of antiseptics in liquid formulations include, but are not limited to, parahydroxybenzoate ester, chlorobutanol, benzyl alcohol, 2-phenylethylalcohol, dehydroacetic acid, sorbic acid, and the like.

Preferable examples of antioxidants in liquid formulations include, but are not limited to, sulfite, ascorbic acid, α-tocopherol, cysteine, and the like.

When liquid agents and suspensions are prepared as injections, they are sterilized and are preferably isotonic with the blood. Typically, these agents are made aseptic by filtration using a bacteria-retaining filter or the like, mixing with a bactericide or, irradiation, or the like. Following these treatment, these agents may be made solid by lyophilization or the like. Immediately before use, sterile water or sterile injection diluent (lidocaine hydrochloride aqueous solution, physiological saline, glucose aqueous solution, ethanol or a mixture solution thereof, etc.) may be added.

The pharmaceutical composition of the present invention may further comprise a colorant, a preservative, a flavor, an aroma chemical, a sweetener, or other drugs.

The medicament of the present invention may be administered orally or parenterally. Alternatively, the medicament of the present invention may be administered intravenously or subcutaneously. When systemically administered, the medicament for use in the present invention may be in the form of a pyrogen-free, pharmaceutically acceptable aqueous solution. The preparation of such pharmaceutically acceptable compositions, with due regard to pH, is isotonicity, stability and the like, is within the skill of the art. Administration methods may herein include oral administration and parenteral administration (e.g., intravenous, intramuscular, subcutaneous, intradermal, mucosal, intrarectal, vaginal, topical to an affected site, to the skin, etc.). A prescription for such administration may be provided in any formulation form. Such a formulation form includes liquid formulations, injections, sustained preparations, and the like.

The medicament of the present invention may be prepared for storage by mixing a sugar chain composition having the desired degree of purity with optional physiologically acceptable carriers, excipients, or stabilizers (Japanese Pharmacopeia ver. 14 or the latest version; Remington's Pharmaceutical Sciences, 18th Edition, A. R. Gennaro, ed., Mack Publishing Company, 1990; and the like), in the form of lyophilized cake or aqueous solutions.

Various delivery systems are known and can be used to administer a compound of the present invention (e.g., liposomes, microparticles, microcapsules). Methods of introduction include, but are not limited to, intradermal, intramuscular, intraperitoneal, intravenous, subcutaneous, intranasal, epidural, and oral routes. The compounds or compositions may be administered by any convenient route (e.g., by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the present invention into the central nervous system by any suitable route (including intraventricular and intrathecal injection; intraventricular injection may be facilitated by an intraventricular catheter, for example, attached to a reservoir, such as an Ommaya reservoir). Pulmonary administration can also be employed, e.g., by use of an inhaler or nebulizer, and formulation with an aerosolizing agent.

In a specific embodiment, it may be desirable to administer a polypeptide, polynucleotide or composition of the present invention locally to the area in need of treatment (e.g., the central nervous system, the brain, etc.); this may be achieved by, for example, and not by way of limitation, local infusion during surgery, topical application (e.g., in conjunction with a wound dressing after surgery), by injection, by means of a catheter, by means of a suppository, or by means of a transplant (the transplant being of a porous, non-porous, or gelatinous material, including membranes, such as sialastic membranes, or fibers). Preferably, when administering a protein, including an antibody, of the present invention, care must be taken to use materials to which the protein does not absorb.

In another embodiment, the compound or composition can be delivered in a vesicle, in particular a liposome (see Langer, Science 249: 1527-1533 (1990): Treat et al., Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14: 201 (1987); Buchwald et al., Surgery 88: 507 (1980); Saudek et al., N. Engl. J. Med. 321: 574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (L984); Ranger and Peppas, J., Macromol. Sci. Rev. Macromol. Chem. 23: 61 (1983); see also Levy et al., Science 228: 190 (1985); During et al., Ann. Neurol. 25: 351 (1989); Howard et al., J. Neurosurg. 71:105 (1989)).

In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249: 1527-1533 (1990)).

The amount of a compound used in the treatment method of the present invention can be easily determined by those skilled in the art with reference to the purpose of use, target disease (type, severity, and the like), the patient's age, weight, sex, and case history, the form or type of the cells, and the like. The frequency of the treatment method of the present invention which is applied to a subject (patient) is also determined by the those skilled in the art with respect to the purpose of use, target disease (type, severity, and the like), the patient's age, weight, sex, and case history, the progression of the therapy, and the like. Examples of the frequency include once per day to once per several months (e.g., once per week to once per month). Preferably, administration is performed once per week to once per month with reference to the progression.

The doses of the polypeptides, polynucleotides or the like of the present invention vary depending on the subjects age, weight and condition or administration method, or the like, including, but not limited to, ordinarily 0.01 mg to 10 g per day for an adult in the case of oral administration, preferably 0.1 mg to 1 g, 0.1 mg to 10 mg, 1 mg to 100 mg, and the like; in the parenteral administration, 0.01 mg to 1 g, preferably 0.01 mg to 100 mg, 0.1 mg to 100 mg, 0.1 mg to 10 mg, 1 mg to 100 mg, and the like. The present invention is not limited to a particular dose.

As used herein, the term “administer” means that the polypeptides, polynucleotides or the like of the present invention or pharmaceutical compositions containing them are incorporated into cell tissue of an organism either alone or in combination with other therapeutic agents. Combinations may be administered either concomitantly (e.g., as an admixture), separately but simultaneously or concurrently; or sequentially. This includes presentations in which the combined agents are administered together as a therapeutic mixture, and also procedures in which the combined agents are administered separately but simultaneously (e.g., as through separate intravenous lines into the same individual). “Combination” administration further includes the separate administration of one of the compounds or agents given first, followed by the second.

Abnormal conditions may be prevented or treated by administering a compound into cells having abnormality in a signal transduction pathway for an organism and then monitoring an effect of the administration of the compound on a biological function. The organism is preferably a mouse, a rat, a rabbit, or a goat, more preferably a monkey or an ape, and most preferably a human.

As used herein, “instructions” describe a method of administering a medicament of the present invention, a method for diagnosis, or the like for persons who administer, or are administered the medicament, the medicament or the like or persons who diagnose or are diagnosed (e g., physicians, patients, and the like). The instructions provide a statement indicating an appropriate method for administrating a diagnostic, medicament, or the like of the present invention. The instructions are prepared in accordance with a format defined by an authority of a country in which the present invention is practiced (e.g., Health, Labor and Welfare Ministry in Japan, Food and Drug Administration (FDA) in U.S., and the like), explicitly describing that the instructions are approved by the authority. The instructions are a so-called package insert and are typically provided in paper media. The instructions are not so limited and may be provided in the form of electronic media (e g., web sites and electronic mails provided on the Internet).

The judgment of termination of treatment with a method of the present invention may be supported by a result of a standard clinical laboratory using commercially available assays or instruments or extinction of a clinical symptom characteristic to a disease (e.g., a nervous disease) associated with Bmi-1, or the like. Treatment can be resumed with the relapse of a disease (e.g., a nervous disease) associated with Bmi-1, or the like.

The present invention also provides a pharmaceutical package or kit comprising one or more containers loaded with one or more pharmaceutical compositions. A notice in a form defined by a government agency which regulates the production, use or sale of pharmaceutical products or biological products may be arbitrarily attached to such a container, representing the approval of the government agency relating to production, use or sale with respect to administration to humans.

The plasma half-life and internal body distribution of a drug or a metabolite in the plasma, tumor and major organs may be determined so as to facilitate the selection of the most appropriate drug for inhibiting disorders. Such a measurement may be carried out by, for example, HPLC analysis of the plasma of an animal treated by a drug. The location of a radiolabeled compound may be determined using a detection method, such as X-ray, CAT scan, or MRI. A compound which exhibits strong inhibition activity in screening assays but has insufficient pharamacokinetic characteristics may be optimized by changing or retesting the chemical structure thereof. In this regard, a compound having satisfactory pharmacokinetic characteristics may be used as a model.

Toxicity studies may be carried out by measuring blood cell composition. For example, a toxicity study may be carried out in the following appropriate animal model: (1) a compound is administered into mice (an untreated control mouse should also be used); (2) a blood sample is periodically obtained from a mouse in each treatment group via the tail vein; and (3) the sample is analyzed for the numbers of erythrocytes and leukocytes, the blood cell composition, and the ratio of lymphocytes and polymorphonuclear cells. Comparison of the result of each drug regimen with the control shows whether or not toxicity is present.

At the end of each toxicity study, a further study may be carried out by sacrificing the animal (preferably, in accordance with American Veterinary Medical Association guidelines Report of the American Veterinary Medical Assoc. Panel on Euthanasia, (1993) J. Am. Vet. Med. Assoc. 202: 229-249). Thereafter, a representative animal from each treatment group may be tested by viewing the whole body for direct evidence of transitions, abnormal diseases or toxicity. A gross abnormality in tissue is described and the tissue is hisotologically tested. A compound causing a reduction in weight or a reduction in blood components is not preferable as are compounds having an adverse action to major organs. In general, the greater the adverse action, the less preferable the compound.

(Expansion of Hematopoletic Cells)

Hematopoletic cells are produced in bone marrow, and are differentiated into erythrocytes, platelets, leukocytes, and the like which in turn enter the peripheral blood. Bone marrow cells are originated from pluripotent stem cells which are differentiated into hematopoietic stem cells specialized in the production of blood cells. The specialized cell is differentiated into pluripotent precursor cells which are in turn differentiated into myelocyte precursor cells and lymphocyte precursor cells.

In the myelocyte system, the pluripotent stem cell is differentiated into CFU-GEMM cells. A CFU-GEM cell is differentiated into a CFU-GM cell, a myeloblast, a promyelocyte, and a myelocyte in sequence. These cells are present within the bone marrow, and are differentiated into neutrophils which enter the peripheral blood. In another line, the CFU-GM cell is differentiated into a monoblast, a promonocyte, and a monocyte in sequence. The monocyte migrates to the peripheral blood. In a third line, the CFU-GEMM cell is differentiated into a BFU-E cell, a proerythroblast, an erythroblast, and an erythrocyte in sequence. Additionally, in a megakaryocyte system, the stem cell is differentiated into a CFU-Meg (the abbreviation of megakaryocyte), a megakaryocytoblast, a megakaryocyte, and a platelet in sequence.

An abnormality in the pluripotent stem cell during the differentiation is responsible for leukemia. Since the present invention can eliminate such an abnormality, the present invention may be applied to the treatment of leukemia.

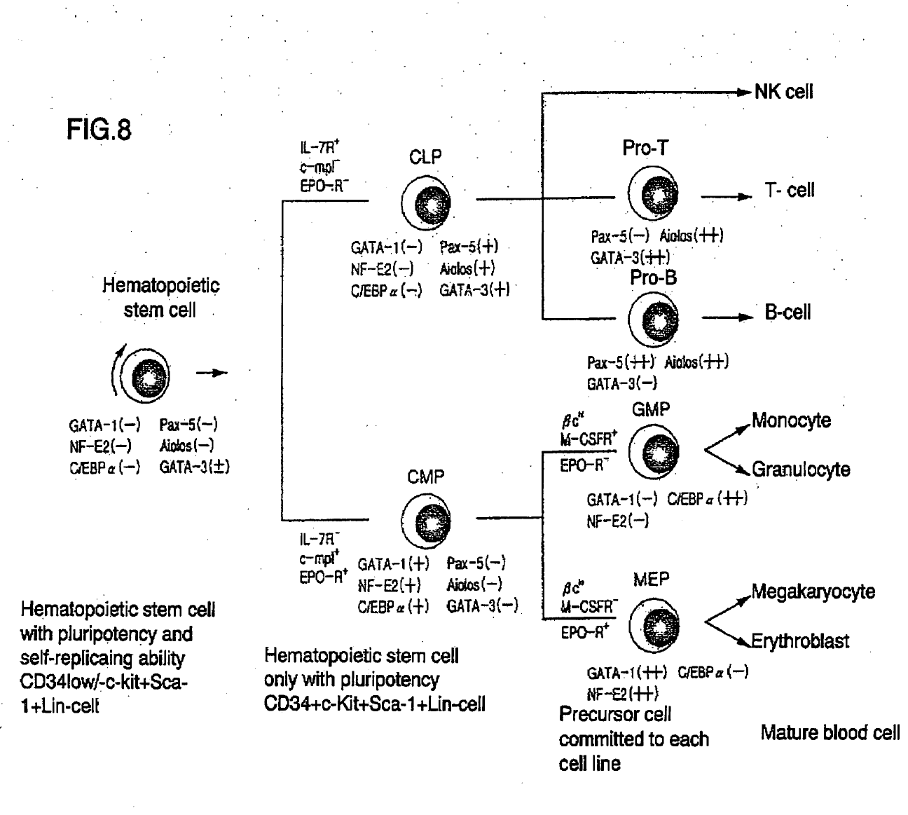

In the lymphocyte system, the pluripotent stem cell is differentiated into a lymphocyte stem cell, which is divided into a B-cell line and a T-cell line. In an additional line, the lymphocyte stem cell is differentiated into an NK cell. In the B-cell line, the stem cell is differentiated into a pro-B-cell, a pre-B-cell, an early B-cell and the like, an intermediate B-cell, a mature B-cell, a plasmacytoid B-cell, a plasma B-cell in sequence. In the T-cell line, the stem cell is differentiated into a precursor thymocyte, an immature thymocyte, a common thymocyte, and a mature thymocyte in sequence. The mature thymocyte is composed of two types of T cells, a helper/inducer T cell and a suppressor/cytotoxic T cells. Therefore, an agent or composition of the present invention may be effective for the treatment or prophylaxis of abnormality in T-cells and/or B-cells. FIG. 8 is a schematic diagram showing the above-described differentiation scheme. In FIG. 8, markers useful for differentiation are described. For detailed description of the differentiation, see, Koichi Akashi, Saishin Igaku [Recent Medicine], 56(2), 15-23, 2001, which is herein incorporated by reference.

As used herein, differentiated cells are represented by the following abbreviations.
n: neutrophil
m: macrophage
e: eosinophil
mast: mast cell
M: megakaryocyte
E: erythrocyte
GM: granulocyte/macrophage
GEM: granulocyte/erythrocyte/macrophage
GMM: granulocyte/macrophage/megakaryocyte
GEMM: granulocyte/erythrocyte/macrophage/megakaryocyte

(Methods for Identifying Hematopoietic Stem Cell)

Hereinafter, representative methods for identifying hematopoietic stem cells will be described.

a. Spleen Colony Method

Mice are exposed to a lethal dose of radiation. Hematopoietic cells from syngeneic mice are intravenously injected into the radiated mice. Bumps (colonies) are observed on the spleen surface 8 to 14 days after injection. Each colony is composed of various blood cells, however, a single colony is derived from a single stem cell. The mother cell which forms the spleen colony is called CFU-S (colony forming unit in spleen). The spleen colony on day 8 (Day 8 CFU-S) was mostly composed of erythroblasts, while the spleen colony on day 12 (Day 12 CFU-S) contained granulocytes or megakaryocytes in addition to erythroblasts. Day 12 CFU-S also contained B-lymphocytes. The spleen colony had a high level of proliferation ability and was derived from a pluripotent stem cell. Day 12 CFU-S is used as a measure of a pluripotent stem cell. A hematopoietic stem cell, which survives after 5-fluoro-uracil (5-FU: an antitumor agent) is administered thereinto, has a considerably high level of proliferation ability. The mother cell of CFU-S is called pre-CFU-S. Day 12 CFU-S was considered to serve as a measure of a hematopoietic stem cell. Actually, Day 12 CFU-S is a non-uniform cell population, and therefore, cannot be necessarily used as a measure of an immature hematopoietic stem cell.

b. Long-Term Bone Marrow Reconstruction Method

In this method, mice are subjected to a lethal dose of radiation; the hematopoietic systems of the mice are reconstructed; and it is observed whether or not the system can be maintained over a long term. At present, this method is the most reliable for determination of the pluripotency and self-replication ability of a hematopoietic stem cell. As a marker, the expression of a neomycin-resistant gene, male and female sex chromosomes, a congenic mouse, or the like is employed. In this method, it was difficult to achieve quantitation. Recently, a competitive repopulation assay has been employed, in which a recipient's hematopoietic cells as well as a donor's hematopoietic cells are transplanted and the rate of the reconstruction thereof is investigated. Since it is difficult to produce an in vivo transplantation experimental system for a human unlike a mouse, a Scid-hu mouse is employed in which a human hematopoietic stem cell is transplanted in an immunodeficient mouse (SCID mouse) which has no rejection reaction because of lack of lymphocytes. In this system, a human hematopoietic system can be maintained in a mouse over a long term.

c. In Vitro Colony Method

In this method, a hematopoietic cell (a bone marrow cell, a spleen cell, or the like) is cultured in a semi-solid medium, such as methyl cellulose, soft agar, or the like, in the presence of various cytokines, and based on the cell populations (colonies) formed, the number or nature of hematopoietic stem cells are estimated. By analyzing the colonies, the differentiation and proliferation processes of various precursor hematopoietic cells or hematopoietic stem cells can be observed or measured in vitro. Mixed colonies (CFU-Mix, CFU-GEMM) or highly pluripotent colonies (HPP-CFC; high proliferative potential colony forming cells) are believed to be more undifferentiated than cells (CFU-GM, BFU-E, etc.) forming a single-line colony. Blast colony forming cell (CFU-blast) is believed to be the most undifferentiated. With this method, the proliferation and differentiation processes of hematopoietic stem cells or the precursor cells thereof can be investigated in vitro. Recently, by using a serum-free medium or single cell culture, the actions of various cytokines involved in hematopoiesis can be estimated.

d. Coculture system with Stroma Cell

Microscopic environments are closely involved in the differentiation and proliferation of hematopoietic stem cells. In 1977, Dexter et al. demonstrated that hematopoietic stem cells can be cultured on bone marrow stromal cells over a long term of several months or more (Dexter's culture method). Thereafter, stromal cell lines capable of maintaining hematopoietic cells have been established one after another. Thus, microscopic environments for hematopoiesis can be reproduced in vitro. In this culture system, whereas precursor hematopoietic cells lose colony forming ability early, undifferentiated hematopoietic stem cells can maintain colony forming ability or bone marrow reconstitution ability over a long term. Therefore, the method is also employed for measurement of the activity of undifferentiated hematopoietic stem cells. Especially for a human, since it is difficult to use an in vivo system, cells (Long term culture-initiating cells: LTC-IC), which can maintain colony forming ability over a long term on stromal cells, are employed as a measure of undifferentiated hematopoietic stem cells.

### BEST MODE FOR CARRYING OUT THE INVENTION

Hereinafter, preferred embodiments of the present invention will be described. The following embodiments are provided for a better understanding of the present invention and the scope of the present invention should not be limited to the following description. It will be clearly appreciated by those skilled in the art that variations and modifications can be made without departing from the scope of the present invention with reference to the specification.

According to an aspect of the present invention, a method for regulating the expansion of a hematopoietic stem cell is provided. The method comprises the steps of: (A) providing, to the hematopoietic stem cell, Bmi-1 or a variant or fragment thereof and/or a Bmi-1 regulating agent in an amount sufficient for regulation of the expansion of the hematopoietic stem cell; and (B) culturing the hematopoietic stem cell for a time sufficient for regulation of the expansion. A technique for providing, to a hematopoietic stem cell, Bmi-1 or a variant or fragment thereof and/or a Bmi-1 regulating agent is well known in the art. For example, the technique comprises culturing a cell in a medium and providing the agent to the medium. The present invention is not limited to this. An amount sufficient for regulation of the expansion can be appropriately determined by those skilled in the art. Hematopoletic stem cells can be cultured using common techniques well known in the art, A time sufficient for regulation of the expansion can be appropriately determined by those skilled in the art in view of the present specification.

Therefore, according to another aspect of the present invention, a composition for regulating the expansion of a hematopoietic stem cell is provided, which comprises Bmi-1 or a variant or fragment thereof and/or a Bmi-1 regulating agent in an amount sufficient for regulation of the expansion. Preferably, the composition may be a pharmaceutical composition or an agricultural composition, In this case, the composition may comprise a pharmaceutically or agriculturally acceptable carrier.

Preferably, the present invention provides a composition and method for promoting the expansion of a stem cell (e.g., a hematopoietic stem cell). No method for efficiently promoting the expansion of a stem cell, such as a hematopoietic stem cell or the like, has heretofore been known. The present invention provides a remarkable effect in the art. As used herein, the term “promotion of expansion” refers to promotion of the self replication of a stem cell. The term “promotion of expansion” in relation to a cell population refers to the proportion of stem cells maintaining the undifferentiated state is increased in the cell population. By observing cells, it can be determined whether or not promotion of expansion occurs.

Bmi-1 or a variant or fragment thereof and/or a Bmi-1 regulating agent for use in the present invention may be exogenous or endogenous. Preferably, the agent is exogenous. In the present invention, an exogenous Bmi-1 or its equivalent is provided to a cell, thereby making it possible to regulate (particularly, promote) the expansion of the cell. This effect could not be conventionally predicted. Bmi-1 or a variant or fragment thereof and/or a Bmi-1 regulating agent of the present invention may be endogenous. In this case, an endogenous Bmi-1 may be supplemented with an exogenous agent to enhance the effect thereof.

Bmi-1 or a variant or fragment thereof and/or a Bmi-1 regulating agent for use in the present invention my be in the form of a nucleic acid or a protein, or other forms (e.g., a small molecule, a lipid molecule, a sugar, or a complex thereof).

According to one embodiment of the present invention, Bmi-1 or a variant or fragment thereof and/or a Bmi-1 regulating agent, which is used in the form of a protein, may be:
(a) a polypeptide encoded by a nucleic acid sequence as set forth in SEQ ID NO:1 or 3 (Accession No. L13689 or M64279, respectively) or a fragment thereof;
(b) a polypeptide having an amino acid sequence as set forth in SEQ ID NO:2 or 4 or a fragment thereof;
(c) a variant polypeptide having an amino acid sequence as set forth in SEQ ID NO:2 or 4 having at least one amino acid mutation selected from the group consisting of substitutions, additions, and deletions, the variant polypeptide having a biological activity; or
(d) a polypeptide having at least 70% amino acid sequence homology to any one of polypeptides (a) to (c) and having biological activity.

In a preferred embodiment, the number of substitutions, additions, and deletions in (c) may be limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. A smaller number of substitutions, additions, and deletions is preferable. However, the number of substitutions, additions, and deletions may be large as long as the biological activity, which is preferably similar or substantially the same as the activity of Bmi-1, may be retained.

In another preferred embodiment, the biological activity of the variant polypeptide of (d) includes, but is not limited to, an interaction with an antibody specific to a polypeptide having an amino acid sequence as set forth in SEQ ID NO:2 or a fragment thereof, an interaction with a Bmi-1 polypeptide, and the like.

In a preferred embodiment, the homology to any one of the polypeptides of (a) to (c) may be at least about 80%, more preferably at least about 90%, even more preferably at least about 98%, and most preferably at least about 99%.

The polypeptide of the present invention typically has a sequence length of at least three-contiguous amino acids. The length of the polypeptide of the present invention may be as short as possible as long as the peptide is suitable for an application of interest. However, preferably, a longer sequence may be employed. Therefore, preferably, the polypeptide of the present invention may have a length of at least 4 amino acids, more preferably at least 5 amino acids, at least 6 amino acids, at least 7 amino acids, at least 8 amino acids, at least 9 amino acids, or at least 10 amino acids, even more preferably at least 15 amino acids, and still even more preferably at least 20 aminoacids. These lower limits of the amino acid length may be present between the above-specified numbers (e.g., 11, 12, 13, 14, 16, and the like) or above the above-specified numbers (e.g., 21, 22, . . . , 30, and the like). The polypeptide of the present invention may be identical to the full-length sequence as set forth in SEQ ID NO:2 or longer as long as the polypeptide can interact with a certain element.

In one embodiment, a Bmi-1 polypeptide or a fragment or variant thereof comprises the full-length amino acid sequence as set forth in SEQ ID NO:2. More preferably, Bmi-1 or a fragment or variant thereof may advantageously consist of the full-length amino acid sequence as set forth in SEQ ID NO:2.

In one embodiment of the present invention, Bmi-1 or a variant or fragment thereof and/or a Bmi-1 regulating agent, which is used in the form of a nucleic acid, may be:
(a) a polynucleotide having a base sequence as set forth in SEQ ID NO:1 (Accession No. L13689) or a fragment thereof;
(b) a polynucleotide encoding an amino acid sequence as set forth in SEQ ID NO:2 or a fragment thereof;
(c) a polynucleotide encoding a variant polypeptide having an amino acid sequence as set forth in SEQ ID NO:2 having at least one amino acid mutation consisting of substitutions, additions, and deletions, and having biological activity;
(d) a polynucleotide encoding a polypeptide hybridizable to any one of the polynucleotides of (a) to (c) under stringent conditions; or
(e) a polynucleotide encoding a polypeptide having a base sequence having at least 70% identity to any one of the polynucleotides of (a) to (c) or a complementary sequence thereof and having biological activity.

In a preferred embodiment, the number of substitutions, additions, and deletions in (c) may be limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. A smaller number of substitutions, additions, and deletions is preferable. However, the number of substitutions, additions, and deletions may be large as long as the biological activity, which is preferably similar or substantially the same as the activity of Bmi-1, may be retained.

In another preferred embodiment, the biological activity of the variant polypeptide includes, but is not limited to, an interaction with an antibody specific to a polypeptide having an amino acid sequence as set forth in SEQ ID NO:2 or a fragment thereof, formation of a complex with Mel-18, formation of a complex with Mph1/Rae-28, and the like. These activities can be measured by an immunological assay, quantiatation of phosphorylation, or the like.

In a preferred embodiment, the identity to any one of the polynucleotides of (a) to (c) or a complementary sequence thereof may be at least about 80%, more preferably at least about 90%, even more preferably at least about 98%, and still even more preferably at least about 99%.

In a preferred embodiment, a nucleic acid molecule encoding Bmi-1 of the present invention or a fragment and variant thereof may have a length of at least 8 continuous nucleotides. The appropriate nucleotide length of the nucleic acid molecule of the present invention may vary depending on the application purpose of the present invention. More preferably, the nucleic acid molecule of the present invention may have a length of at least 10 contiguous nucleotides, even more preferably at least 15 contiguous nucleotides, and still even more preferably at least 20 contiguous nucleotides. These lower limits of the aminoacid length may be present between the above-specified numbers (e.g., 11, 12, 13, 14, 16, and the like) or above the above-specified numbers (e.g., 21, 22, . . . , 30, and the like). The upper limit of the length of the nucleic acid molecule of the present invention may be the full length sequence of SEQ ID NO:1 (Accession No. L13689) or more as long as the nucleic acid molecule can be used for an application of interest (e.g., antisense, RNAi, a marker, a primer, a probe, an interaction with a prescribed element). Alternatively, the nucleic acid molecule of the present invention, which is used as a primer, typically has a length of at least about 8 nucleotides, and more preferably about 10 nucleotides. The nucleic acid molecule of the present invention, which is used as a probe, typically has a length of at least about 15 nucleotides, and more preferably about 17 nucleotides.

In one embodiment, a nucleic acid molecule encoding Bmi-1, or a fragment or variant thereof, comprises the full length of the nucleic acid sequence as set forth in SEQ ID NO:1 (Accession No. L13689). More preferably, a nucleic acid molecule encoding Bmi-1, or a fragment or variant thereof, consists of the full length of the nucleic acid sequence as set forth in SEQ ID NO:1 (Accession No. L13689).

In a preferred embodiment, the present invention may comprise an additional cellularly phisiologically active substance. Examples of such a cellularly phisiologically active substance include, but are not limited to, interleukins, chemokines, hematopoietic factors such as colony stimulating factors, a tumor necrosis factor, interferons, a platelet-derived growth factor (PDGF), an epidermal growth factor (EGF), a fibroblast growth factor (FGF), a hepatocyte growth factor (HGF), an endothelial cell growth factor (VEGF), cardiotrophin, and the like, which have proliferative activity. In a particular embodiment, such a cellularly phisiologically active substance used is selected from the group consisting of SCF, TPO, and Flt-3L. This is because SCF, TPO, and Flt-3L have been reported to have an effect of maintaining undifferentiation. In this case, all of SCF, TPO, and Flt-3L may be used.

Cellularly phisiologically active substances, such as cytokines, growth factors, and the like, typically have redundancy in function, Accordingly, reference herein to a particular cytokine or growth factor by one name or function also includes any other names or functions by which the factor is known to those of skill in the art, as long as the factor has the activity of a cellularly phisiologically active substance for use in the present invention. Cytokines or growth factors can be used in a preferred embodiment of the present invention as long as they have preferable activity as described herein.

In the present invention, any cellularly phisiologically active substance may be used. In a preferred embodiment of the present invention, as a cellularly phisiologically active substance, a cytokine or growth factor having hematopoietic activity, colony stimulating activity, or cell proliferative activity Examples of a cytokine having hematopoietic activity or colony stimulating activity include a leukemia inhibitory factor (LIF), a granulocyte macrophage colony stimulating factor (GM-CSF), a macrophage colony stimulating factor (M-CSF), a granulocyte colony stimulating factor (G-CSF), a multi-CSF (IL-3), erythropoietin (EPO), c-kit ligand (SCF), and the like. Examples of a growth factor having cell proliferative activity include a platelet-derived growth factor (PDGF), an epidermal growth factor (EGF), a fibroblast growth factor (FGF), a hepatocyte growth factor (HGF), an endothelial cell growth factor (VEGF), an insulin-like growth factor (IGF), and the like. In a preferred embodiment of the present invention, a cellularly phisiologically active substance (e.g., a cytokine or a growth factor) having cell proliferative activity may be used. In a preferred embodiment, such a cellularly phisiologically active substance includes SCF, TPO, and Flt-3L.

Cellularly phisiologically active substances, such as cytokines and growth factors, can also be divided into categories in accordance with their receptors (e.g., cytokine receptors, etc.). Cytokine receptors are divided into non-kinase type and kinase type. Examples of the non-kinase type include G-protein binding type receptors, an NGF/TNF receptor family, an IFN receptor family, a cytokine receptor superfamily, and the like. Examples of the kinase type include a growth factor type receptor (tyrosine kinase type, such as c-met (for HGF)), a TGFβ receptor family (serine/threonine kinase type), and the like. In some cases, cellularly phisiologically active substances share a receptor subunit. Therefore, a cytokine or growth factor, which shares a receptor subunit with the above-described preferable cytokines or growth factors, may be a preferable cytokine or growth factor.

Cellularly phisiologically active substances, such as cytokines and growth factors, may also be divided into categories in accordance with homology comparison when they are provided in the form of a protein or a nucleic acid. Therefore, in a preferred embodiment of the present invention, a cellularly phisiologically active substance having homology to a preferable cellularly phisiologically active substance of the present invention may be used. Such a cellularly phisiologically active substance has at least about 30% homology to a control cellularly phisiologically active substance when BLAST is employed to perform comparison with default parameters, preferably about 35%, about 40%, about 45%, about 50%, about 55%, about 60%, about 65%, about 70%, about 75%, about 80%, about 85%, about 90%, about 95%, and about 99% homology.

In a preferred embodiment, Bmi-1 for use in the present invention may be advantageously complexed with Mel-18. This is because such a complex was demonstrated to promote the expansion of hematopoietic cells according to the present invention. More preferably, Bmi-1 is advantageously complexed with Mph1/Rae-28. This is because such a complex was demonstrated to promote the expansion of hematopoietic cells according to the present invention.

Bmi-1 or a fragment or variant thereof and/or a Bmi-1 regulating agent for use in the present invention may be consistently active or transiently active. Temporary activity or consistent activity depends on the purpose of the application.

In another preferred embodiment, the Bmi-1 regulating agent of the present invention includes Bmi-1 activating agent. Such an activating agent can be obtained by screening substance libraries using techniques well known in the art. Examples of such a Bmi-1 activating agent include, but are not limited to, a molecule capable of controlling the phosphorylated state of Bmi-1, a molecule capable of controlling the expression of Bmi-1 at the transcription level, and the like.

In another preferred embodiment, Bmi-1 or a fragment or variant thereof and/or a Bmi-1 regulating agent of the present invention may be advantageously in the form of a protein or a complexed protein. Alternatively, Bmi-1 or a fragment or variant thereof and/or a Bmi-1 regulating agent of the present invention may be in the form of a nucleic acid.

In the case of nucleic acid form, such a nucleic acid may be contained in a vector. Such a vector may be a retrovirus vector.

In the present invention, it is intended to provide a drug by computer modeling based on the disclosure of the present invention.

In another embodiment of the present invention, a compound is also provided, which is used as a tool for screening for an agent effective as an active ingredient (e.g., a polypeptide or a nucleic acid) of the present invention and which is obtained by a quantitative structure activity relationship (QSAR) modeling technique using a computer. Here, the computer technique includes several substrate templates prepared by a computer, pharmacophores, homology models of an active portion of the present invention, and the like. In general, a method for modeling a typical characteristic group of a substance, which interacts with another substance, based on data obtained in vitro includes a recent CATALYST™ pharmacophore method (Ekins et al., Pharmacogenetics, 9:477 to 489, 1999; Ekins et al., J. Pharmacol. & Exp. Ther., 288:21 to 29, 1999; Ekins et al., J. Pharmacol. & Exp. Ther., 290:429 to 438, 1999; Ekins et al., J. Pharmacol. & Exp. Ther., 291:424 to 433, 1999), a comparative molecular field analysis (CoMFA) (Jones et al., Drug Metabolism & Disposition, 24:1 to 6, 1996), and the like. In the present invention, computer modeling may be performed using molecule modeling software (e.g., CATALYST™ Version 4 (Molecular Simulations, Inc., San Diego, Calif.), etc.).

The fitting of a compound with respect to an active site can be performed using any of various computer modeling techniques known in the art. Visual inspection and manual operation of a compound with respect to an active site can be performed using a program, such as QUANTA (Molecular Simulations, Burlington, Mass., 1992), SYBYL (Molecular Modeling Software, Tripos Associates, Inc., St. Louis, Mo., 1992), AMBER (Weiner et al., J. Am. Chem. Soc., 106:765-784, 1984), CHARMM (Brooks et al., J. Comp. Chem., 4:187 to 217, 1983), or the like. In addition, energy minimization can be performed using a standard force field, such as CHARMM, AMBER, or the like. Examples of other specialized computer modeling methods include GRID (Goodford et al., J. Med. Chem., 28:849 to 857, 1985), MCSS (Miranker and Karplus, Function and Genetics, 11:29 to 34, 1991), AUTODOCK (Goodsell and Olsen, Proteins: Structure, Function and Genetics, 8:195 to 202, 1990), DOCK (Kuntz et al., J. Mol. Biol., 161:269 to 288, 1992), and the like. Further, structural compounds can be newly constructed using an empty active site, an active site of a known small molecule compound with a computer program, such as LUDI (Bohm, J. Comp. Aid. Molec. Design, 6:61 to 78, 1992), LEGEND (Nishibata and Itai, Tetrahedron, 47:8985, 1991), LeapFrog (Tripos Associates, St. Louis, Mo.), or the like. The above-described modeling methods are commonly used in the art. Compounds encompassed by the present invention can be appropriately designed by those skilled in the art based on the disclosure of the present specification.

In another aspect of the present invention, a method for treatment or prophylaxis of hematopoiesis-related diseases, disorders, or abnormalities is provided. The method comprises the step of administering Bmi-1 or a variant or fragment thereof and/or a Bmi-1 regulating agent in an amount sufficient for treatment or prophylaxis to a subject requiring such regulation.

In another aspect of the present invention, a pharmaceutical composition for treatment or prophylaxis of hematopoiesis-related diseases, disorders, or abnormalities is provided, which comprises Bmi-1 or a variant or fragment thereof and/or a Bmi-1 regulating agent in an amount sufficient for the treatment or prophylaxis.

The present invention targets any “diseases” requiring a large volume of stem cells, or cells, tissues, and organs differentiated therefrom. The present invention may be intended to treat diseases or disorders related to differentiated cells, tissues, or organs which can be developed as a result of the differentiation or expansion of a stem cell of the present invention.

In one embodiment, the present invention may target hematopoietic and circulatory (blood cells, etc.) diseases or disorders Examples of the diseases or disorders include, but are not limited to, anemia (e.g., aplastic anemia (particularly, severe aplastic anemia), renal anemia, cancerous anemia, secondary anemia, refractory anemia, etc.), cancer or tumors (e.g., leukemia); and after chemotherapy therefor, hematopoietic failure, thrombocytopenia, acute myelocytic leukemia (particularly, a first remission (high-risk group), a second remission and thereafter), acute lymphocytic leukemia (particularly, a first remission, a second remission and thereafter), chronic myelocytic leukemia (particularly, chronic period, transmigration period), malignant lymphoma (particularly, a first remission (high-risk group), a second remission and thereafter), multiple myeloma (particularly, an early period after the onset), and the like. The present invention also targets heart failure, stenocardia, cardiac infarction, arrhythmia, valvular heart diseases, myocardial/pericardial diseases, congenital heart diseases (e.g., atrial septal defect, ventricular septal defect, arterial duct patency, tetralogy of Fallot), arterial diseases (e.g., arterial sclerosis, aneurysm, etc.), venous diseases (e.g., phlebeurysm, etc.), and lymph vessel diseases (e.g., lymphatic edema). With the stem cell expanding agent of the present invention, the above-described diseases could be treated while avoiding conventional side effects of transplantation therapy of naturally-occurring stem cells or differentiation cells (particularly, caused by foreign matter or heterogenous cells, (e.g., infection, graft-versus-host diseases, etc.)). This effect could be efficiently achieved only after an agent capable of arbitrarily expanding stem cells was utilized, and can be said to be impossible or difficult to achieve by conventional techniques.

A specific or preferred embodiment of the above-described method and composition is similar to that of the above-described method and composition for regulating the expansion of the above-described hematopoietic stem cell. The amount of a compound effective for diagnosis, prophylaxis, treatment, or prognosis as used herein can be easily determined by those skilled in the art with reference to various parameters, such as the purpose of use, target disease (type, severity, and the like), the patient's age, weight, sex, and case history, the form or type of the cells, and the like (see, “Hikkei Ketsueki Naika Shinryo Handobukku [Companion Handbook of Blood-Internal Medicine Practice], Hideaki Mizoguchi, 1999, Nankodo).

When the present invention is used as a medicament, an additional pharmaceutical agent, such as a stem cell factor (SCF), thrombopoietin (TPO), or the like, can be preferably contained in the medicament.

In another aspect of the present invention, a kit for regulating the expansion of a hematopoietic stem cell is provided. The kit comprises (A) a composition comprising Bmi-1 or a variant or fragment thereof and/or a Bmi-1 regulating agent in an amount sufficient for regulation of the expansion; and (B) instructions setting forth a method of providing the composition to the hematopoietic stem cell and culturing the hematopoietic stem cell. Associated with constituent element(s) (e.g., container(s), etc.) of such a kit can be a notice in the form prescribed by a governmental agency regulating the manufacture, use or sale of pharmaceuticals or biological products, reflecting approval by the agency of the manufacture, use or sale of the product for human administration.

In another aspect of the present invention, a method for producing a cell differentiated from a hematopoietic stem cell line is provided. The method comprises the steps of (A) providing a hematopoietic stem cell or a primordial cell; (B) providing, to the hematopoietic stem cell or primordial cell, Bmi-1 or a variant or fragment thereof and/or a Bmi-1 regulating agent in an amount sufficient for regulation of the expansion; and (C) culturing the hematopoietic stem cell for a time sufficient for regulation of the expansion. Here, the hematopoietic stem cell or the primordial cell may be a primary culture cell or a cultured cell.

In another aspect, the present invention relates to a cell, a tissue, an organ, or an organism produced by promotion of the expansion of a hematopoietic stem cell of the present invention, and a medicament containing them.

Therefore, in another aspect of the present invention, a method for treatment or prophylaxis of a disease or disorder requiring a hematopoietic stem cell or a cell differentiated therefrom is provided. The method comprises (A) administering a cell obtained by a method of the present invention to a subject requiring treatment or prophylaxis of the disease or disorder.

The present invention also relates to use of an agent of the present invention (e.g., a polypeptide, etc.) for the purpose of the present invention (e.g., therapy, diagnosis, prophylaxis, treatment, prognosis, and the like of hematopoiesis-related diseases, disorders, and abnormalities) or use of an agent of the present invention for manufacture of a pharmaceutical composition. Detailed embodiments of the use are similar to those as described above and can be appropriately applied by those skilled in the art.

In another aspect of the present invention, a method for screening for an agent for regulating the expansion of a hematopoietic stem cell is provided. The method comprises. (A) providing candidate substances for the agent; (B) exposing a cell containing Bmi-1 to the substances; and (C) determining whether or not the Bmi-1 is regulated, wherein if the Bmi-1 is regulated, the substance is then determined to be an agent capable of regulating the expansion of the hematopoietic stem cell. In this case, when the Bmi-1 is upregulated, the substance may be determined to be an agent capable of promoting the expansion of a hematopoietic stem cell. The candidate substances may be provided in a library. The present invention provides an agent for regulating (preferably, promoting) the expansion of a hematopoietic stem cell obtained by the above-described screening method.

Hereinafter, the present invention will be described by way of examples. Examples described below are provided only for illustrative purposes. Accordingly, the scope of the present invention is not limited except as by the appended claims.

### EXAMPLES

The present invention will be described in greater detail by way of examples. The present invention is not limited to the examples below. Animals were treated in accordance with rules defined by The University of Tokyo (Japan).

(Common Experimental Method)

(Mice)

C57BL/6 (B6-Ly5.2) mice were purchased from Charles River Japan, Inc. The mice were congenic at the Ly5 locus (B6 Ly5.1) and were cross-bred and treated in compliance with the spirit of animal protection in the Animal Research Center of The Institute of Medical Science in The University of Tokyo.

(Plasmids)

Mouse Bmi-1, M33, and Mph-1 were obtained from Dr. H. Koseki of Chiba University (Japan), Dr. T. Higashinakagawa of Waseda University (Japan), and Dr. M. van Lohuizen of The Netherlands. Cancer Institute (Netherlands), respectively. The nucleic acid sequences and amino acid sequences of the three genes are set forth in SEQ ID NO:3 (Accession No. M64279) (Bmi-1), SEQ ID NO:15 (Accession No. BC035199) (M33), and SEQ ID NO:11 (Accession No. U63386) (Mph-1). cDNAs of mouse Bmi-1 and Mph-1 were FLAG-tagged at the N-terminus thereof. A series of Bmi-1 mutant cDNAs were FLAG-tagged at the N-terminus thereof, followed by PCR amplification. These cDNAs were designated as follows: RING finger domain deletion (DRF; D18-56; SEQ ID NO:19); HTHTHT domain deletion (DHT; D165-220; SEQ ID NO:21); and proline/serine-rich region deletion (DP/S; D248-324; SEQ ID NO:23). A Hox gene was obtained from Dr. R. K. Humphries of Terry Fox Laboratory (Canada). Deletion mutants were obtained by site-specific mutagenesis techniques well known in the art.

(Production of Retroviruses)

A retrovirus vector GCDsam (pGCDsam) was used. The vector contains an LTR derived from MSCV, and intact splice donor and splice acceptor sequences for production of subgenomic mRNA (Kaneko S. et al., Human Gene Therapy, 12:35-44, 2001). Mouse Bmi-1, M33, Mph-1, and a series of Bmi-1 mutant cDNA were subcloned at an upstream site of an IRES-EGFP construct of pGCDSam. To produce a recombinant retroviral gene, plasmid DNA was transfected to a 293 gp cell. The 293 cell contains gag and pol genes but lacks an envelope gene. Here, transfection was performed by coprecipitation of an expression plasmid VSV-G with CaPO₄. The supernatant containing the transfected cells was centrifuged at 6,000×g for 16 hours. The resultant pellet was resuspended in S clone medium (1/200 of the early volume of the supernatant). The titer of the vector was determined by infecting Jurkat cells (human T cell line) with the vector. In this case, the Jurkat cells were infected with serial dilutions of a virus stock, and among the Jurkat cells, GFP-positive cells were subjected to FACS analysis (FACS Callibur, Beckton Dickinson).

(Isolation of Mouse Hematopoietic Stem Cell)

Mouse hematopoietic stem cells (CD34⁻c-Kit⁺Sca-1⁺Lineage-marker-cell) were isolated from bone marrow cells of 2-month-old B6-Ly5.1 mice (Osawa M., Hanada K., Hamada H., Nakauchi H., Science, 1996 Jul. 12; 273(5272):242-5). Briefly, low-density cells were isolated using Lymphoprep (1.086 g/ml; Nycomed, Oslo, Norway). The isolated cells were stained using an antibody cocktail comprising monoclonal antibodies, i.e., a biotinylated anti-Gr-1 (RB6-8C5), Mac-1 (Ml/70), B220 (RA3-6B2), CD4 (GK1.5), CDB (53-6.7), and Ter-119 (PharMingen, San Diego, Calif.). The cells were further stained with fluorescein isothiocyanate (FITC)-labeled anti-CD34 antibody (49E9, PharMingen), phycoerythrin (PE)-labeled anti-Sca-1 antibody (PharMingen), and allophycocyanin (APC)-labeled anti-c-Kit antibody (ACK-2; PharMingen). The biotinylated antibodies were subjected to color development using streptavidin-Texas. Red (Life Technologies). Four color analysis and sorting were performed using FACS Vatage™ (Becton Dickinson, San Jose, Calif.). Dead cells were eliminated by propidium iodide staining. CD34−KSL cells were placed in 96-well microtiter plates coated with recombinant fibronectin fragments (Takara Shuzo, Otsu, Japan) at 150 cell per well.

(Expression of Bmi-1 in Hematopoietic Cell)

Expression of Bmi-1 was observed in various cells. The expression was observed by RT-PCR. Bone marrow cells, spleen cells and thymocytes were used.

Expression of Bmi-1 in hematopoietic cells is shown in FIG. 1.

(Transduction of CD34−KSL Cell)

CD34−KSL cells were incubated in S-Clone SF-03 (Sanko Junyaku, Tokyo, Japan) supplemented with 1% fetal bovine serum (FBS), 50 ng/ml stem cell factor (SCF), 100 ng/ml thrombopoietin (TPO (Peprotech, Rocky Hill, N.J.)) for 24 hours. Thereafter, transduction was performed using a retrovirus in the presence of protamine sulfate (5 g/mL; Sigma, St. Louis, Mo.) at a multiplicity of infection (MOI) of 600. After transduction, the cells were further incubated and were subjected to an in vitro colony assay and competitive bone marrow reconstitution activity analysis at indicated time points.

(Colony Assay and In Vitro Liquid Culture)

The CD34−KSL cells were transduced with the indicated retrovirus and were cultured in a methylcellulose medium (Stem Cell Technologies, Vancouver, BC) containing 20 ng/ml SCF, IL-3, and 50 ng/ml TPO, and 2 units/ml EPO (these were obtained from Peprotech). Culture dishes were incubated in 5% CO₂ atmosphere at 37° C. The number of colonies was counted on Day 10.

(Study on Competitive Bone Marrow Reconstitution Ability)

Competitive bone marrow reconstitution ability was studied using an Ly5 system as described in Osawa M., Hanada K., Hamada H., Nakauchi H., science, 1996 Jul 12; 273(5272):242-5). Briefly, transduced Ly5 mouse-derived hematopoietic stem cells were mixed with 2×10⁵ bone marrow cells (B6-Ly5.2). The mixed cells were transplanted in B6-Ly5.2 mice subjected to radiation at a dose of 9.5 Gy. Four weeks and 12 weeks after transplantation, the recipient's peripheral blood cells were stained with biotinylated anti-Ly5.1 (A20) and FITC-labeled antibody Ly5.2 (Osawa M., Hanada K., Hamada H., Nakauchi H., Science, 1996 Jul. 12; 273(5272):242-5). These cells were simultaneously stained with a PE-labeled anti-B220 antibody or a PE-labeled anti-Mac-1 antibody and a FE-labeled anti-Gr-1 antibody or a combination of a PE-labeled anti-CD4 antibody and a PE-labeled anti-CD8 antibody. The biotinylated antibodies were subjected to color development using streptavidin APC (PharMingen). The cells were analyzed on FACS Vantage™. The percentage of chimerism was calculated by (percentage of Ly5.1 cell)×100/(percentage of Ly5.1 cell+percentage of Ly5.2 cells). The chimerism percentage is 1% or more with respect to cells in the peripheral blood and reconstitution of myelocytes and lymphocytes by Ly5.1 donor cells is confirmed, it should be regarded that multiple lines of blood cells are reconstituted (positive mouse).

### Example 1

### Promotion of Expansion by Expression of Exogenous Bmi-1

To confirm promotion of expansion by expression of exogenous bmi-1 using the above-described method, Bmi-1 was introduced into a mouse CD34−KSL hematopoietic stem cell using a retrovirus. Introduction was confirmed by detecting the expression of a genetic product in the cell.

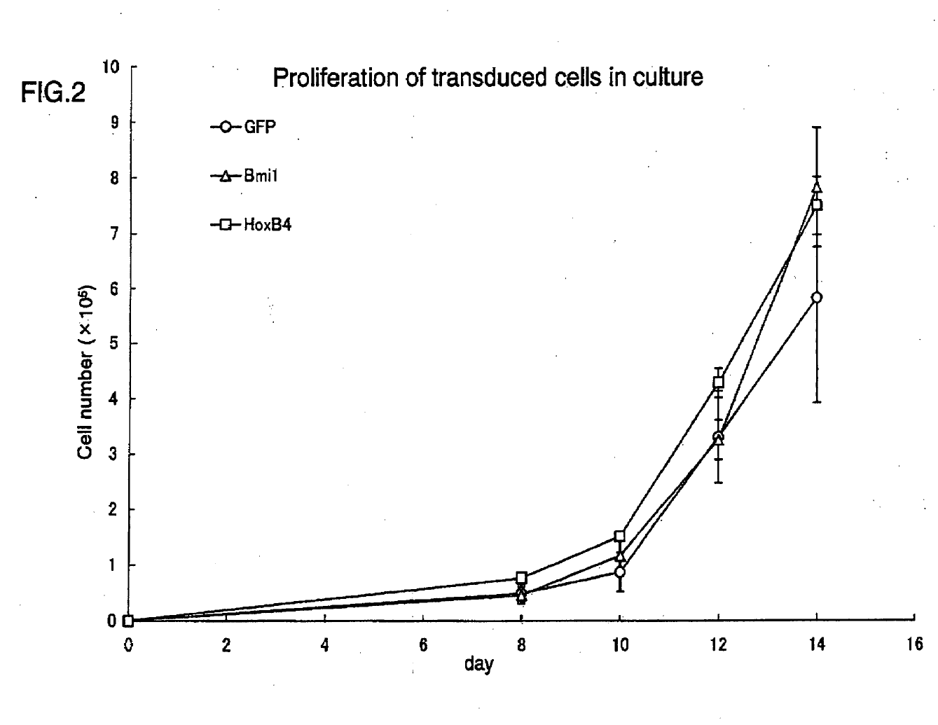

The present inventors transduced the cell with a retrovirus vector GCsam-Bmi-1-IRES-EGFP. Expression of Bmi-1 and an enhanced green fluorescent protein (EGFP) was performed using a single dicistronic message. After transduction, the transduction efficiency was evaluated using a fluorescent inverted microscope. The present inventors achieved an efficiency of about 80% in all experiments. The cell was subjected to an in vitro colony assay on Day 7 and Day 14 (5 days and 12 days after transduction). The Bmi-1-transduced cell culture contained a significantly larger amount of highly proliferative potential colony forming cells (HPP-CFC; colony size >1 mm) than that of a control cell transduced with HoxB4 (control) and that of a non-transduced cell. The results are shown in FIG. 2. The homeobox gene is a gene which is known to control the expansion of a hematopoietic stem cell and a primordial cell thereof in vitro.

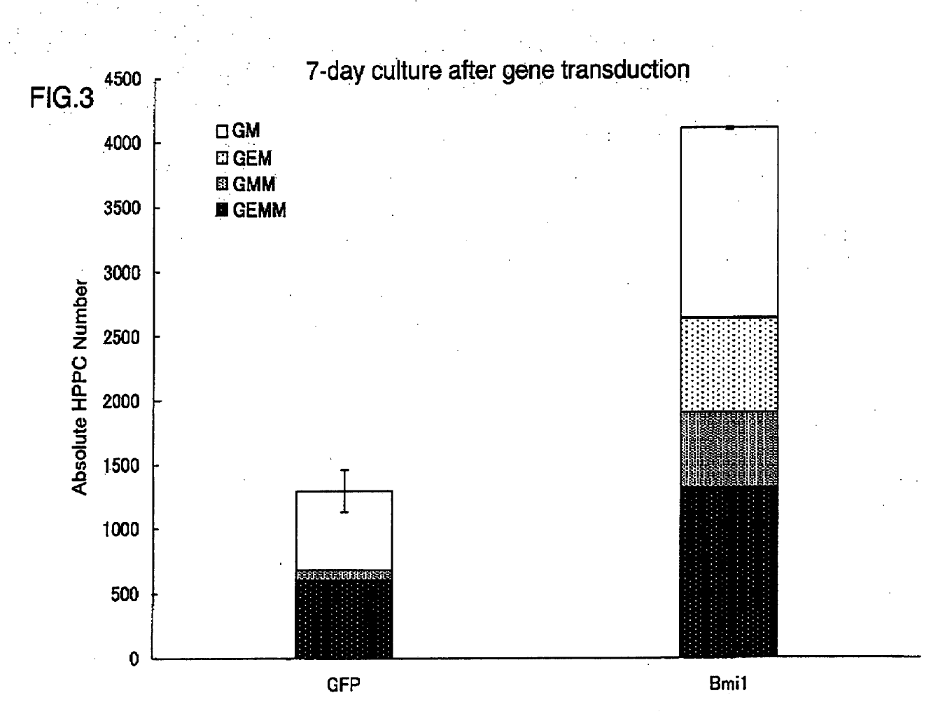

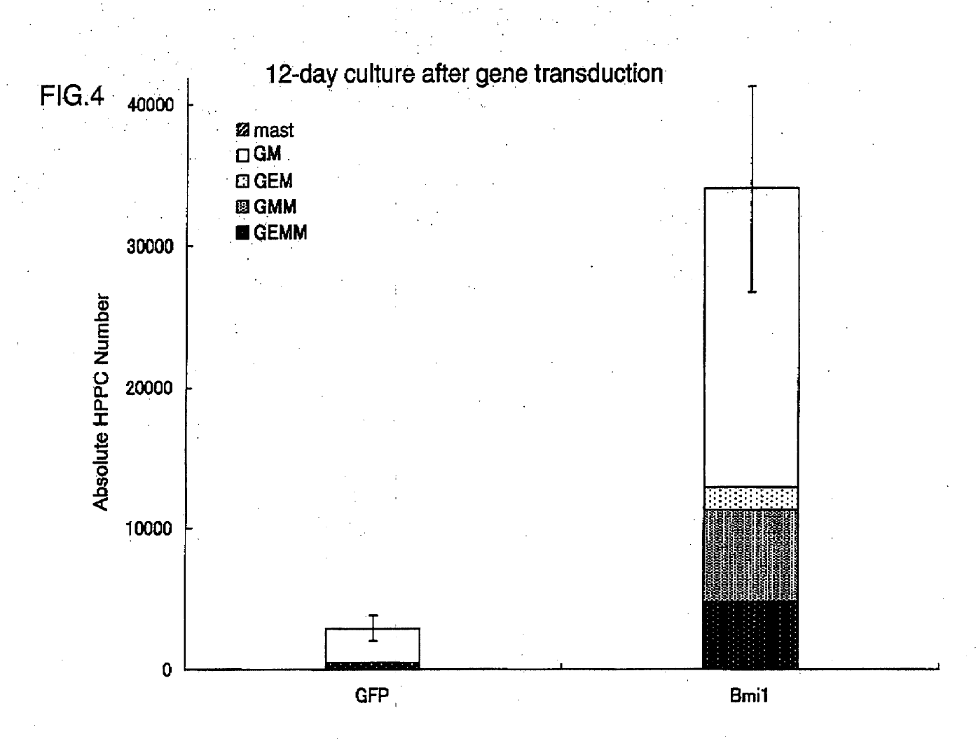

Importantly, the expression of Bmi-1 promoted expansion of pluripotent hematopoietic stem cells/precursor cells and colony forming unit-granulocyte/erythrocyte/macrophage/megakaryocyte (CFU-GEMM) (cultured in the first 7 days; see FIG. 3). On Day 14 of culture, the Bmi-1 transduced cell still had a significantly larger number of HPP-CFCs (absolute HPP number) than that of the control, however, the expansion ability was slightly limited compared with the data on Day 7 (see FIG. 4). The increase in HPP-CFC was responsible for an increase in the total number of cells (FIG. 1). The data show that the expression of Bmi-1 in the hematopoietic stem cell was more efficient than HoxB4 with respect to pluripotent hematopoietic stem cells/precursor cells.

Thus, although substantially no difference was found in the number of cells in liquid culture, expansion of undifferentiated cells (CFU-GEMM, CFU-GEM, and CFU-GMM) were confirmed in colony assays.

Therefore, the Bmi-1 of the present invention exhibited an ability to promote the expansion of a hematopoietic stem cell more than conventional substances.

### Example 2

### Starting with 100 Stem Cells

Next, another in vitro experiment was performed. In Example 2, the effect of the present invention was confirmed by conducting a experiment commencing with the use of 100 stem cells.

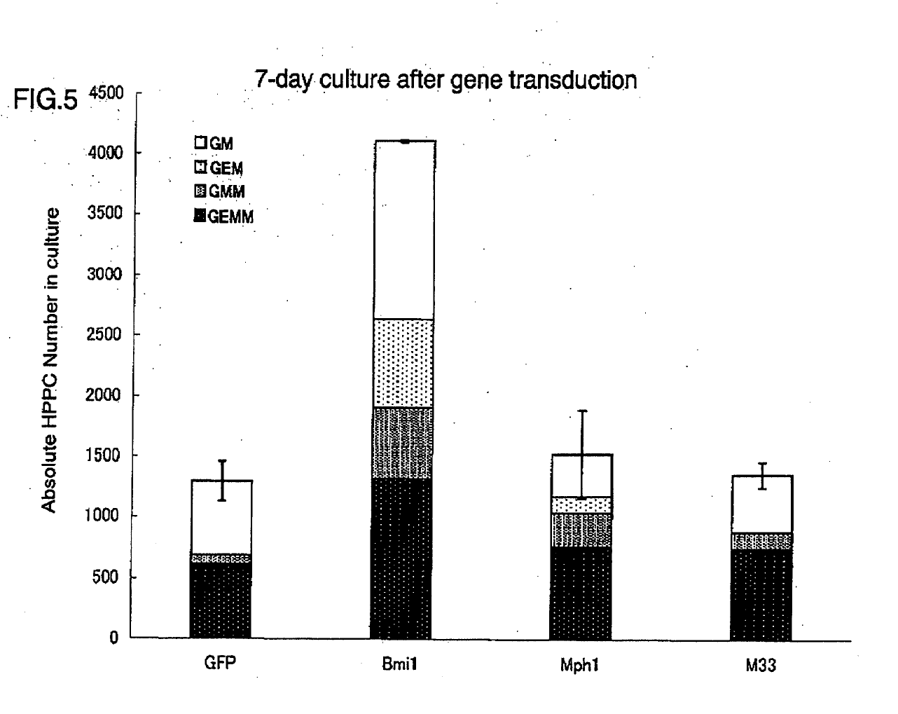

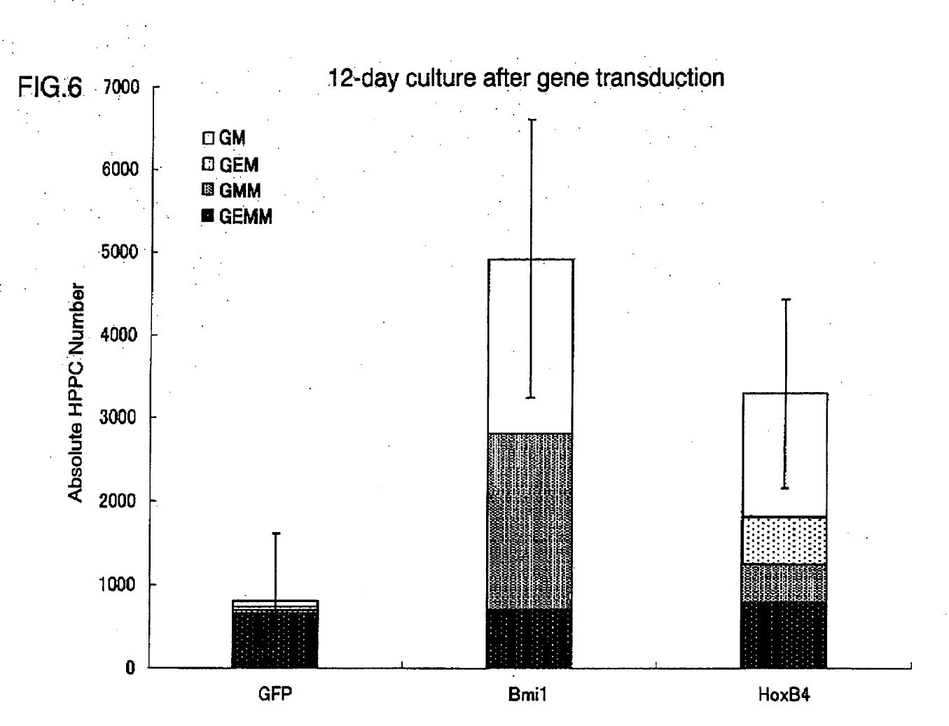

Expression of exogenous Bmi-1 was performed in accordance with a protocol described in Example 1. In Example 2, Bmi-1 was used as an agent of the present invention, a GFP was used singly as a negative-control, and a product of expression of HoxB4 was used as another control. In Example 2, high proliferative potential cells were counted on Day 7 and Day 14 of culture, The results are shown in FIGS. 5 and 6.

As shown in FIGS. 5 and 6, among stem cells in which the Bmi-1 of the present invention was expressed, the number of cells of lines GM and GMM were significantly increased. The increase was significantly higher than that of the HoxB4 cell. Therefore, the Bmi-1 of the present invention exhibited a higher level of hematopoietic stem cell expansion promoting activity than that of conventional substances. The effect appeared to a significant level as early as Day 7. The effect of Bmi-1 was significantly higher than that of the Hox4B (control) molecule.

### Example 3

### Transplantation to Animal Model

Next, to confirm the expansion promoting effect of the Bmi-1 of the present invention, stem cells were transplanted into an animal model, and thereafter, the expansion was observed.

Hereinafter, the experimental protocol is briefly described. Hematopoietic stem cells were obtained and cultured for one day as described in Example 1. Thereafter, as described in Example 1, Bmi-1 was forceably expressed by infection with a retrovirus. After expression, the cells were cultured for 7 to 10 days. After culture, the cells were transplanted into irradiated mice. The mice were C57BL/6 (B6-Ly5.2) mice purchased from Charles River Japan, Inc. For the mice, CD45.1 was a specific marker. After irradiation, 2×10⁵ bone marrow competitive cells (B6-Ly5.2) were transplanted together with the cultured cells. For the bone marrow cells, CD45.2 was a specific marker. Four and eight weeks after, all peripheral blood nuclear cells were analyzed. For the analysis, GFP, Gr-1, Mac1, CD4, CD8, B220, and the like were employed in addition to CD45.1. The detailed protocol is described in the section “Common Experimental Method”. The results are shown in Tables 1 and 2.

TABLE 1
(4 weeks)

| | | | | GRl | | |
|---|---|---|---|---|---|---|
| | | CD45.1 & | GFP + | & | CD (4) & | |
| Sample | CD45.1 | GFP | (%) | Macl | 8 | B220 |
| GFP SF03 1% | 23.4 | 21.7 | 92.7 | 6.6 | 0.5 | 10.9 |
| FBS-1 | | | | | | |
| GFP SF03 1% | 11.4 | 9.7 | 85.0 | 0.6 | 0.0 | 7.0 |
| FBS-2 | | | | | | |
| GFP SF03 1% | 24.7 | 21.5 | 87.0 | 0.7 | 0.6 | 16.7 |
| FBS-3 | | | | | | |
| GFP SF03 1% | 19.4 | 15.8 | 81.4 | 2.6 | 0.1 | 13.3 |
| FBS-4 | | | | | | |
| GFP SF03 1% | 20.2 | 18.8 | 93.1 | 2.5 | 0.0 | 13.7 |
| FBS-5 | | | | | | |
| GFP SF03 1% | 10.6 | 8.3 | 78.7 | 0.3 | 0.3 | 8.1 |
| FBS-6 | | | | | | |
| Bmil SF03 1% | 56.4 | 48.6 | 86.2 | 11.8 | 2.0 | 34.7 |
| FBS-1 | | | | | | |
| Bmil SF03 1% | 49.1 | 40.6 | 82.7 | 10.5 | 1.2 | 27.7 |
| FBS-2 | | | | | | |
| Bmil SF03 1% | 57.3 | 50.0 | 87.3 | 14.2 | 0.0 | 15.9 |
| FBS-3 | | | | | | |
| Bmil SF03 1% | 50.4 | 44.0 | 87.3 | 17.2 | 0.8 | 17.0 |
| FBS-4 | | | | | | |
| Bmil SF03 1% | 48.5 | 43.6 | 89.9 | 11.4 | 2.3 | 25.0 |
| FBS-5 | | | | | | |

TABLE 2
(8 weeks)

| | | CD45.1 & | | Grl & | CD4 & |
|---|---|---|---|---|---|
| Sample | CD45.1 | GFP | GFP (%) | Macl | 8 |
| GFP SF03 1% FBS-1 | 17.8 | 14.7 | 82.5 | 1.75 | 2.87 |
| GFP SF03 1% FBS-2 | 8.4 | 6.33 | 75.3 | 1.66 | 1.93 |
| GFP SF03 1% FBS-3 | 19.5 | 17.2 | 86.6 | 0.9 | 4.08 |
| GFP SF03 1% FBS-4 | 16.1 | 11.4 | 71 | 0.38 | 1.38 |
| GFP SF03 1% FBS-5 | 17.7 | 14.7 | 83.3 | 1.77 | 6.05 |
| GFP SF03 1% FBS-6 | 17.3 | 11.3 | 48.1 | 0.21 | 0.72 |
| Bmil SF03 1% FBS-1 | 35 | 24.5 | 70 | 2.52 | 10.4 |
| Bmil SF03 1% FBS-2 | 52.2 | 40.4 | 77.4 | 4.4 | 11 |
| Bmil SF03 1% FBS-3 | 56.4 | 44.7 | 79.2 | 4.45 | 20.7 |
| Bmil SF03 1% FBS-4 | 61.8 | 48 | 77.7 | 11.3 | 15.6 |
| Bmil SF03 1% FBS-5 | 51.4 | 46.42 | 89.9 | 6.05 | 22.1 |

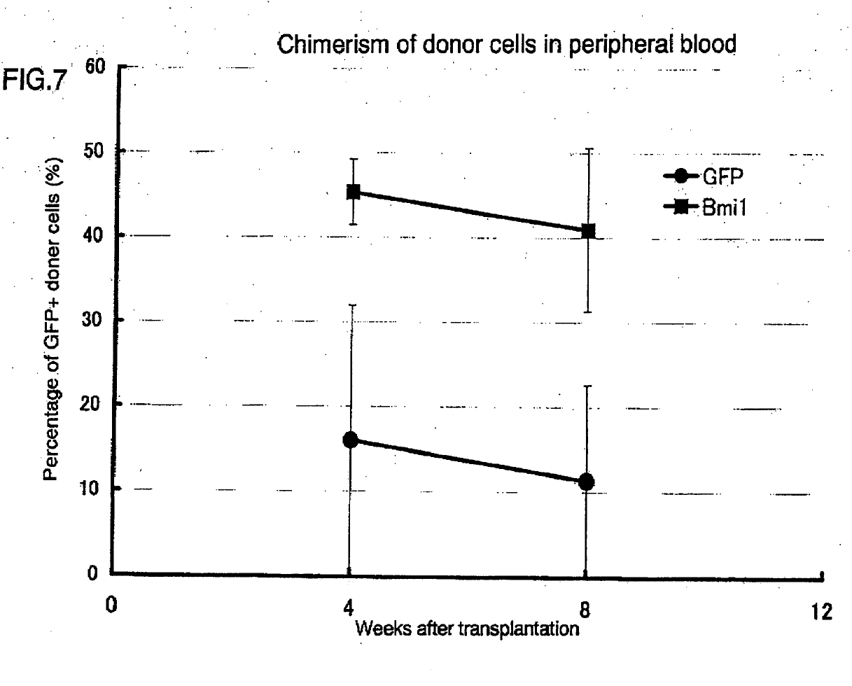

The expression of Bmi-1 is shown in FIG. 7 based on the above-described tables. Thus, the expression of Bmi-1 was observed in donor cells, and therefore, it was confirmed that cell chimerism occurred.

As described above, in Example 3, it was found that the forced expression of Bmi-1 led to promotion of proliferation of hematopoietic stem cells. Therefore, expansion and maintenance of hematopoietic stem cells was shown in the transplantation experiment. Such an effect had not been conventionally known. Therefore, the present invention demonstrated a significant effect.

### Example 4

### Effect of PCG Complex

An experiment similar to that of Example 1 is performed using a PcG complex comprising Bmi-1. In Example 4, instead of Bmi-1, the PcG complex (a complex of Bmi-1, and Mph1/Rae28 or M33) is used. The PcG complex is prepared by preparing the components, mixing and incubating the components.

Hematopoietic stem cells are superinfected with the complex, The effect of superinfection is compared to the effect of single infection. Alternatively, for comparison, hematopoietic cells are superinfected with HoxB4 (the effect of a complex of Bmi-1 and HoxB4 is known).

As a result, it is indicated that even if the Bmi-1 of the present invention is present in a PcG complex, the Bmi-1 exhibits a higher level of hematopoietic stem cell expanding ability than that of conventional substances.

### Example 5

### Use of Cells Obtained by Expansion of Hematopoietic Stem Cells

Bmi-1 is introduced into human hematopoietic stem cells (CD34³⁰ CD38⁻ cells) ex viva, followed by expansion. Thereafter, the stem cells are transplanted into NOD/SCID mice (Charles River Japan). Expansion of bone marrow reconstitution ability due to Bmi-1 is confirmed.

### Example 6

### Screening for Bmi-1-Related Substance

A cell line having agene, in which GFP (SEQ ID NO:17 (Accession No. AJ249646)) is consistently linked to a promoter of a Bmi-1 gene (SEQ ID NO:1 (Accession No. L13689)), is established. The cell is used for screening small molecule compounds for a compound capable of promoting expression of Bmi-1.

The selected compound, which is capable of promoting expression of the Bmi-1 gene, can be used as an agent for expanding a hematopoietic stem cell and a nervous stem cell.

### Example 7

### Screening of Bmi-1-Related Substances

A cell is prepared, in which a green fluorescent protein (GFP; SEQ ID NO:17 (aminoacid sequence: SEQ ID NO:18)) is knocked-in at the locus of p16^{INK4a} (SEQ ID NO:27 (amino acid sequence; SET ID NO:28); chromosome 4; AF044336) or p19^{ARF} (SEQ ID NO:29 (amino acid sequence: SET ID NO:30); chromosome 11; NM_{—}007476). Specifically, the preparation is carried out as follows.

An ES cell in which a GFP gene is knocked in by homologous recombination in-frame at the start codon site of p16^{INK4a} is established. A similar cell strain is established using an ES cell lacking Bmi-1. Using these ES cells, low molecular weight compounds are screened for a compound capable of suppressing expression of p16^{INK4a} only in wild-type ES cells but not in ES cells lacking Bmi-1.

Thus, Bmi-1-related compounds can be screened for.

### Example 8

### Effect of Variant

Next, variants having a RING finger domain deletion (DRF; D18-56; SEQ TD NO:20); a HTHTHT domain deletion (DHT; D165-220; SEQ ID NO: 24); and a proline/serine-rich region deletion (DP/S; D248-324; SEQ ID NO:24), and a sequence having a mutation in Bmi-1 at a desired site of SEQ ID NO:4, are used to determine whether or not a similar effect is obtained.

Such variants are prepared by the above-described method or site-specific mutagenesis. The DNA sequences of the prepared nucleic acid molecules are determined by a sequencer. When a precise sequence is found, the nucleic acid molecule is used to produce variants.

The hematopoietic stem cell proliferating effect of the variant can be observed in accordance with a protocol as described in Examples 1 and 3. Therefore, even in the case of the variants, the hematopoietic stem cell proliferating effect similar to that of Bmi-1 can be found. By modifying the mutant to a further-extent, a Bmi-1 molecule having a more potent effect can be obtained.

### Example 9

### Other Bmi-1-Related Agents

A proliferating effect of Mph-1 and M33 other than Bmi-1, which are involved in signal transduction, was examined. As a control, a system using Hox4B was used. The results are shown in FIGS. 5 and 6. As a result, it was found that mph-1 and M33 exhibited a significantly higher level of hematopoietic stem cell expansion promoting ability than that of the control, but not to the level of the Bmi-1 agent. Therefore, it was demonstrated that PcG-related molecules have an effect similar to that of Bmi-1.

### Example 10

### Effect on Genital System Stem Cells

The present inventors studied an effect of Bmi-1 of the present invention on sperm stem cells (genital system stem cells) as stem cells other than hematopoietic cells.

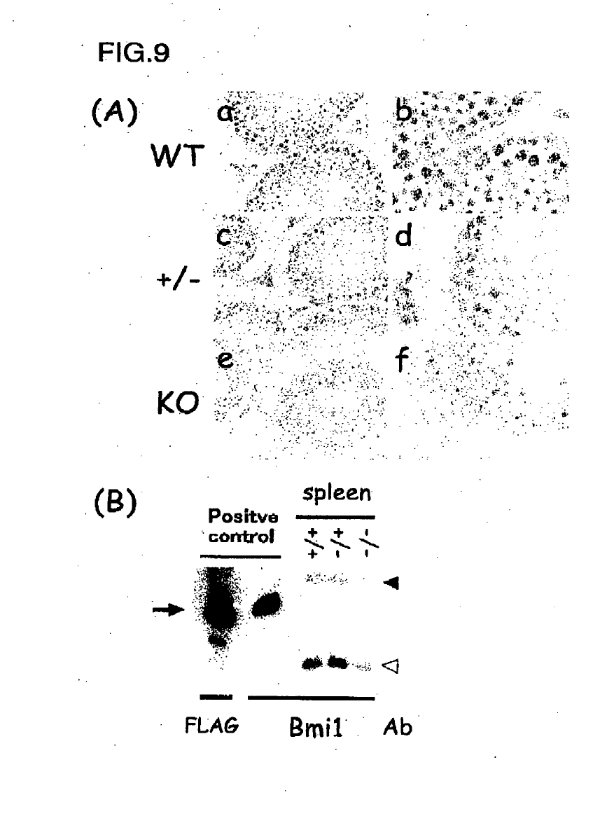

The present inventors confirmed whether or not Bmi-1 is expressed in the testis. Testis sections of 8-week-old wild type mice (WT; a, b), hetero mice (+/−; c, d), and knockout mice (−/−; e, f) were used. The wild type mice were obtained from Charles River Japan. The hetero mice and the knockout mice were obtained by commonly used methods. FIG. 9A shows a photograph of the testis section in which Bmi-1 was immunologically stained. In b, d and f, the magnification was 200×. In a, c and e, the magnification was 400×. Spermatogonium and sperm cells are stained. Knockout mice are slightly stained.

Thereafter, western blotting of Bmi-1 was carried out using the spleen from the same individual from which the testis was removed. The results are shown in FIG. 9B. Lanes 1 and 2 indicate cells in which FLAG-Bmi-1 was expressed as a control. Lanes 2 to 5 indicate anti Bmi-1 antibodies (UBI). Positions indicated by arrows indicate positions of Bmi-1. In lanes 3 to 5, bands indicated by arrow heads indicate Bmi-1. Non-specific bands were observed at positions indicated by open arrow heads. Therefore, the expression of Bmi-1 shown in FIG. 9A is considered to include non-specific staining. In the knockout mice, substantially no stained cell was observed among the spermatogoniums. Therefore, it is considered that Bmi-1 must have been expressed in at least spermatogoniums.

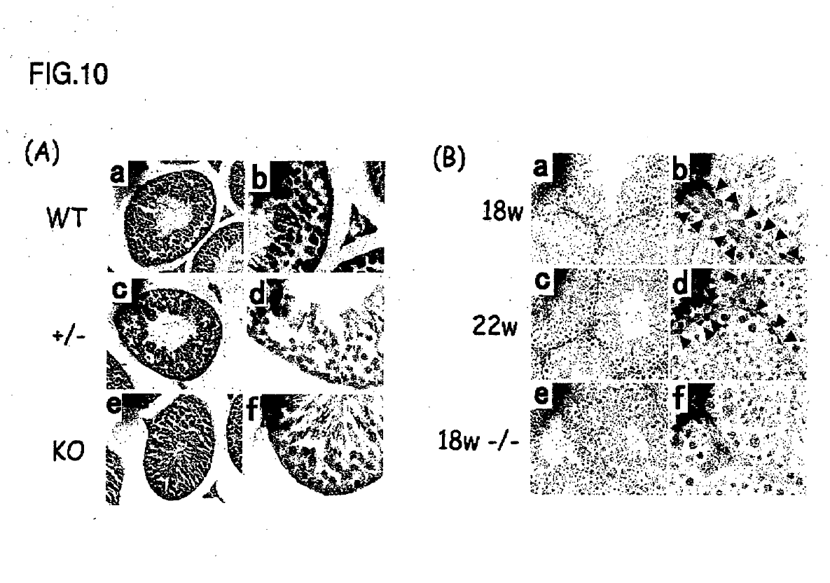

Next, the testes of Bmi-1 knockout mice were subjected to HE staining. HE staining was carried out by a commonly used method as follows. Samples were optionally subjected to deparaffinization (e.g., in pure ethanol), washing with water, and immersion in omni-hematoxylin for 10 min. Thereafter, washing with running water was carried out. Color development was performed with ammonia water for 30 sec. Thereafter, washing with running water was carried out for 5 min. The samples were stained with a 10-fold diluent of eosin hydrochloride for 2 min, followed by dehydration, clearing, and mounting. The testes of 8-week-old wild type mice (WT; a, b), hetero mice (+/−; c, d), and knockout mice (−/−; e, f) were subjected to HE staining. The results are shown in FIG. 10A. In b, d and f, the magnification was 200×. In a, c and e, the magnification was 400×. A number of flat sperm stem cells were observed in the basal lamina. It was found that spermatogenesis was performed and sperm stem cells were present in the Bmi-1 knockout mice. Thereafter, the testes of 18-week-old wild type mice (a, b) and 22-week-old wild type mice (c, d), and 18-week-old Bmi-1 knockout-mice (−/−; e, f) were subjected to HE staining. The results are shown in FIG. 10B. In b, d and f, the magnification was 200×. In a, c and e, the magnification was 400×. In the wild type mice, a number of sperm stem cells were observed in both the 18- and 22-week-old mice as indicated by arrow heads. In the Bmi-1 knockout mice, substantially no cell indicated the specific form of sperm stem cells.

As a result, it was found that Bmi-1 is highly expressed particularly in sperm stem cells. In Bmi-1 knockout mice, as their age proceeded, the number of sperm stem cells was decreased. Therefore, it is considered that Bmi-1 is deeply involved in self-replication of sperm stem cells similar to hematopoietic stem cells.

Based on this finding, it was contemplated that sperm stem cells can be expanded by controlling the function of Bmi-1. To demonstrate this, the following experiment was carried out.

Sperm stem cells are purified from the testes of newborn mice with procedures known in the art. The mice are anesthetized and an incision is made in their abdomen to remove the testes from the scrota. The fat tissue around the epididymis is stitched to the peritoneum using a needle, and the peritoneum and the skin are united to end the operation. Testes are prepared as experimentally undescended testes. The testes are retained for 2 to 3 months before use. Thereafter, the whole testes are removed. The testes are immersed in Hank's solution and a thin film covering the testis is removed. The testis only consisting of seminiferous tubules is transferred into a Falcon tube, followed by incubation in collagenase solution at 32° C. for 15 min while stirring appropriately. Therefter, the collagenase solution is removed and the testis is rinsed with Hank's solution (e.g., twice). Thereafter, 0.25% trypsin/DNase (7 mg/ml) solution (amount ratio of 4:1) is added, followed by incubation for 10 min. In this case, the seminiferous tubules are well raveled, followed by pippetting. Trypsin reactions are neutralized with an equal amount of PBS/1% FBS solution, followed by centrifugation at 1,500 rpm for 5 min. The pellet is suspended in an appropriate amount of PBS/1% FBS. The cells are immunologically stained. 10⁶ cells are present per 100 μl and 1 μg of antibodies are added thereto. This is used in cell sorting. The antibodies are anti-α₆-integrin antibodies labeled with PE and biotinylated anti-αᵥ-integrin antibodies, which are stained with streptavidin. PBS/1% FBS can be used as buffer solution for staining. PI is used to remove dead cells. Preparation of cell sorting is ended. The prepared cells are subjected to cell sorting (FACS Vantage; 488 nm argon laser and 633 nm helium neon laser are used).

A Bmi-1 gene is introduced into the purified sperm stem cells via lentivirus. The cells are transplanted into the testes of recipients. The expansion of the stem cells is examined.

There are various causes for male sterility. Aspermatogenesis is the major cause. Therefore, control of the function of Bmi-1 leads to the solution. It is recognized that control of the function of Bmi-1 is useful for treatment of diseases and abnormalities of genital functions.

### Example 11

### Neural Stem Cells

It is considered that the number of sperm stem cells is gradually decreased in Bmi-1 knockout mice. Therefore, it is considered that the effect of the present invention can be obtained in sperm stem cells.

It is considered that Bmi-1 is also expressed in nerves and is involved in self-replication of neural stem cells. Therefore, the possibility of expansion of neural stem cells is studied. Neural stem cells are purified from mouse ventricular substratums with a procedure known in the art and are cultured in vitro. Purification can be carried out as follows.

Embryonic mice are removed from pregnant mice and are decapitated. Their brains are transferred into PBSG. The corpus striatums or ventricular substratums are excised from the brains with forceps and scissors in the PBSG under a stereoscopic microscope. The tissue obtained is transferred into a 15-ml tube, followed by removal of PBSC with a pipette. 100 μl of MHM culture medium is added per tissue obtained from two mice. The tissue is sectioned into small pieces by pipetting.

MHM culture solution (100 μl) is added and the number of cells is counted. The cells are suspended in MHM culture solution supplemented with a growth factor (20 ng/ml EGF or 20 ng/ml bFGF, or both thereof) at a concentration of 2×10⁵ cells/ml or less, followed by culture at 37° C. in 5% CO₂ for 7 days. The cells are used as neural stem cells.

Alternatively, neurospheres can be isolated from adult mice to prepare neural stem cells. Neurospheres are cell masses and can be cultured in vitro. This system can be analyzed in a manner similar to that for hematopoietic cells.

Retroviruses are used to study and determine whether or not neural stem cells can be expanded by introduction of Bmi-1 or whether or not functional neural cells can be induced from the expanded neural stem cells and can be transplanted into mice. According to the analysis, a limited amount of neural stem cells can be efficiently expanded, so that neural cells for cell treatment can be efficiently supplied.

### Example 12

### The Role of Different Components of the Bmi-1-Containing Complex in HSC

Experimental Procedures

Mice

Bmi-1^{-/-} mice (van der Lugt, N. M., et al., Genes & Dev. 8, 757-769, 1994), Mel-18^{-/-} mice (Akasaka, T., et al., Development 122, 1513-1522, 1996), M33^{-/-} mice (Katoh-Fukui, Y., et al., Nature 393, 688-693, 1998), and p19^{-/-} mice (Kamijo, T., et al., Cell 91, 649-659, 1997) that had been backcrossed at least eight times onto a C57BL/6 (B6-Ly5.2) background were used in this invention. C57BL/6 (B6-Ly5.2) mice were purchased from Charles River Japan, Inc. Mice congenic for the Ly5 locus (B6 Ly5.1) were bred and maintained at the Animal Research Center of the Institute of Medical Science, University of Tokyo.

Purification of Mouse Hematopoietic Stem Cells

Mouse hematopoietic stem cells (CD34−KSL cells) were purified from bone marrow cells of 2-month-old mice. In brief, low-density cells were isolated on Lymphoprep (1.086 g/ml; Nycomed, Oslo, Norway). The cells were stained with an antibody cocktail consisting of biotinylated anti-Gr-1, Mac-1, B220, CD4, CD8, and Ter-119 mAbs (PharMingen, San Diego, Calif.). Lineage-positive cells were depleted with streptavidin-magnetic beads (M-280; Dynal Biotech, Oslo, Norway). The cells were further stained with fluorescein isothiocyanate (FITC)-conjugated anti-CD34, phycoerythrin (PE)-conjugated anti-Sca-1, and allophycocyanin (APC)-conjugated anti-c-Kit antibodies (PharMingen). Biotinylated antibodies were detected with streptavidin—Texas Red (Molecular Probes, Eugene, Oreg.). Four-color analysis and sorting were performed on a FACS Vantage (Becton Dickinson, San Jose, Calif.).

Transduction of CD34−KSL Cells

The murine Bmi-1 and Mph-1 cDNAs were FLAG-tagged at their amino-terminus. The retroviral vector GCDNsam (pGCDNsam), with an LTR derived from MSCV, has intact splice donor and splice acceptor sequences for generation of subgenomic mRNA (Kaneko, S. et al., Hum. Gene Ther. 12, 35-44. 2001). Murine Bmi-1, Mph-1, M33, and Bcl-xL cDNAs were subcloned into a site upstream of an IRES-EGFP construct in pGCDNsam. To produce recombinant retrovirus, plasmid DNA was transfected into 293 gp cells (293 cells containing the gag and pol genes but lacking an envelope gene) along with a VSV-G expression plasmid by CaPO4 coprecipitation. Supernatants from transfected cells were concentrated by centrifugation at 6,000 g for 16 hs, then resuspended in α-MEM supplemented with 1% FCS (1/200 of the initial volume of supernatant) Virus titers were determined by infection of Jurkat cells (a human T cell line). CD34−KSL cells were deposited into recombinant fibronectin fragment (Takara Shuzo, Otsu, Japan)-coated 96-well micro-titer plates at 50 to 150 cells per well, and were incubated in α-MEM supplemented with 1% FCS, 100 ng/ml mouse stem cell factor (SCF), 100 ng/ml human thrombopoietin (TPO) (Peprotech, Rocky Hill, N.J.) for 24 hours. Then cells were transduced with a retrovirus vector at a multiplicity of infection (MOI) of 600 in the presence of protamine sulfate (5 μg/ml; Sigma, St. Louis, Mo.) for 24 hours. After transduction, cells were further incubated in S-Clone SF-03 (Sanko Junyaku, Tokyo, Japan) supplemented with 1% FBS, 100 ng/ml SCF, and 100 ng/ml TPO and subjected to in vitro colony assay or competitive repopulation assay at the indicated time point. In all experiments, transduction efficiency was over 80% as judged from the GFP expression observed under a fluorescent inverted microscope.

Colony Assay

CD34⁻KSL cells transduced with the indicated retroviruses, were plated in methylcellulose medium (Stem Cell-Technologies, Vancouver, BC) supplemented with 20 ng/ml mouse SCF, 20 ng/ml mouse IL-3 (Peprotech), 50 ng/ml human TPO, and 2 unit/ml human erythropoietin (EPO) (Peprotech). The culture dishes were incubated at 37° C. in a 5% CO₂ atmosphere. GFP+ colony numbers were counted at day 14. Colonies derived from HPP-CFCs (colony diameter >1 mm) were recovered, cytospun onto glass slides, then subjected to May-Grüenwald Giemsa staining for morphological examination.

Paired Daughter Cell Assay

CD34⁻KSL cells were clonally deposited into 96-well micro-titer plates in S-Clone SF-03 supplemented with 0.1% BSA, 100 ng/ml SCF and 100 ng/ml TPO. When a single cell underwent cell division and gave rise to two daughter cells, daughter cells were separated into different wells by micromanipulation techniques as previously described (Suda, T., et al., Proc. Natl. Acad. Sci. USA 81, 2520-2524, 1984, 1984; Takano, H., et al., J. Exp. Med. 199, 295-302, February 2004). Individual paired daughter cells were further incubated in S-Clone SF-03 supplemented with 10% FCS, 20 ng/ml SCF, 20 ng/ml IL-3, 50 ng/ml TPO, and 2 unit/ml EPO. The colonies generated from each daughter cell were recovered for morphological examination. To evaluate the effect of Bmi-t on HSC fate, CD34⁻KSL cells were transduced with a Bmi-1 retrovirus as described above. After 24 hr transduction, cells were separated clonally by micromanipulation into 96-well micro-titer plates. When a single cell underwent cell division, daughter cells were separated again by micromanipulation and were processed as described above.

Competitive Repopulation Assay

Competitive repopulation assay was performed using the Ly5 congenic mouse system. In brief, hematopoietic cells from B6-ly5.2 mice were mixed with bone marrow competitor cells (B6-Ly5.1) and were transplanted into B6-ly5.1 mice irradiated at a dose of 9.5 Gy. In the case of Ly5.1 hematopoietic cells, cells were mixed with bone marrow competitor cells (B6-Ly5.2) and were transplanted into B6-ly5.2 mice. Four and 12 weeks after transplantation, peripheral blood cells of the recipients were stained with biotinylated anti-Ly5.1 (A20) and FITC-conjugated anti-Ly5.2 (104) (PharMingen). The cells were simultaneously stained with PE-Cy5.5-conjugated anti-B220 antibody, a mixture of APC-conjugated anti-Mac-1 and -Gr-1 antibodies, and a mixture of PE-conjugated anti-CD4 and anti-CDB antibodies (PharMingen). The biotinylated antibody was detected with streptavidin-Texas Red. Cells were analyzed on a FACS Vantage. Percentage chimerism was calculated as (percent donor cells)×100/(percentage donor cells+percent recipient cells). When percent chimerism was above 10 with myeloid, B and T lymphoid lineages, recipient mice were considered to be multilineage reconstituted (positive mice). Repopulation unit (RU) was calculated using Harrison's method (Harrison et al, 1993) as follows: RU=(percent donor cells)×(number of competitor cells)×10⁻⁵/100−(percent donor cells). By definition each RU represents the repopulating activity of 1×10⁵ BM cells. In this example, the number of BM competitors was fixed as 2×10⁵ cells. T/C ratio defined above was applied to Harrison's formula as follows: RU=T/C ratio×2.

RT-PCR

Semi-quantitative RT-PCR was carried out using normalized cDNA by the quantitative PCR using TaqMan rodent GAPDH control reagent (Perkin-Elmer Applied Biosystem, Foster City, Calif.) as described before (Osawa, M., et al., Blood, 100, 2769-2777, 2002). PCR products were separated on agarose gels and visualized by ethidium bromide staining.

(Results)

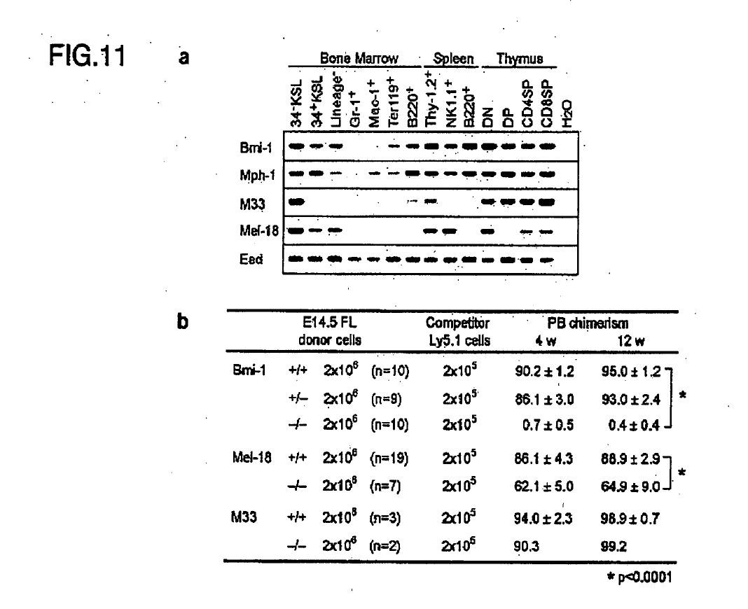

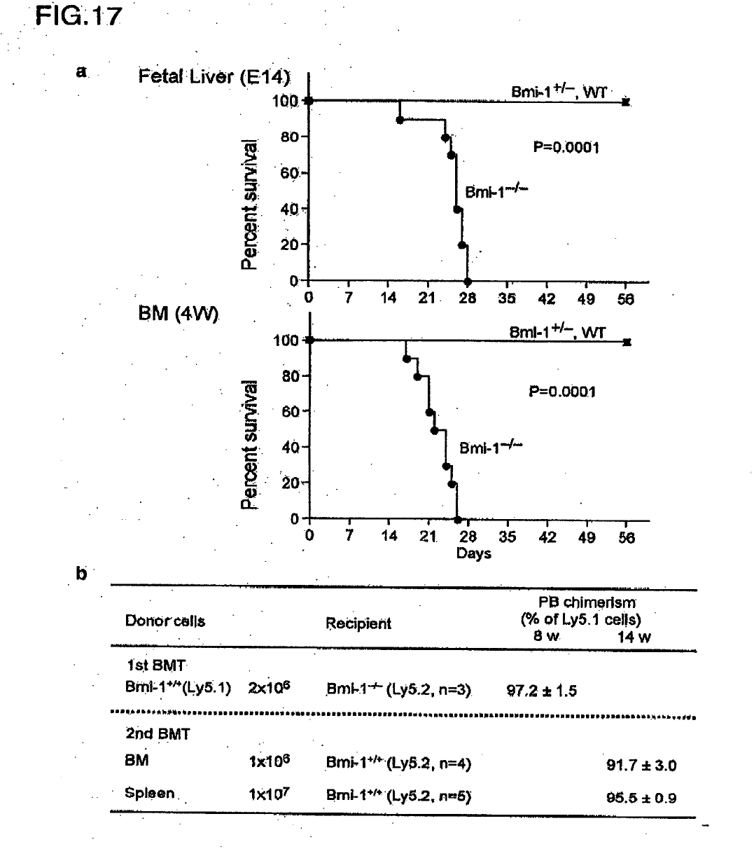

The role of different components of the Bmi-1-containing complex in HSC Expression analysis of PcG genes in human hematopoietic cells has demonstrated that Bmi-1 is preferentially expressed in primitive cells, while other PcG genes, including M33, Mel-18, and Mph1/Rae-28 are not detectable in primitive cells but up-regulated along with differentiation (Lessard, J., et al., Genes & Dev. 13, 2691-2703, 1999). Our detailed RT-PCR analysis of mouse hematopoietic cells, however, revealed that all PcG genes encoding components of the Bmi-1-containing complex, such as Bmi-1, Mph1/Rae-28, M33, and Mel-18, are highly expressed in CD34−KSL HSCs that comprise only 0.004% of bone marrow mononuclear cells (Osawa, M., et al., Science 273, 242-245, 1996), and all are down-regulated during differentiation in the bone marrow (BM) (FIG. 11a). In contrast, Eed, whose product composes another PcG complex, was ubiquitously expressed. These expression profiles support the idea of positive regulation of HSC self-renewal by the Bmi-1-containing complex (Park, I.-K., et al., Nature 423, 302-305., 2003; Lessard, J. et. al., Nature 423, 255-260, 2003). To evaluate the role of uncharacterized PcG components, Mel-18 and M33, in the maintenance of HSCs, we performed competitive repopulation assay using 10 times more fetal liver cells from Bmi-1^{-/-}, Mel-18^{-/-}, and M33^{-/-} mice than competitor cells. As reported, Bmi-1^{-/-} fetal liver cells did not contribute at all to long-term reconstitution (FIG. 11b). The profound defect of repopulating activity was confirmed in a radioprotection assay, in which neither fetal liver cells nor BM cells from Bmi-1^{-/-} mice radioprotected lethally irradiated recipient mice (FIG. 17a). Mel-18 is highly related to Bmi-1 in domain structure, particularly in their N-terminal RING finger and helix-turn-helix (HTH) domains. Unexpectedly, Mel-18^{-/-} fetal liver cells showed a very mild deficiency in repopulating capacity when compared to Bmi-1^{-/-} fetal liver cells (FIG. 11b). Moreover, M33^{-/-} fetal liver cells exhibited normal repopulating capacity (FIG. 11b). As is the case with Bmi-1^{-/-} fetal livers, both Mel-18^{-/-} and M33^{-/-} fetal livers did not show any gross abnormalities, including numbers of hematopoietic cells. To examine the Bmi-1^{-/-} hematopoietic microenvironment, wild type BM cells were transplanted into sub-lethally irradiated Bmi-1^{-/-} mice. Subsequent secondary transplantation exhibited that both Bmi-1^{-/-} BM and spleen can support long-term lymphohematopoiesis, indicating again an intrinsic defect of Bmi-1^{-/-} HSCs (FIG. 17b).

### Example 13

### Defective Self-Renewal and Accelerated Differentiation of Bmi-1^{-/-} HSCs

The present Example demonstrates defective self-renewal and accelerated differentiation of Bmi-1^{-/-} HSCs. The materials and methods were the same as described in Example 12.

(Results)

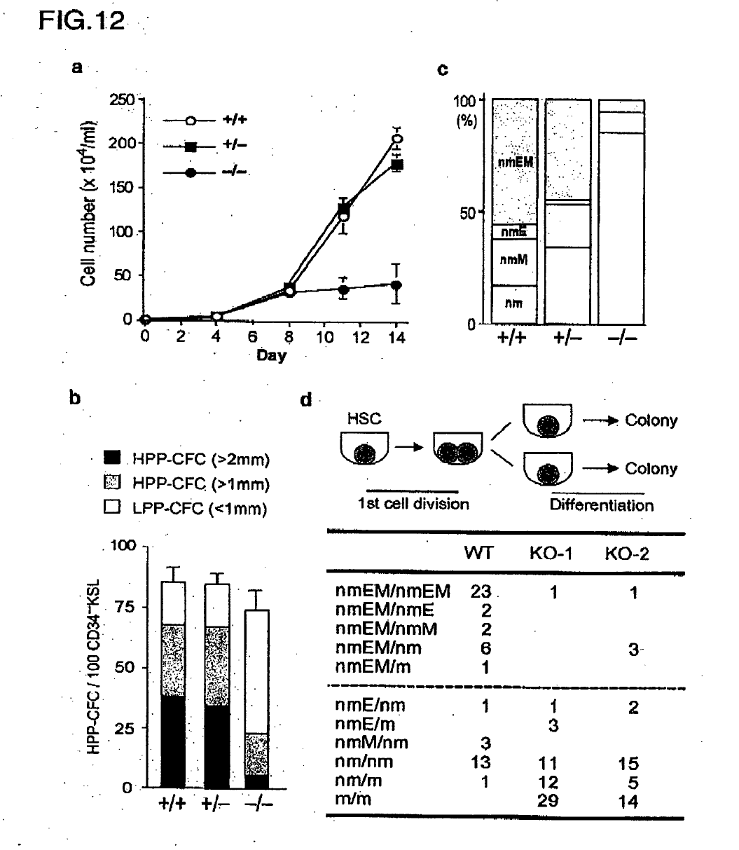

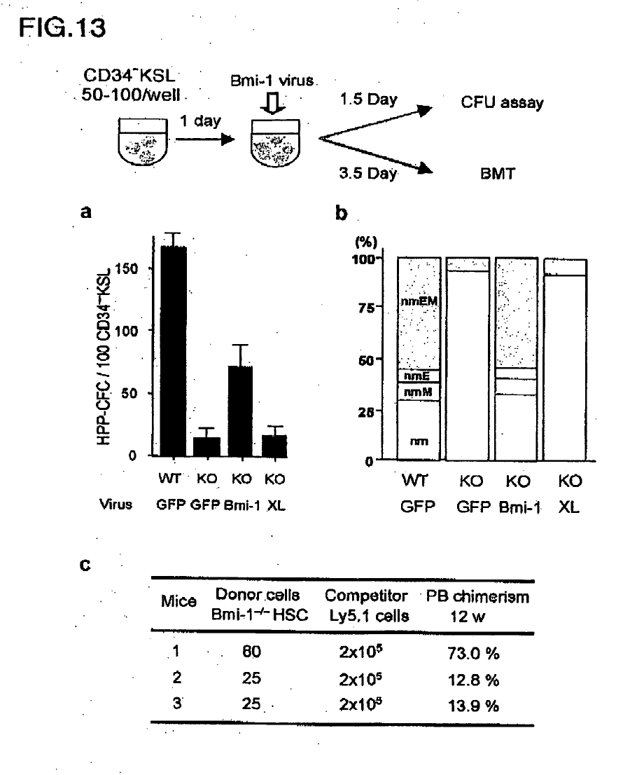

Defective self-renewal and accelerated differentiation of Bmi-1^{-/-} HSCs A progressive postnatal decrease in the number of Thy1.1lowc-Kit+Sca-1+Lineage marker—HSC has been observed in Bmi-1^{-/-} mice (park, I.-K., et al., Nature 423, 302-305, 2003). We also observed approximately 10-fold fewer total CD34−KSL HSCs as measured by flow cytometry in 8-wk-old Bmi-1^{-/-} mice. To evaluate the proliferative and differentiation capacity of Bmi-1^{-/-} HSCs in BM, we purified the CD34⁻KSL HSC fraction, which is highly enriched for long-term repopulating HSCs (Osawa, M., et al., Science 273, 242-245, 1996). Bmi-1^{-/-} CD34⁻KSL cell showed comparable proliferation with wild type and Bmi-1^{+/-} cells for the first week of culture, but thereafter, they proliferated poorly (FIG. 12a). Single cell growth assays demonstrated that Bmi-1^{-/-} CD34⁻-KSL cells are able to form detectable colonies at a frequency comparable to Bmi-1^{+/+} and Bmi-1^{+/-} CD34⁻KSL cells, but contained 3-fold fewer high proliferative potential-colony forming cells (HPP-CFCs). Reduction of HPP-CFCs that gave rise to colonies larger than 2 mm in diameter was even more prominent (7-fold) (FIG. 12b). All HPP colonies larger than 1 mm in diameter were evaluated morphologically. Surprisingly, most of the HPP colonies generated from Bmi-1^{-/-} CD34⁻KSL cells consisted of only neutrophils and macrophages. Bmi-1^{-/-} CD34⁻KSL cells presented a 9-fold reduction in their frequency of colony-forming unit-neutrophil/macrophage/Erythroblast/Megakaryocyte (CFU-nmEM), which retains multi-lineage differentiation capacity, compared with Bmi-1^{-/-} CD34⁻KSL cells (FIG. 12c), Failure of Bmi-1^{-/-} CD34⁻KSL cells to inherit multi-lineage differentiation potential through successive cell division was obvious in a paired daughter assay (FIG. 12d). In most daughter cell pairs generated from Bmi-1^{+/+} CD34⁻KSL cells, at least one of the two daughter cells inherit nmEM differentiation potential, whereas Bmi-1^{-/-} CD34⁻KSL cells showed accelerated loss of multi-lineage differentiation potential, leading to the limited differentiation and inefficient expansion of their progeny. In terms of differentiation, no apparent differentiation block has been observed in Bmi-1^{-/-} lymphocytes despite their reduced numbers (Jacobs, J. J. L., et al., Nature 397, 164-168, 1999). Analysis of myeloid progenitors in BM did not detect any proportional deviations of common myeloid progenitors (CMP), granulocyte/macrophage progenitors (GMP), or megakaryocyte/erythroid progenitors (MEP), either (FIG. 1B), indicating that abnormal hematopoiesis observed in Bmi-1^{-/-} mice does not accompany any specific differentiation block in myeloid lineages. These profound defects of Bmi-1^{-/-} HSC function evoke the possibility that absence of Bmi-1 in HSCs causes additional epigenetic abnormalities that are irreversible, and CD34⁻KSL cells do not retain stem cell properties anymore. Retroviral transduction of Bmi-1^{-/-} CD34⁻KSL cells with Bmi-1, however, completely rescued their defects in proliferation and multi-lineage differentiation potential in vitro (FIGS. 13a and 13b) and long-term repopulating capacity in vivo (FIG. 13c) These findings suggest that execution of stem cell activity is absolutely dependent on Bmi-1. Because Mel-18^{-/-} and M33^{-/-} mice in a C57BL/6 background die during the perinatal period or soon after birth, we could not evaluate their roles in adult BM HSCs.

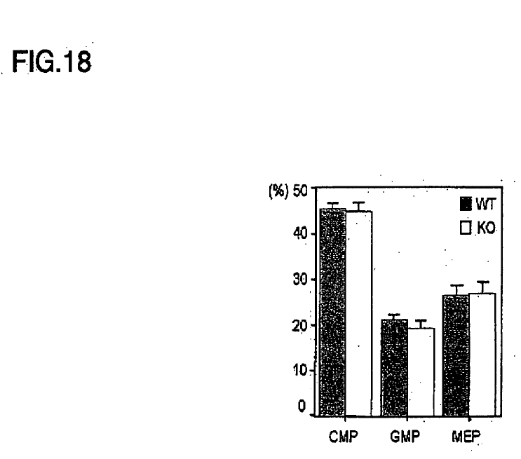

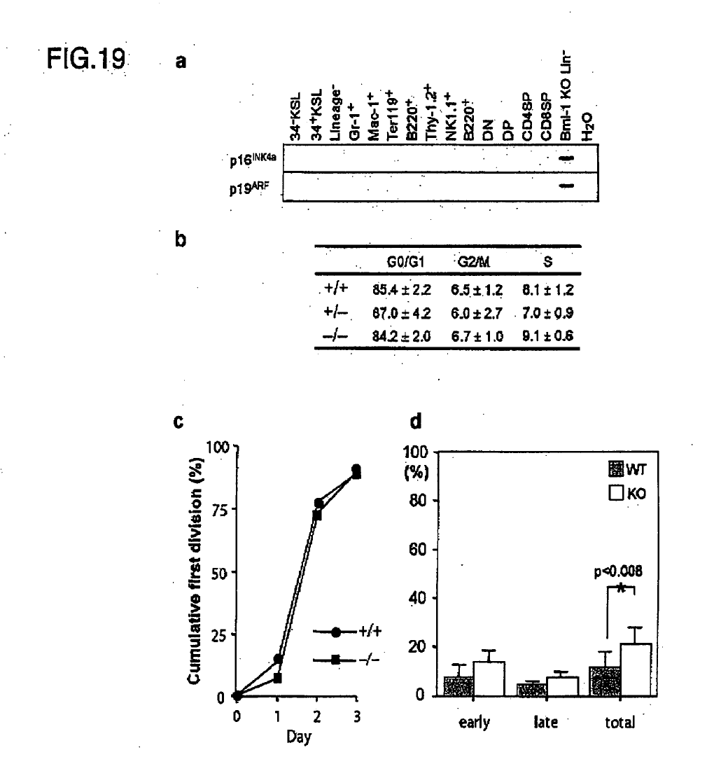

Given the reported involvement of de-repression of p16^{Ink4a} and p19^{Arf} genes in the self-renewal defect in Bmi-1^{-/-} HSCs (Park, I.-K., et al., Nature 423, 302-305, 2003; Lessard, J. et al., Nature 423, 255-260, 2003), we examined their expression in hematopoietic cells. As reported, both were significantly upregulated in Bmi-1^{-/-} Lin cells (FIG. 18a). Overexpression of p16 inhibits G1-S progression, and increased p19 causes p53-dependent growth arrest and apoptosis (Jacobs, J. J. L. et al., Biochim. Biophys. Acta 1602, 151-161,2002; Park, I.-K., et al., Nature 423, 302-305., 2003; Lessard, J. et al., Nature 423, 255-260, 2003). However, cell cycle analysis of Bmi-1^{-/-} BM cells, including KSL primitive progenitors (FIG. 13b), did not discriminate any difference between wild type and Bmi-1^{-/-} mice. Furthermore, in single cell assays, Bmi-1^{-/-} CD34⁻KSL HSCs underwent the first cell division in a fashion similar to that of wild type control (FIG. 18c) and showed no detectable apoptotic cell death), although total Bmi-1^{-/-} BM cells presented a slight but significant increase in apoptotic cell percentage (FIG. 18d). In addition, retrovirally transduced Bcl-xL had no impact on Bmi-1^{-/-} HSCs in vitro (FIGS. 13a and 13b). These findings indicate that de-repression of p16 and p19 genes in Bmi-1^{-/-} HSC does not largely affect the cell cycle or survival of HSCs.

### Example 14

### Augmentation of HSC Activity by Forced Bmi-1 Expression

The present Example shows augmentation of HSC activity by forced Bmi-1 expression. The materials and methods were the same as described in Example 12.

(Results)

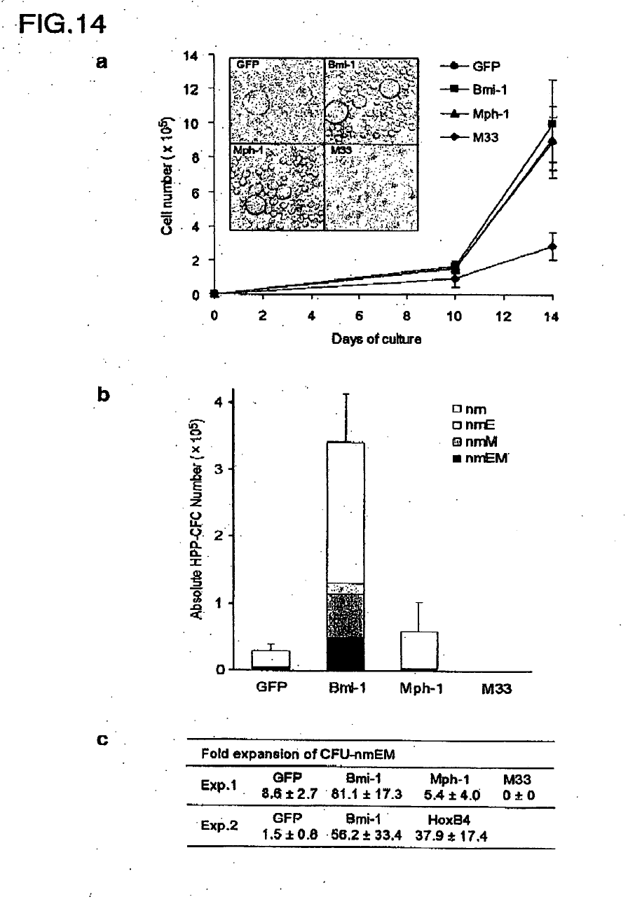

Augmentation of HSC activity by forced Bmi-1 expression An essential role of Bmi-1 in the maintenance of HSC self-renewal capacity prompted us to determine augmentation of HSC activity by PcG genes. CD34⁻KSL HSCs were transduced either with. Bmi-1, Mph1/Rae28, or M33, and then further incubated for 13 days (14-day ex vivo culture in total). Transduction efficiencies were over 80% in all experiments. In the presence of SCF and TPO, which support expansion of HSCs and progenitors rather than their differentiation, forced expression of Bmi-1 as well as Mph1/Rae28 gave no apparent growth advantage in culture compared with the GFP control (FIG. 14a). Notably, however, Bmi-1-transduced but not Mph1/Rae28-transduced cells contained numerous HPP-CFCs (FIG. 14b). Morphological evaluation of the colonies revealed significant expansion of CFU-nmEM by Bmi-1. Given that 60% of freshly isolated CD34⁻KSL cells can be defined as CFU-nmEM as shown in FIG. 12d, there was a net expansion of CFU-nmEM of 56- to 80-fold over 14 days in the Bmi-1 cultures (FIG. 14c). This effect of Bmi-1 is comparable to that of HoxB4, a well-known HSC activator (Antonchuk, J., et al., Cell 109, 39-45, 2002) (FIG. 14c). In addition, both Bmi-1- and HoxB4-transduced cells showed higher proliferative potential and generated much larger colonies compared with the GFP control. Unexpectedly, expression of M33 induced an adverse effect on proliferation and caused accelerated differentiation into macrophages that attached the bottom of culture dishes (FIG. 14a).

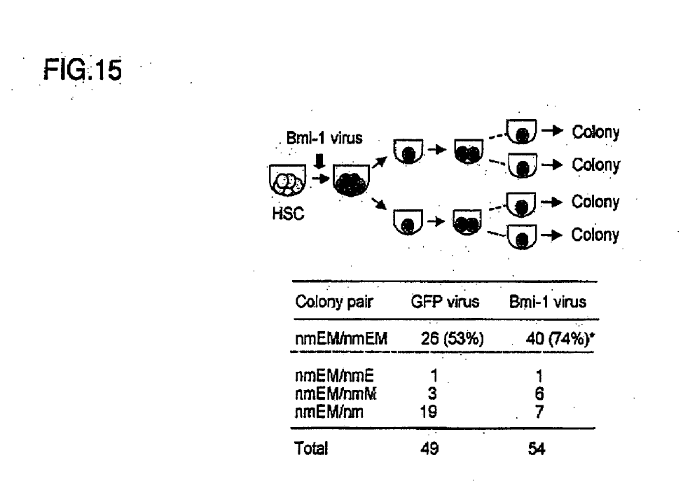

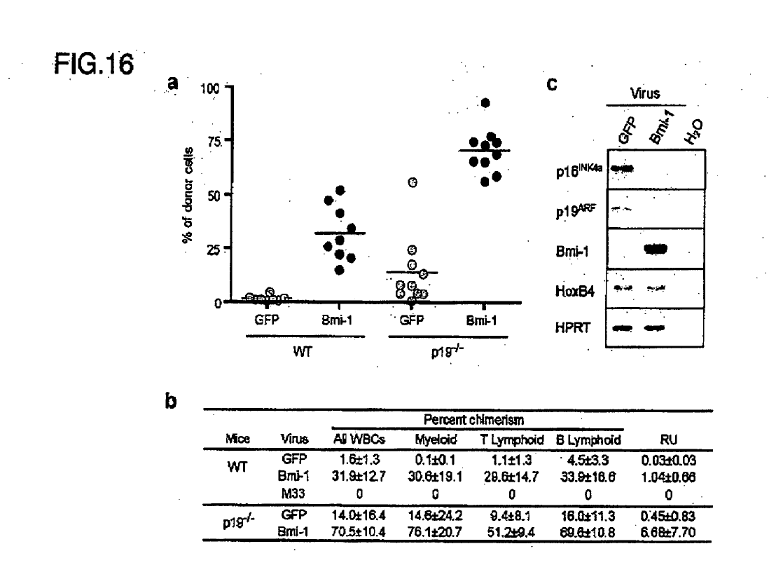

To determine the mechanism that leads to the drastic expansion of CFU-nmEM, which retains a full range of differentiation potential, we employed a paired daughter cell assay to see if overexpression of Bmi-1 promotes symmetric HSC division in vitro. After 24 hr pre-stimulation, CD34⁻KSL cells were transduced with a Bmi-1 expressing retrovirus for another 24 hr. After transduction, single cell cultures were initiated by micromanipulation. When a single cell underwent cell division, the daughter cells were separated again and were allowed to form colonies. To evaluate the commitment process of HSCs while excluding committed progenitors from this invention, we selected daughter cells retaining nmEM differentiation potential by retrospective inference. Expression of Bmi-1 was assessed by GFP expression. As expected, forced expression of Bmi-1 significantly promoted symmetrical cell division of daughter cells (FIG. 15), indicating that Bmi-1 contributes to CFU-nmEM expansion by promoting self-renewal of HSCs We next performed competitive repopulation assays using 10-day ex vivo cultured cells corresponding to 20 initial CD34⁻KSL cells per recipient mouse. After 3 months, mice that received Bmi-1-transduced RSCs demonstrated marked enhancement of multi-lineage repopulation while repopulation mediated by GFP transduced HSCs was barely detectable (FIG. 16a). The repopulating potential in a cell population can be quantitated by calculating repopulation units (RU) from the chimerism of donor cells and the number of competitor cells (Harrison, D. D., et al., Exp. Hematol. 21, 206-219, 1993). Bmi-1-transduced HSCs manifested 35-fold higher RU compared with GFP controls (FIG. 16b). The competitive repopulation assay was similarly performed in parallel using p19^{-/-} HSCs. We expected a drop in Bmi-1-dependent enhancement of repopulation, because p19 is one of the targets negatively regulated by Bmi-1. Nonetheless, expression of Bmi-1 in p19^{-/-} HSC again enhanced multi-lineage repopulation compared with p19^{-/-} GFP control cells (FIG. 16a). A 15-fold increase in RU was obtained with Bmi-1-transduced p19^{-/-} HSCs compared to GFP-transduced p19^{-/-} HSCs (FIG. 16b). This data suggests that p19 is not the main target of Bmi-1 in HSCs. In addition, expression of another Bmi-1 target gene, p16, which is upregulated in ex vivo culture, was completely repressed by Bmi-1 in cultured cells (FIG. 16c). Expression of other cell cycle regulator genes such as INK4 genes (p15INK4b, p18 INK4c, and p19 INK4d) and Cip/Kip genes (p21, p27, and p57) was not grossly affected by Bmi-1 expression. Analysis of percent chimerism of donor cells in each hematopoietic lineages revealed that Bmi-1-transduced HSCs retained full differentiation capacity along myeloid and lymphoid lineages (FIG. 16b). As expected from in vitro data, HSCs transduced with M33 did not contribute to repopulation at all (FIG. 16b).

Discussion

Loss-of-function analyses of the PcG genes Bmi-1 and Mph1/Rae-28 have established that they are essential for the maintenance of adult BM HSCs, but not for the development of definitive HSCs (Ohta, H., et al., J. Exp. Med. 195, 759-770, 2002; Park, I.-K., et al., Nature 423, 302-305., 2003; Lessard, J. et al., Nature 423, 255-260, 2003). Compared with Mph1/Rae-28^{-/-} mice, however, hematopoietic defects are more severe in Bmi-1^{-/-} mice and are attributed to impaired HSC self-renewal (Park, I.-K., et al., Nature 423, 302-305., 2003; Lessard, J. et al., Nature 423, 255-260, 2003). In this invention, we observed normal development of definitive hematopoiesis also in Mel-18^{-/-} and M33^{-/-} fetal livers. Although both Mel-18 and M33 genes are highly expressed in HSCs (FIG. 11a), Mel-18^{-/-} and M33^{-/-} HSCs showed mild or no defects and retained long-term repopulating capacity (FIG. 11b). Accordingly, overexpression of PcG genes in HSCs demonstrated that only Bmi-1 enhances HSC function, while M33 completely abolishes HSC function (FIG. 14 and FIG. 16). All these findings clearly address a central role for Bmi-1 in the maintenance of HSC and suggest that the level of Bmi-1 protein is a critical determinant for the activity of the PcG complex in HSC. Bmi-1 may behave as a core component of the PcG complex in recruiting molecules essential for gene silencing, or provide a docking site for DNA-binding proteins, such as Plzf on HoxD gene regulatory elements (Barna, M., et al., Dev. Cell 3, 499-510., 2002), and E2F6 that targets multimeric chromatin modifiers to E2F- and Myc-responsive genes (Trimarchi, J. M., et al., Proc. Natl. Acad. Sci. USA. 98, 1519-24, 2001; Ogawa, H., et al., Science 296, 1132-1136, 2002). On the other hand, the finding that M33 is dispensable in the maintenance of definitive HSC is surprising. Both Bmi-1 and M33 are involved in the maintenance of homeotic gene expression pattern through development, and strong dosage interactions between the two genes have been observed in this process (Bel, S., et al., Development 125, 3543-3551, 1998). Our finding, however, presents a possibility that M33 does not contribute to the Bmi-1 PCG complex in HSC. M33 could be recruited to histone H3 Lysine 27 methylated by the Eed-containing complex and thereby mediate targeting of the Bmi-1-containing complex to PcG targets (Fischle, W., et al., Genes & Dev. 17, 1870-1881, 2003). Thus, M33 is a key molecule for coordinated regulation of Hox genes by Ead- and Bmi-1-containing complexes. In contrast, the dispensable role of M33 in HSC correlated well to the reciprocal roles of the two complexes in definitive hematopoiesis (Lessard, J., et al., Genes & Dev. 13, 2691-2703, 1999) and indicates that Bmi-1-containing complex has a silencing pathway of its own, The negative effect of overloaded M33 on HSCs could be due to squelching of PcG components by M33. HSCs are maintained and expanded through self-renewal. HSC self-renewal secures its high repopulation capacity and multi-lineage differentiation potential through cell division. If HSCs fail to self-renew, they differentiate to lower orders of progenitors with limited proliferative and differentiation potential. Paired daughter cell assays that monitor the behavior of HSCs in vitro (Suda, T., et al., Proc. Natl. Acad. Sci. USA 81, 2520-2524, 1984, 1984; Takano, H., et al., J. Exp. Med. 199, 295-302, February 2004) demonstrated that Bmi 1 is essential for CD34⁻KSL cells to inherit multi-lineage differentiation potential through successive cell divisions (FIG. 12d). Notably, overexpression of Bmi-1 in CD34⁻KSL cells promoted their symmetrical cell division, indicating a higher probability of inheritance of sternness mediated by Bmi-1 (FIG. 15). This is the first evidence of successful genetic manipulation of HSC self-renewal in vitro. These clonal observations together with functional rescue of Bmi-1^{-/-} HSC both in vitro and in vivo strongly support an essential role of Bmi-1 in HSC self-renewal. In the process of proliferation and differentiation of HSCs, progenitor expansion occurs at each progenitor level. Bmi-1^{-/-} HSCs showed a very low proliferative potential. On the contrary, forced expression of Bmi-1 conferred high proliferative potential to HSCs through MPP expansion (FIG. 14). These findings indicate that Bmi-1 is essential not only to HSC self-renewal but also to progenitor expansion. The central role for Bmi-1 in HSC self-renewal was also demonstrated by overexpression experiments of PcG genes in HSC. The Bmi-1-mediated growth advantage was largely restricted to the primitive hematopoietic cells. During ex vivo culture, total cell numbers were almost comparable to the control while a net 56- to 80-fold CFU-nmEM expansion and 15- to 35-fold higher repopulation activity were obtained in the Bmi-1 cultures (FIGS. 4 and 6). In agreement with these data, symmetrical cell division of HSC was promoted in the Bmi-1 cultures (FIG. 15). These observations suggest enhanced probability of HSC self-renewal and progenitor expansion mediated by Bmi-1 overexpression. Although Bmi-1-transduced HSC established higher repopulation in vivo, chimerism of Bmi-1-transduced HSC progenies reached its plateau between 2 to 3 months and never showed continuous growth advantages in vivo. This could be due to silencing of retroviral Bmi-1 expression in vivo as suggested by a significant decrease in GFP intensity detected by flow cytometric analysis (data not shown). Thus, marked enhancement of HSC repopulating capacity might be obtained by enhanced HSC recovery after ex vivo culture. Alternatively, increased expression of Bmi-1 may not confer a growth advantage in steady state hematopoiesis once HSC becomes quiescent in the niche. The comparable effect of Bmi-1 to that of HoxB4, a well-known HSC activator (Antonchuk, J., et al., Cell 109, 39-45, 2002), is noteworthy. Recent findings indicated that genetic manipulation of HoxB4 can support generation of long-term repopulating HSCs from ES cells (Kyba, M., et al., Blood 91, 1216-1224, 1998), and ex vivo expansion of HSCs can be obtained by direct targeting of HoxB4 protein into HSCs (Amsellem, S., et al., Nat. Med. 9, 1423-1427, 2003; Krosl, J., et al., Nat. Med. 9, 1428-1432, 2003). Similar to HoxB4, Bmi-1 could be a novel target for therapeutic manipulation of HSCs. Although PcG proteins regulate expression of homeotic genes including HoxB4 during development (Takihara, Y., et al., Development 124, 3673-3682, 1997), de-regulation of Hox genes in definitive hematopoietic cells have not yet been identified in mice deficient for PcG genes (Ohta, H., et al., J Exp. Med. 195, 759-770, 2002; Park, I.-K., et al., Nature 423, 302-305., 2003; Lessard, J. et al., Nature 423, 255-260, 2003). In this invention, HoxB4 expression was not altered in Bmi-1-overexpressing hematopoietic cells, either (FIG. 16c). Nevertheless, the enhancement of HSC activity by two genes is highly similar in many aspects. It will be intriguing to ask how these two genes work as HSC activators and whether there is cross talk between these two pathways or not. The mechanism whereby Bmi-1 maintains HSC remains to be defined. Although de-repression of Bmi-1 target genes, p16 and p19 has been attributed to defective HSC self-renewal, the cell cycle status of CD34−KSL. HSCs was not grossly altered in Bmi-1^{-/-} mice (FIG. 18). In addition, apoptosis was not increased during observation of clonal HSC cultures, either. Therefore, a detailed analysis of Bmi-1^{-/-}p16^{-/-}p19^{-/-} HSCs will be necessary to define their roles in HSC. Nonetheless, p19^{-/-} HSCs showed higher repopulating capacity than wild type control (FIGS. 16a and 16b), and enhanced HSC repopulating capacity mediated by Bmi-1 was correlated with repressed p16 and p19 expression in ex vivo cultured HSCs (FIG. 16e). One attractive hypothesis is that de-repression of p16 and p19 genes causes early senescence of primitive hematopoietic cells as reported in Bmi-1^{-/-} mouse embryonic fibroblasts (Jacobs, J. J. L., et al., Nature 397, 164-168, 1999). In the case of multipotent hematopoietic cells, senescence could mean accelerated differentiation and early cell cycle exit as observed in Bmi-1^{-/-} mice. In BM, HSCs reside in a niche in close contact with supporting cells like osteoblasts (Zhang, J., et al., Nature 425, 836-841, 2003; Calvi, L. M., et al., Nature 425, 841-846, 2003), in which most of the HSCs stay in the G0 stage. The quiescence of HSCs has a critical biological importance in preventing premature HSC exhaustion (Cheng, T., et al., Science 287, 1804-1808, 2000). Taken together, HSC sternness might be maintained by a fine regulation of the cell cycle machinery. Additional mechanisms regulating self-renewal could be responsible for preventing differentiation (Wang, Z. et al., Science 303, 2016-2019, March 2004). We found that Bmi-1 inhibits differentiation of an immature hematopoietic cell line. It is well recognized that HSCs express most myeloid genes at a low level (Miyamoto, T., et al., Dev. Cell 3, 137-147, 2002). Bmi-1 in HSC might be involved in repressing differentiation-related gene expression below the level of biological significance. The increase in Bmi-1 expression may mediate many if not all of the phenotypic changes in C/EBPα^{-/-} HSCs and may also mediate some of the block in myeloid differentiation observed in C/EBPα^{-/-} mice. Further analysis of the underlying mechanisms in Bmi-1^{-/-} cells will be needed to unveil the relative contributions of Bmi-1 to self-renewal and/or differentiation. Finally, however, since disruption of C/EBPα has been described in a number of humans with Acute Myeloid Leukemia, it will also be of interest to investigate whether Bmi-1 is upregulated in the leukemic blasts, and whether such upregulation contributes to the self-renewal function of leukemic stem cells, which is defective in experimental models of leukemia in cells lacking Bmi-1 (Lessard, J. et al., Nature 423, 255-260, 2003).

Although certain preferred embodiments have been described herein, it is not intended that such embodiments be construed as limitations on the scope of the invention except as set forth in the appended claims. All patents, published patent applications and publications cited herein are incorporated by reference as if set forth fully herein.

### Industrial Applicability

The present invention provides an agent capable of effectively regulating the expansion of a hematopoietic stem cell, which could not be conventionally achieved. Accordingly, it is possible to regulate the expansion of a hematopoietic stem cell, which is difficult in conventional situations, thereby making it possible to use the present invention for diseases involved in an abnormality of a hematopoietic cell.

Various other modifications will be apparent to and can be readily made by those skilled in the art without departing from the scope and spirit of this invention. Accordingly, it is not intended that the scope of the claims appended hereto be limited to the description as set forth herein, but rather that the claims be broadly construed.

1. A method for regulating expansion of a stem cell, comprising the steps of:
(A) providing, to the stem cell, Bmi-1 or a variant or fragment thereof and/or a Bmi-1 regulating agent in an amount sufficient for regulation of expansion of the hematopoietic stem cell; and
(B) culturing the stem cell for a time sufficient for the regulation of expansion.

2. A method according to claim 1, wherein the regulation of expansion is promotion of expansion.

3. A method according to claim 1, wherein the Bmi-1 or a variant or fragment thereof and/or the Bmi-1 regulating agent are exogenous or endogenous.

4. A method according to claim 1, wherein the Bmi-1 or a variant or fragment thereof and/or the Bmi-1 regulating agent are exogenous.

5. A method according to claim 1, wherein the Bmi-1 or a variant or fragment thereof and/or the Bmi-1 regulating agent is in the form of a nucleic acid and/or a protein.

6. A method according to claim 1, wherein the Bmi-1 or a variant or fragment thereof and/or the Bmi-1 regulating agent include:
(a) a polypeptide encoded by a nucleic acid sequence as set forth in SEQ ID NO:1 or 3 (Accession No. L13689 or M64279, respectively) or a fragment thereof;
(b) a polypeptide having an amino acid sequence as set forth in SEQ ID NO:2 or 4 or a fragment thereof;
(c) a variant polypeptide having an amino acid sequence as set forth in SEQ ID NO:2 or 4 having at least one amino acid mutation selected from the group consisting of substitutions, additions, and deletions, the variant polypeptide having a biological activity; or
(d) a polypeptide having at least 70% amino acid sequence homology to any one of polypeptides (a) to (c) and having biological activity.

7. A method according to claim 1, wherein the Bmi-1 or a variant or fragment thereof and/or the Bmi-1 regulating agent include:
(a) a polynucleotide having a base sequence as set forth in SEQ ID NO:1 or 3 (Accession No L13689 or M64279, respectively) or a fragment thereof;
(b) a polynucleotide encoding an amino acid sequence as set forth in SEQ ID NO:2 or 4 or a fragment thereof;
(c) a polynucleotide encoding a variant polypeptide having an amino acid sequence as set forth in SEQ ID NO:2 or 4 having at least one-amino acid mutation consisting of substitutions, additions, and deletions, and having biological activity;
(d) a polynucleotide encoding a polypeptide hybridizable to any one of the polynucleotides of (a) to (c) under stringent conditions; or
(e) a polynucleotide encoding a polypeptide having a base sequence having at least 70% identity to any one of the polynucleotides of (a) to (c) or a complementary sequence thereof and having biological activity.

8. A method according to claim 1, wherein the Bmi-1 or a variant or fragment thereof and/or the Bmi-1 regulating agent are selected from the group consisting of a small molecule, a lipid molecule, a sugar, and a complex thereof.

9. A method according to claim 1, wherein the stem cell includes a stem cell selected from the group consisting of hematopoietic stem cells, germ line stem cells, and neural stem cells.

10. A method for regulating a disease, disorder, or abnormality related to hematopoiesis, reproduction, or a nervous system, comprising the steps of:
(A) providing, to a subject, Bmi-1 or a variant or fragment thereof and/or a Bmi-1 regulating agent in an amount sufficient for regulation of expansion of a stem cell; and
(B) allowing the subject sufficient time for regulation of expansion to occur.

11. A composition for regulating expansion of a stem cell, comprising Bmi-1 or a variant or fragment thereof and/or a Bmi-1 regulating agent in an amount sufficient for regulation of expansion.

12. A composition according to claim 11, wherein the regulation of expansion is promotion of expansion.

13. A composition according to claim 11, wherein the stem cell includes a stem cell selected from the group consisting of hematopoietic stem cells, germ line stem cells, and neural stem cells.

14. A composition according to claim 11, wherein the Bmi-1 or a variant or fragment thereof and/or the Bmi-1 regulating agent include:
(a) a polypeptide encoded by a nucleic acid sequence as set forth in SEQ ID NO:1 or 3 (Accession No. L13689 or M64279, respectively) or a fragment thereof;
(b) a polypeptide having an amino acid sequence as set forth in SEQ ID NO:2 or 4 or a fragment thereof;
(c) a variant polypeptide having an amino acid sequence as set forth in SEQ ID NO:2 or 4 having at least one amino acid mutation selected from the group consisting of substitutions, additions, and deletions, the variant polypeptide having a biological activity; or
(d) a polypeptide having at least 70% amino acid sequence homology to any one of polypeptides (a) to (c) and having biological activity.

15. A composition according to claim 11, wherein the Bmi-1 or a variant or fragment thereof and/or the Bmi-1 regulating agent include:
(a) a polynucleotide having a base sequence as set forth in SEQ ID NO:1 or 3 (Accession No. L13689 or M64279, respectively) or a fragment thereof:
(b) a polynucleotide encoding an amino acid sequence as set forth in SEQ ID NO:2 or 4 or a fragment thereof;
(c) a polynucleotide encoding a variant polypeptide having an amino acid sequence as set forth in SEQ ID NO:2 or 4 having at least one amino acid mutation consisting of substitutions, additions, and deletions, and having biological activity;
(d) a polynucleotide encoding a polypeptide hybridizable to any one of the polynucleotides of (a) to (c) under stringent conditions; or
(e) a polynucleotide encoding a polypeptide having a base sequence having at least 70% identity to any one of the polynucleotides of (a) to (c) or a complementary sequence thereof and having biological activity.

16. A composition according to claim 11, wherein the Bmi-1 complexes with Mph-1/Rae28 or M33.

17. A composition according to claim 16, wherein the Bmi-1 complexes with Mph-1/Rae28 and M33.

18. A composition according to claim 11, wherein the Bmi-1 or a fragment or variant thereof and/or the Bmi-1 regulating agent is consistently active.

19. A composition according to claim 11, wherein the Bmi-1 or a fragment or variant thereof and/or the Bmi-1 regulating agent is transiently active.

20. A composition according to claim 11, wherein the Bmi-1 consists of a sequence as set forth in SEQ ID NO:1 (Accession No. L13689).

21. A composition according to claim 11, further comprising a cellularly phisiologically active substance.

22. A composition according to claim 21, wherein the cellularly phisiologically active substance comprises an agent selected from the group consisting of SCF, TPO, and Flt-3L.

23. A composition according to claim 11, further comprising a pharmaceutically acceptable carrier.

24. A composition according to claim 11, wherein the Bmi-1 regulating agent comprises an agent capable of activating the Bmi-1.

25. A composition according to claim 11, further comprising an agent capable of binding to an agent capable of increasing an activity of a promoter of Bmi-1 or a PcG complex comprising Bmi-1 to enhance a function thereof.

26. A composition according to claim 11, wherein the Bmi-1 or a fragment or variant thereof and/or the Bmi-1 regulating agent is in the form of a protein or a complexed protein.

27. A composition according to claim 11, wherein the Bmi-1 or a fragment or variant thereof and/or the Bmi-1 regulating agent is in the form of a nucleic acid.

28. A composition according to claim 27, wherein the Bmi-1 or a fragment or variant thereof and/or the Bmi-1 regulating agent in the form of a nucleic acid is contained in a vector.

29. A composition according to claim 28, wherein the vector is a retrovirus vector.

30. A method for treatment or prophylaxis of a disease, disorder, or abnormality related to hematopoiesis, reproduction, or a nervous system, comprising the step of:
administering Bmi-1 or a variant or fragment thereof and/or a Bmi-1 regulating agent in an amount sufficient for the treatment or prophylaxis of a subject requiring regulation thereof.

31. A pharmaceutical composition for treatment or prophylaxis of a disease, disorder, or abnormality related to hematopoiesis, reproduction, or a nervous system, comprising:
Bmi-1 or a variant or fragment thereof and/or a Bmi-1 regulating agent in an amount sufficient for the treatment or prophylaxis.

32. A kit for regulating expansion of a stem cell, comprising:
(A) a composition comprising Bmi-1 or a variant or fragment thereof and/or a Bmi-1 regulating agent in an amount sufficient for regulation of expansion; and
(B) instructions setting forth a method of providing the composition to the stem cell and culturing the hematopoietic stem cell.

33. Use of Bmi-1 or a variant or fragment thereof and/or a Bmi-1 regulating agent for regulating expansion of a stem cell.

34. A method for producing a cell expanded from a stem cell line, comprising the steps of;
(A) providing a stem cell or a primordial cell;
(B) providing, to the stem cell or the primordial cell, Bmi-1 or a variant or fragment thereof and/or a Bmi-1 regulating agent in an amount sufficient for regulation of expansion thereof; and
(C) culturing the stem cell for a time sufficient for the regulation of expansion

35. A method according to claim 34, wherein the stem cell includes a stem cell selected from the group consisting of hematopoietic stem cells, germ line stem cells, and neural stem cells.

36. A cell obtained by a method according to claim 35.

37. A tissue obtained from a cell obtained by a method according to claim 35.

38. An organ obtained from a cell obtained by a method according to claim 35.

39. A medicament, comprising a cell obtained by a method according to claim 35.

40. A method for treatment or prophylaxis of a disease or disorder requiring a stem cell or an expansion cell derived therefrom, comprising the step of:
(A) administering a cell obtained by a method according to claim 35 to a subject requiring the treatment or prophylaxis.

41. Use of a cell obtained by a method according to claim 35, for treatment or prophylaxis of a disease or disorder requiring a stem cell or an expansion cell derived therefrom.

42. A method for screening for an agent for regulating expansion of a stem cells comprising the steps of:
(A) providing candidate substances for the agent:
(B) exposing a cell containing Bmi-1 to the substances; and
(C) determining whether or not the Bmi-1 is regulated, wherein when the Bmi-1 is regulated, the substance is determined to be an agent capable of regulating the expansion of the stem cell.

43. An agent for regulating expansion of a hematopoietic stem cell, obtained by a method according to claim 42.


SEQUENCE LISTING (30 sequences)

SEQ ID NO: 1 - DNA, 3203 bp, Homo sapiens
  CDS (480)..(1460)
  ref <308>: DATABASE ACCESSION NUMBER: L13689
  ref <309>: DATABASE ENTRY DATE: 1994-07-19
  ref <313>: RELEVANT RESIDUES: (1)..(3203)
gagaggcagagatcggggcgagacaatggggatgtgggcgcgggagccccgttccggcttagcagcacctcccagccccgcagaataaaaccgatcgcgccccctccgcgcgcgccctcccccgagtgcggagcgggaggaggcggcggcggccgaggaggaggaggaggaggccccggaggaggaggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacttgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggagttccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttgatacctgagactgttaaggaaaaaaattttaaacccctgatttatatagatatcttcagccattacgactttctagagctaatacatgtgactatcgtccaatttgctttcttttgtagtgacattaaatttggctataaaagatggactacatgtgatactcctgtccgtcttggttcaaaagaaagattgttgttataaagaattggtttcttggaaagcaggcaagactttttctctgtgttaggaaagatgggaaatggtttctgtaaccattgtttggatttggaagtactctgcagtggacataagcattgggccatagtttgttaatctcaactaacgcctacattacattctccttgatcgttcttgttattacgctgttttgtgaacctgtagaaaaacaagtgctttttatcttgaaattcaaccaacggaaagaatatgcatagaataatgcattctatgatgccatgtcactgtgaataacgatttcttgcagctatttagccattttgattgctgtttgatttatacttctctgttgctacgcaaaaccgatcaaagaaaagtgaacttcagttttacaatctgtatgcctaaaagcgggtactaccgtttattttactgacttgtttaaatgattcgcttttgtaagaatcagatggcattatgcttgttgtacaatgccatattggtatatgacataacaggaaacagtattgtatgatatatctataaatgctataaagaaatattgtgtttcatgcattcagaaatgattgttaaaattctcccaactggttcgacctttgcagatacccataacctatgttgagccttgcttaccagcaaagaatatttttaatgtggatatctaattctaaagtctgttccattagaagcaattggcacatctttctatactttatatacttttctccagtaatacatgtttactttaaaaattgttgcagtgaagaaaaacctttaactgagaaatatggaaaccgtcttaattttccattggctatgatggaattaatattgtattttaaaaatgcatattgatcactataattctaaaacaattttttaaataaaccagcaggttgctaaaagaaggcattttatctaaagttattttaataggtggtatagcagtaattttaaatttaagagttgcttttacagttaacaatggaatatgccttctctgctatgtctgaaaatagaagctatttattatgagcttctacaggtatttttaaatagagcaagcatgttgaatttaaaatatgaataaccccacccaacaattttcagtttattttttgctttggtcgaacttggtgtgtgttcatcagttatttgtgagggtgtttattctatatgaatattgtttcatgtttgtagggaaattgtagctaaacatttcattgtccccagtctgcaaaagaagcacaattctattgctttgtcttgcttatagtcattaaatcattacttttacatatattgctgttacttctgctttctttaaaaatatagtaaaggatgttttatgaagtcacaagatacatatatttttattttgacctaaatttgtacagtcccattgtaagtgttgtttctaattatagatgtaaaatgaaatttcatttgtaattggaaaaaatccaataaaaaggatattcatttagaaaatagctaagatctttaataaaaatttgatatgaaa

SEQ ID NO: 2 - protein, 326 aa, Homo sapiens [sequence omitted]

SEQ ID NO: 3 - DNA, 3600 bp, Mus musculus
  CDS (472)..(1446)
  ref <308>: DATABASE ACCESSION NUMBER: M64279
  ref <309>: DATABASE ENTRY DATE: 1993-04-27
  ref <313>: RELEVANT RESIDUES: (1)..(3600)
ataatcgtagtattaaaggcagagatcgggagagacaatggggaggttggcgagggagccccgcgcaggcttagcaacacctcccggccccgcagaataatacggctcggcccctctgcgcgccctcccccgagcgcggccgggaggcggcggctgcgccgaggaggaggagaggccccgaggaggcggcggcggaggccgcgcggaggcggaggaggccgcgcgggaggaggaggccgcagcgcgcgagcaggaggccggcgggggagcggcatgagcggagcgcggcggccgtggctgctctcggccgcgctcggtgcccattgacagcggcggcggcggctcgctccaagatggccgcttggctcgcattcattttatgctgaacgacttttaactttcattgtcttttccgcccgctcagatcgcctcgcgccggccgctctttccgggatcttttatcaagcagaaatgcatcgaacaaccagaatcaagatcactgagctaaatccccacttaatgtgtgtcctgtgtggagggtacttcattgatgccactaccataatagaatgtctacattccttctgtaaaacatgtattgtacgttacttggagaccagcaagtattgtcctatttgtgatgtccaggttcacaaaaccagaccactcctgaacataaggtcagataaaactcttcaagatattgtatacaaattagtcccagggcttttcaaaaatgagatgaagagaagaagagatttttatgcagctcacccgtcagctgatgctgccaatggctccaatgaagaccgaggagaagttgcagatgaggagaagaggattataactgatgatgagataataagcttgtctattgagttctttgatcagagcagattggatcggaaagtaaataaagagaagcctaaggaagaggtgaatgataaaaggtacttacgatgcccagcagcaatgactgtgatgcacttgagaaagtttctcaggagtaaaatggacatacccaatactttccagattgacgtcatgtatgaagaggaacctttaaaggattactacacgctaatggacattgcctacatttatacctggagaagaaatggcccactacctttgaaatacagagttcggccaacttgcaaaagaatgaagatgagtcaccagagggatggactgacgaatgctggagagctggaaagtgactctgggagtgacaaggccaacagcccagccggaggtgttccctccacctcttcctgtttgcccagtcccagcactccagtgcagtctcctcacccacagttccctcacatttccagtaccatgaatggaaccagcaacagccccagtgctaaccaccaatcttcctttgccagtagacctcgaaaatcatcactaaatgggtcatcagcaacttcatctggttaggactgttaaggaaaagatttttcaaccccctgatttagttaccttcattcattacagctttatagatgcttaatacatgtgactgtcgtccagtttgcttccttttgtagtgactttaaatttggccataaatgatggactagatgtgatacttcatatggatgttaagtggaaagattgattctttctctaaagaattggattctgagaaggattctgtgttaggaaagatgtgaaatgatttctgtgaccactgtttggatctggaaatgttctacagtgggtagacattgggccatagtttgttaatctcaattaatgcctacattacattctctttgattgttcttgttattatgctgttctgtgaacctgtagaaaacaagtgctttttatcttgaaattcagcaaatggaaagaataagcatagaatactgcattctgtgcagccacgtcactgtgaataacaatttcttgcatatttagccattttaattcctgtttgatttttacttctctgttgctacacaaaatgatcaaaggaaacttgttttacaatctgtatgcctaaaaagcgggtactaccgtttattttactgacttgttgaaatgattcacttttgtaagaatcagatggcattatgcttgttgtacaatgccatattggtatatgacataacaggaaacagtattgtatgatatatttataaatactatgaaaatattgtgtttcatgcattcaaaaacagttgtaaacttctccaaatgggttcgacctttgcggatatcacagtgcacccaatgctgaggcttaccaccagcacagaatttgttttaatgttgataaccagtttcaaattcttttccacttagaagcaaatggcagatcttcatagttctggcttttctccaataagactttcaaaattaatattgcagtaaagaaaaattttaaccaaggcaaatatagtgtttaattttccattggttaaaatagaattaatgtttttaaattctgtttatcagtggaattctaaaatagattttaaagtcaaccagctaattgctgaagtaaagcattttatctacaattttattttagtgtatgtggcacaagtgtaatttcaaatttaagagttgttttcatagttaaaggaatctgccttcactgtaacgtctggaaacagaaacaatttgttatgatttctacagggatttttaatagagcaaacatgttgaatttaaaatatgaatgacccctccaagtctcagttttgctttggtcgaacttagcgtgtgtttatcagccatcagttatttgtgaggatgttaattccgtatgaatattgtttcatgtttgtatgggaaactgtagctaagcatttcattgtctgcagtctgcagaagaagcacaattgtattgctttgtcttgcttgtagccattaaatcattacttttacacacattgctgttaacttctgctttctttaaagatttagtaaattgatgttttatgaaggccacaagatacatatatttttattttgacctaaatttgtacagtcccattgtgtgtgtgtcatttctaattatagatgtaaaattaaatttcatttttaattggaaaattcaataaaaagatattcatttagaaaatactatgctctttaattaaaattttgctatgaaaagcacagtgtgcagaagttttgggaaaccctgtagtggattgtaagagctaaaacctaaggaaaatactttgttgtcttcaaatgatgttgttagtaaatgctttgttgtatattactcgaaagcccaatgaaagaagtatctgcttatttacaatctttgaagcaaaagttaccaatgtttgccaattttggtgaatttaactcatttttctagacttttctcgaggttttcatggtgttacctaagacaaaagacatctcaccctctatgatgacttacttctgagagtgcgtttgaggcacttatggcttactaagcagtgtgtcaccatacttgaaaactcttccatttattgtatctggatgaggctttttacccttactcaatttgaaaattgcttaagcttaaatgatatttcagtcaaaatttgtcttttaataaaacaacgaaagatgaaaaaa

SEQ ID NO: 4 - protein, 324 aa, Mus musculus
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEEKRIITDDEIISLSIEFFDQSRLDRKVNKEKPKEEVNDKRYLRCPAAMTVMHLRKFLRSKMDIPNTFQIDVMYEEEPLKDYYTLMDIAYIYTWRRNGPLPLKYRVRPTCKRMKMSHQRDGLTNAGELESDSGSDKANSPAGGVPSTSSCLPSPSTPVQSPHPQFPHISSTMNGTSNSPSANHQSSFASRPRKSSLNGSSATSSG

SEQ ID NO: 5 - DNA, 2227 bp, Homo sapiens
  CDS (202)..(1236)
  ref <308>: DATABASE ACCESSION NUMBER: NM_007144
  ref <309>: DATABASE ENTRY DATE: 2003-09-08
  ref <313>: RELEVANT RESIDUES: (1)..(2227)
gagagcccgaacaggaagagggtacagctttgtgcaggtcacatgcccactgcagccctccagcctctggtccccagagcggactttggaagctgaactgcttttgttgctggaagacttatgttataatttaccctgggtggaccagggtcgtacaaaagggcaacgctccccagtccccccactcccgaccccggaatcatgcatcggactacacggatcaaaatcacagagctgaacccccacctcatgtgtgccctctgcggggggtacttcatcgacgccaccactatcgtggagtgcctgcattccttctgcaaaacctgcatcgtgcgctacctggagaccaacaaatactgccccatgtgtgacgtgcaggtccataaaacccggccgctgctgagcatcaggtctgacaaaacacttcaagacattgtctacaaattggtccctgggctttttaaagatgagatgaaacggcggcgggatttctatgcagcgtaccccctgacggaggtccccaacggctccaatgaggaccgcggcgaggtcttggagcaggagaagggggctctgagtgatgatgagattgtcagcctctccatcgaattctacgaaggtgccagggaccgggatgagaagaagggccccctggagaatggggatggggacaaagagaaaacaggggtgcgcttcctgcgatgcccagcagccatgaccgtcatgcatcttgccaagtttctccgcaacaagatggatgtgcccagcaagtacaaggtggaggttctgtacgaggacgagccactgaaggaatactacaccctcatggacatcgcctacatctacccctggcggcggaacgggcctctccccctcaagtaccgtgtccagccagcctgcaagcggctcaccctagccacggtgcccaccccctccgagggcaccaacaccagcggggcgtccgagtgtgagtcagtcagcgacaaggctcccagccctgccaccctgccagccacctcctcctccctgcccagcccagccaccccatcccatggctctcccagttcccatgggcctccagccacccaccctacctcccccactcccccttcgacagccagtggggccaccacagctgccaacgggggtagcttgaactgcctgcagacaccatcctccaccagcagggggcgcaagatgactgtcaacggcgctcccgtgccccccttaacttgaggccagggaccctctcccttcttccagccaagcctctccactccttccactttttctgggcccttttttccacttcttctactttccccagctcttcccaccttgggggtggggggcgggttttataaataaatatatatatatatgtacataggaaaaaccaaatatacatacttattttctatggaccaaccagattaatttaaatgccacaggaaacaaactttatgtgtgtgtgtatgtgtggaaaatggtgttcattttttttggggggggtcttgtgtaatttgctgtttttgggggtgcctggagatgaactggatgggccactggagtctcaataaagctctgcaccatcctcgctgtttcccaaggcaggtggtgtgttgggggccccttcagacccaaagctttaggcatgattccaactggctgcatataggagtcagttagaattgtttctttctctccccgtttctctccccatcttggctgctgtcctgcctctgaccagtggccgccccccgcgttgttgaatgtccagaaattgctaagaacagtgccttttacaaatgcagtttatccctggttctgaggagcaagtgcagggtggaggtggcacctgcatcacctcctcctcttgcagtggaaactttgtgcaaagaatagatagttctgcctcttttttttttttttcctgtgtgtgtggcctttgcatcatttatcttgtggaaaagaagattcaggccctgagaggtctcagctcttggaggagggctaaggctttagcattgtgaagcgctgcacccccaccaaccttaccctcaccggggaaccctcactagcaggactggtggtggagtctcacctggggcctagagtggaagtgggggtgggttaacctcacacaagcacagatcccagactttgccagaggcaaacagggaattccgccgatactgacgggctccaggagtcgtcgccacactcg

SEQ ID NO: 6 - protein, 344 aa, Homo sapiens
MHRTTRIKITELNPHLMCALCGGYFIDATTIVECLHSFCKTCIVRYLETNKYCPMCDVQVHKTRPLLSIRSDKTLQDIVYKLVPGLFKDEMKRRRDFYAAYPLTEVPNGSNEDRGEVLEQEKGALSDDEIVSLSIEFYEGARDRDEKKGPLENGDGDKEKTGVRFLRCPAAMTVMHLAKFLRNKMDVPSKYKVEVLYEDEPLKEYYTLMDIAYIYPWRRNGPLPLKYRVQPACKRLTLATVPTPSEGTNTSGASECESVSDKAPSPATLPATSSSLPSPATPSHGSPSSHGPPATHPTSPTPPSTASGATTAANGGSLNCLQTPSSTSRGRKMTVNGAPVPPLT

SEQ ID NO: 7 - DNA, 1605 bp, Mus musculus
  CDS (408)..(1436)
  ref <308>: DATABASE ACCESSION NUMBER: D90085
  ref <309>: DATABASE ENTRY DATE: 2002-12-19
  ref <313>: RELEVANT RESIDUES: (1)..(1605)
gcgttccattcttctccgcccgagcagctgagcgcgccagcctcggccacagccaagccccgaggccaccgtgcgtttcccgcgaggtttctcccctcccgccgaccgagcgagcgacagggctgtggtccccctcccctcccttccctccctccctcccatccccccctccccgagacccaccggaccccgaagccagatggccgaagtggattccccctcttaacgatttggagtctccctcgaccacctcctctttgtgcagcagccccctgctaagaccctgcttgcgaggcaggaaacagcctccaggatccagtccggctgtcgcgtcgcctgatcctgttcaagttgaaggcttgggagggtgacacttcccaaatctcctcagtcctgaccccggcatcatgcatcggaccacacggattaaaatcacggagctgaaccctcacctcatgtgtgccctctgtgggggctattttattgatgccaccaccattgtggaatgcttacattccttctgcaaaacctgcatcgtacgctacttggagaccaacaaatactgccccatgtgtgacgtccaggtccataaaactcgaccactgctgagcatcagatcagacaaaaccctccaggacattgtctacaagttggtgcctgggctttttaaagatgaaatgaaacggcgacgggacttctatgcagcataccccctgacagaggtccccaacggctccaacgaggaccgaggcgaggtcctggaacaggagaagggggctctgggtgacgacgagattgtcagtctttccattgagttctacgaaggcgtcagggaccgagaggagaagaagaacctcacggagaatggagatggggacaaggagaagacaggagtgcgattcctgcggtgcccagcagccatgaccgtcatgcacctggcaaagttcctccgcaacaaaatggatgtgcccagcaagtacaaggtggagatcctctatgaagatgagcccctgaaggaatactacaccttaatggacattgcgtacatctacccctggcgaaggaatgggcctctccccctcaagtaccgtgtccagccagcctgcaagcggctcaccctgcccacggtgccaaccccctcagaaggcaccaacaccagcggggcatcagagtgtgagtcagtcagcgacaaggctcccagccctgccacccttccggccacctcctcctccttgcccagccctgcgaccccatcccatggctctcccagctcccacggccccccagctacccatcctacttcccccactccaccttcaactgccgctgggaccaccacagctaccaatgggggcacttcgaactgcctgcaaacgccatcctccaccagcagggggcgcaagatgactgttaatggcgctccttgccccccttgaggaaagggaccctctccttccgcagccatcttttccctcctttttactttttctgggcccccttttccacttttgactttccctggttcctcccattttcagagtgggaggtgggttttataaataaatctatatatatataaatatatatatgtacataggaaaaaaa

SEQ ID NO: 8 - protein, 342 aa, Mus musculus
MHRTTRIKITELNPHLMCALCGGYFIDATTIVECLHSFCKTCIVRYLETNKYCPMCDVQVHKTRPLLSIRSDKTLQDIVYKLVPGLFKDEMKRRRDFYAAYPLTEVPNGSNEDRGEVLEQEKGALGDDEIVSLSIEFYEGVRDREEKKNLTENGDGDKEKTGVRFLRCPAAMTVMHLAKFLRNKMDVPSKYKVEILYEDEPLKEYYTLMDIAYIYPWRRNGPLPLKYRVQPACKRLTLPTVPTPSEGTNTSGASECESVSDKAPSPATLPATSSSLPSPATPSHGSPSSHGPPATHPTSPTPPSTAAGTTTATNGGTSNCLQTPSSTSRGRKMTVNGAPCPP

SEQ ID NO: 9 - DNA, 4058 bp, Homo sapiens
  CDS (210)..(3224)
  ref <308>: DATABASE ACCESSION NUMBER: NM_004426
  ref <309>: DATABASE ENTRY DATE: 2003-09-06
  ref <313>: RELEVANT RESIDUES: (1)..(4058)
cccgccctcggcgcccccgcccctccagaaaggggaggaggcgaggggagcccgccgcggaggccgagcgagccggcgcccagcccagcctggcgactggggaccccggcacatgaggtggacgccccggggaagacttgggtgcacagccaggcgagaaggtcttgagtcagacagagcaccagccttggggaccctggaccactatcatggagactgagagcgagcagaactccaattccaccaatgggagttctagctcagggggcagctctcggccccagatagctcaaatgtcactttatgaacgacaagcagtgcaggctctgcaagcactgcagcggcagcccaatgcagctcagtatttccaccagttcatgctccagcagcagctcagtaatgcccagctgcatagcctggctgccgtccagcaggccacaattgctgccagtcggcaggccagctccccaaacaccagcactacacagcagcagactaccaccacccaggcctcgatcaatctggccaccacatcggccgcccagctcatcagccgatcccagagtgtgagctctcccagtgctaccaccttgacccaatctgtgctactggggaacaccacctccccacccctcaaccagtctcaggcccagatgtatctacggccacagctgggaaacctattgcaggtaaaccgaacccctgggtcgaatgtgcctctagcctcccaactcatcctgatgcctaatggggcggtggctgcagtccagcaggaggtgccatctgctcagtctcctggagttcatgcagatgcagatcaggttcagaacttggcagtaaggaatcaacaggcctcagctcaaggacctcagatgcaaggctccactcagaaggccattcctccaggagcctcccctgtctctagcctctcccaggcctctagccaggccctagcggtggcacaggcttcctctggggccacaaaccagtccctcaaccttagtcaagctggtggaggcagtgggaatagcatcccagggtccatgggtccaggtggaggtgggcaggcacatggtggtttgggtcagttgccttcctcaggaatgggtggtgggagctgtcccaggaagggtacaggagtggtgcagcccttgcctgcagcccaaacagtaactgtgagccagggcagccagacagaggcagaaagtgcagcagccaagaaggcagaagcagatgggagtggccagcagaatgtgggcatgaacctgacacggacagccacacctgcgcccagccagacacttattagctcagccacctacacacagatccagccccattcactgattcagcaacagcaacagatccacctccagcagaaacaggtggtgatccagcagcagattgccatccaccaccagcagcagttccagcaccggcagtcccagctccttcacacagctacacacctccagttggcgcagcagcagcagcagcaacaacagcaacagcagcaacagcagcagccgcaagccaccaccctcactgcccctcagccaccacaggtcccacctactcagcaggtcccaccttcccagtcccagcagcaagcccaaaccctggtcgttcagcccatgcttcagtcttcacccttgtctcttccacctgatgcagcccctaagccaccaattcccatccaatccaaaccacctgtagcacctatcaagccgcctcagttaggggccgctaagatgtcagctgcccagcaaccaccaccccatatccctgtgcaagttgtaggcactcgacagccaggtacagcccaggcacaggctttggggttggcacagctggcagctgctgtacctacttcccgggggatgccaggtacagtgcagtctggtcaggccctaaaggcttcctcgccaccttcatcccaggctcctggtgcactgcaggagtgccctcccacattggccccggggatgacccttgctcctgtgcaggggacagcacatgtggtaaagggtggggctaccacctccacacctgttgtagcccaggtccctgctgccttctatatgcagtctgtgcacttgccgggaaaaccccagacattggctgtcaaacgcaaggctgactctgaggaggagagagatgatgtctccacattgggttcaatgcttcctgccaaggcatctccagtagcagaaagcccaaaagtcatggacgagaagagcagtcttggagaaaaagctgaatcagtggctaatgtgaatgctaatactccaagcagtgaactagtagccttgacccccgccccttcagtaccgcctcctacactagccatggtgtctagacaaatgggtgactcaaaacccccacaggccatcgtgaagccccagattctcacccacatcattgaaggctttgttatccaggaaggagcagaacctttccccggtggttgttctcagttactgaaggagtctgagaagccactacagactggccttccgacaggcttgactgagaatcagtcaggtggcccctttggagtggacagcccatctgctgagttagataagaaggcgaatctcctgaagtgcgagtactgtgggaagtacgcccccgcagagcagtttcgtggctctaagaggttctgctccatgacctgcgctaagaggtacaatgtgagctgtagccatcagttccggctgaagaggaaaaaaatgaaagagtttcaagaagccaactatgctcgcgttcgcaggcgtggaccccgccgcagctcctctgacattgcccgtgccaagattcagggcaagtgccaccggggtcaagaagactctagccggggttcagataattccagttatgatgaagcactctctccaacatctcctgggcctttatcagtaagagctgggcatggagaacgtgacctggggaatcccaatacagctccacctacaccggaattacatggcatcaaccctgtgttcctgtccagtaatcccagccgttggagtgtagaggaggtgtacgagtttattgcttctctccaaggctgccaagagattgcagaggaatttcgctcacaggagattgatggacaggcctttttattacttaaagaagaacatcttatgagtgccatgaacatcaagctgggccctgccctcaagatctgtgccaagatcaatgtcctcaaggagacctaaggtggccctcttgcacaaaccagcctaaggcagacactctccactgtccaggttataacctggtaccagcagactttgcagggaagaaagagttgttccaatcatgtaaccttctgtaggggattactgagacagggaagagaagtgcaagaattggttgctggtgctacatggcggcagctttgacattttctctgggttctactttattttttaaaatctttacagttctcaccatttcacgtaccttaatccaatctttataaaagaggcagtctagagaactaggactgctcagccttatcctggagtggagcatttagcccaggtcttaattctccaagaggaggaatacatagtatggtaaggcaaggaactgggtggaatgtcaggttgcctgcccaatgggagaggtagggtttttctagcttgtgtgacagaagtagcaaaatctggtcctcccccctcccagtgtagctgtggctcagagttttttctttttgttgtcacttactcccttgtgattgaattttttctcctgcatccatggcaggatccccagccagtatagagacttggttggcatcttctgctgcagggactaaaagtatttgactggggcacatgtggctgttgtcattctttctgcatcccactgttcccctccaatttatgttattttctaccctgtttttcagttccatctctgctctgtcctatagctttataaaaccagagtgtgtggggctgaggtcaggagtataagtacctgccttaggcactattccttatataacaaaaatattaaatatttttttcctcagtaaaaggatg

SEQ ID NO: 10 - protein, 1004 aa, Homo sapiens
METESEQNSNSTNGSSSSGGSSRPQIAQMSLYERQAVQALQALQRQPNAAQYFHQFMLQQQLSNAQLHSLAAVQQATIAASRQASSPNTSTTQQQTTTTQASINLATTSAAQLISRSQSVSSPSATTLTQSVLLGNTTSPPLNQSQAQMYLRPQLGNLLQVNRTPGSNVPLASQLILMPNGAVAAVQQEVPSAQSPGVHADADQVQNLAVRNQQASAQGPQMQGSTQKAIPPGASPVSSLSQASSQALAVAQASSGATNQSLNLSQAGGGSGNSIPGSMGPGGGGQAHGGLGQLPSSGMGGGSCPRKGTGVVQPLPAAQTVTVSQGSQTEAESAAAKKAEADGSGQQNVGMNLTRTATPAPSQTLISSATYTQIQPHSLIQQQQQIHLQQKQVVIQQQIAIHHQQQFQHRQSQLLHTATHLQLAQQQQQQQQQQQQQQQPQATTLTAPQPPQVPPTQQVPPSQSQQQAQTLVVQPMLQSSPLSLPPDAAPKPPIPIQSKPPVAPIKPPQLGAAKMSAAQQPPPHIPVQVVGTRQPGTAQAQALGLAQLAAAVPTSRGMPGTVQSGQALKASSPPSSQAPGALQECPPTLAPGMTLAPVQGTAHVVKGGATTSTPVVAQVPAAFYMQSVHLPGKPQTLAVKRKADSEEERDDVSTLGSMLPAKASPVAESPKVMDEKSSLGEKAESVANVNANTPSSELVALTPAPSVPPPTLAMVSRQMGDSKPPQAIVKPQILTHIIEGFVIQEGAEPFPGGCSQLLKESEKPLQTGLPTGLTENQSGGPFGVDSPSAELDKKANLLKCEYCGKYAPAEQFRGSKRFCSMTCAKRYNVSCSHQFRLKRKKMKEFQEANYARVRRRGPRRSSSDIARAKIQGKCHRGQEDSSRGSDNSSYDEALSPTSPGPLSVRAGHGERDLGNPNTAPPTPELHGINPVFLSSNPSRWSVEEVYEFIASLQGCQEIAEEFRSQEIDGQAFLLLKEEHLMSAMNIKLGPALKICAKINVLKET

SEQ ID NO: 11 - DNA, 3851 bp, Mus musculus
  CDS (27)..(3065)
  ref <308>: DATABASE ACCESSION NUMBER: U63386
  ref <309>: DATABASE ENTRY DATE: 1998-08-07
  ref <313>: RELEVANT RESIDUES: (1)..(3851)
attcttggggaccctggaacgctatcatggaaacggagagtgagcagaactctagttcgaccaatgggagttccagttcaggggccagctctcggccccagatagcacagatgtccctgtatgaacggcaagcagtgcaggctctgcaagcactgcagcggcaacctaatgcggctcaatatttccaccagttcatgctccagcaacagctcagcaatgcccaactgcacagcctggcagctgtccagcaggccacgattgctgccagtcgacaggctagctccccaaacagcagcactgcacagcagcagaccgctaccacccaggcctcgatgaatctcgcgaccacgtcagctgctcagctcatcagccgatcgcagagtgtgagctcccccagtgctaccgccctgacgcagtgggtgctgctggggaacaccacttccccacctctcaaccagtcccaggcccagatgtatctgcggccacagctgggcaacctattgcaggttaaccggaccttgggccggaatgtgcctcttgcctcccagctcatcctgatgcctaatggggcagtggctgcagtccagcaggaggtgccacccgctcagtctcctggagttcatgcagatgcagatcaggtgcagaacttggcagtgaggaaccaacaggcttcagctcaaggaccccaaatgccaggctccactcagaaggccatccctcctggagcttctcctgtttctggcctctcccagacttctagccaggccctagccgtggcacaggcttcttctggggcctcaggccagtcactcaaccttagtcaagccggtggaggcagtgggaatagcctcccaggctccatgggtccaggtggaggtggccaggcgcctgggggtttaggtcagttgccttcttcaggattgactggtggaagctgtccccggaagggcacaggagtggtgcagcccttacctgcagctcaaacagtgactgtgagccaaggaagccaaacagaagcagaaagtgcagcagccaagaaggcagaagcagatgggagtggccagcagagtgtgggcatgaacctgactcggacagccacacctgcccccagccagacgcttattagctcagccacatacacacagatccagccccattcactgattcagcaacagcagcagatccacctccagcagaagcaagtggtgatccagcagcagattgccatccaccaccagcaacagttccaacaccgccagtcccagctgcttcacacagccacacacctccagttggcccagcagcagcagcagcaacagcagcaacagcagcaacaacagcaacagcagcagcagcagcagcagcagcaaggaacaaccctcactgcccctcagccaccccaggtcccacctactcagcaggtcccaccttcccaatcacaacagcaagcccagactctggtggtccaacccatgcttcagtcttcacccctgactcttccacctgaaccaacctctaaaccacccatccccattcagtccaaacctcctgtagcgcctatcaaacctcctcaactaggagctgctaagatgtcagctacacagcagccaccaccacatatccccgtgcaagttgtcggtacccggcagccaggttcagcccaagcacaggctttgggattagcacagctggctgctgctgtacctactccccgtggaataacaggagcagtccagcctggccaggctcatctggcctcctcgccaccttcatcacaggcggctcctggtgcacttcaagaatgtcctcctgcattggctgctgggatgacccttgctcctgtgcaggggacagcacatgtggtaaagggtgggcctacagcctcctctcctgttgtggcccaggtccctgctgctttctacatgcagtcggtgcacctgccgggtaaagcccagaccttggctgtgaaacgcaaagctgagtctgaggaggagagagatgacctctccgcattggcctctgtgcttcctacaaaggcctccccagcagcagagagcccaaaggtaatagaggagaagaatagtcttggagagaaagctgaaccagtggccagtttgaatgctaatcctccaaacagtgatctagtagccttggcccctaccccatcagcaccacctcctactctggccttggtgtccagacagatgggtgactcaaaacccccacaggccattgtgaagccccagattctcacacacatcattgaaggctttgttatccaggaaggagcagagccttttccggtgggatgttctcagttcctgaaagaaaccgagaagccactgcaggctggccttccgaccgggctgaatgagagtcagccaagtggccctttggggggagacagcccctctgtagagttagagaagaaggcaaatctcctcaagtgtgagtactgtggcaagtacgccccagcagagcagtttcgaggctctaagaggttctgctccatgacgtgtgcaaagaggtacaatgtgagctgcagccaccagttccgcttgaagaggaaaaaaatgaaagagtttcaggaagccagctatgcacgtgttcggaggcgtggaccccgccgcagctcctctgatattgctcgtgctaagatccagggcaagcgccatcggggtcaagaggactctagccggggatcagataattccagctatgatgaagcactctctccaacatctcctgggccgctgtcagtaagagccgggcacggagaacgtgatctaggaaacaccattacaaccccatccacgccagagttacagggcatcaatcccgtgttcttgtccagtaatcctagccagtggagcgtcgaggaggtctacgagtttattgcttctctgcaaggctgccaagagattgcagaagagtttcgttcccaggagattgatggacaggcccttttattacttaaagaagaacatctgatgagtgccatgaacatcaaattaggtcctgctctcaagatctgtgccaagatcaatgttctcaaggagacctaagtgaccctctgcacaattcagactaaggcagaaggcctggctgttcaggttataacctggaagagctgttccttttgtgtgacctgtggggtttggaggcagaactcttaaggattggttgctggtgctacatggtgacagctttgataccttctctgggtttcactttaacaataaaaaccttctcactattcacacctaccctaatccagtttttaaacaggcagactagagactaggactgcttagccttccttatcctggagtggagggactagccagggttcacttctccaaggaggaatggatggtaaggcaaggaaatggatgccatgtaggtttgcctccctatgggagaggtcagctttcctagcttatgacaggagcagcaaacctggtactctgctggagtctcactgtggctgtagctcagttttctgtttcattttctcccttaatactgaatatttcttctgcattcatggcagggtcaccagccatcatagacacttggttggtatcggttttgctgcagggacgaagtgtttgagtgacacatgtggctgttgccattctttctgcaccccagctcccaactgttcccaccttcaaccctctttcccattccaacgctgctctgtcctatagcttcacaaaacaggagcgtgtggggctgaggtcaggactgtaccttgggcactattccttatacaaaatattaaatattttttttcctcagtaaaaggatgaaagttggcctccaaaaaaaaaaaaaaaaaaaaaaaa

SEQ ID NO: 12 - protein, 1012 aa, Mus musculus
METESEQNSSSTNGSSSSGASSRPQIAQMSLYERQAVQALQALQRQPNAAQYFHQFMLQQQLSNAQLHSLAAVQQATIAASRQASSPNSSTAQQQTATTQASMNLATTSAAQLISRSQSVSSPSATALTQWVLLGNTTSPPLNQSQAQMYLRPQLGNLLQVNRTLGRNVPLASQLILMPNGAVAAVQQEVPPAQSPGVHADADQVQNLAVRNQQASAQGPQMPGSTQKAIPPGASPVSGLSQTSSQALAVAQASSGASGQSLNLSQAGGGSGNSLPGSMGPGGGGQAPGGLGQLPSSGLTGGSCPRKGTGVVQPLPAAQTVTVSQGSQTEAESAAAKKAEADGSGQQSVGMNLTRTATPAPSQTLISSATYTQIQPHSLIQQQQQIHLQQKQVVIQQQIAIHHQQQFQHRQSQLLHTATHLQLAQQQQQQQQQQQQQQQQQQQQQQQQGTTLTAPQPPQVPPTQQVPPSQSQQQAQTLVVQPMLQSSPLTLPPEPTSKPPIPIQSKPPVAPIKPPQLGAAKMSATQQPPPHIPVQVVGTRQPGSAQAQALGLAQLAAAVPTPRGITGAVQPGQAHLASSPPSSQAAPGALQECPPALAAGMTLAPVQGTAHVVKGGPTASSPVVAQVPAAFYMQSVHLPGKAQTLAVKRKAESEEERDDLSALASVLPTKASPAAESPKVIEEKNSLGEKAEPVASLNANPPNSDLVALAPTPSAPPPTLALVSRQMGDSKPPQAIVKPQILTHIIEGFVIQEGAEPFPVGCSQFLKETEKPLQAGLPTGLNESQPSGPLGGDSPSVELEKKANLLKCEYCGKYAPAEQFRGSKRFCSMTCAKRYNVSCSHQFRLKRKKMKEFQEASYARVRRRGPRRSSSDIARAKIQGKRHRGQEDSSRGSDNSSYDEALSPTSPGPLSVRAGHGERDLGNTITTPSTPELQGINPVFLSSNPSQWSVEEVYEFIASLQGCQEIAEEFRSQEIDGQALLLLKEEHLMSAMNIKLGPALKICAKINVLKET

SEQ ID NO: 13 - DNA, 4244 bp, Homo sapiens
  CDS (386)..(1303)
  ref <308>: DATABASE ACCESSION NUMBER: XM_300674
  ref <309>: DATABASE ENTRY DATE: 2003-04-23
  ref <313>: RELEVANT RESIDUES: (1)..(4244)
tgctccctccaaatccaagtccagcagttcctcctcttcctccacgtcatcctcctcttcctcagatgaagaggatgacagtgacttagatgctaagaggggtccccggggccgcgagacccacccagtgccgcagaagaaggcccagatcctggtggccaaacccgagctgaaggatcccatccggaagaagcggggacgaaagcccctgcccccagagcaaaaggcaacccgaagacccgtgagcctggccaaggtgctgaagaccgcccggaaggatctgggggccccggccagcaagctgccccctccactcagcgcccccgttgcaggcctggcagctctgaaggcccacgccaaggaggcctgtggcggccccagtgccatggccaccccagagaacctggccagcctaatgaagggcatggccagtagccccggccggggtggcatcagctggcagagctccatcgtgcactacatgaaccggatgacccagagccaggcccaggctgccagcaggttggcgctgaaggcccaggccaccaacaagtgcggcctcgggctggacctgaaggtgaggacgcagaaaggggagctgggaatgagccctccaggaagcaaaatcccgaaggcccccagcggtggggctgtggagcagaaagtggggaacacagggggccccccgcacacccatggtgccagcagggtgcctgctgggtgcccaggcccccagccagcacccacccaggagctgagcctccaggtcttggacttgcagagtgtcaagaatggcatgcccggggtgggtctccttgcccgccacgccaccgccaccaagggtgtcccggccaccaacccagcccctgggaagggcactgggagtggcctcattggggccagcggggccaccatgcccaccgacacaagcaaaagtgagaagctggcttccagagcagtggcgccacccacccctgccagcaagagggactgtgtcaagggcagtgctacccccagtgggcaggagagccgcacagcccccggagaagcccgcaaggcggccacactgccagagatgagcgcaggtgaggagagtagcagctcggactccgaccccgactccgcctcgccgcccagcactggacagaacccgtcagtgtccgttcagaccagccaggactggaagcccacccgcagcctcatcgagcacgtatttgtcaccgacgtcactgccaacctcatcaccgtcacagtgaaggagtctcccaccagcgtgggcttcttcaacctgaggcattactgaagccccggcgccaccagctgcgcggtcttactccccttccctgcctatggtgtcgcttggctaagtgactcccagcccaagccccctcaagagtctgggtcgggggaggaggagtgggtggcctccttgatgggcaggcttggaagggacttctcccgcaccccactctgtcccaggacatagggcagggggcctcactgccttgttggtctccaccttgttcctacctctgcaggcctctttgctctcccctcttgcctcaggaaacccggtggcacctgtggctccaggtgactgtcttgaacagagcgggcttcttcatggctgcgttgttgctgagtttgaactgctcctccctggcctgcgtgactgaatcacagctttggtccctgtcttgcaggggctgaggtgtcaggaggggacttctggcccaccttgccttcagccctggagtgggcagagagtattgtggggaggcatggccagtgggactagtgttccctccatctggccacagcttttgggagatggggtgggcaggggtggtcctggctggcattgcctgagccggcagtgatgaagtggggagcttgcccttgacaggtgggggctggctggggccttaatgtgaaaagacagtggcaggcagctggagtagagcgagcccagcagccctaaaaggctgccttcatggccatctagccccagttcagggcagcatccatagcccacaagccagcgtgggtggggcgggggtggtcccacagctgggttccacctgaagagcctccgtgcctcggagcaggagaggcaggctatggctgccaccctccctcctgcctgtgtcccagtgagaactgacctgagtccccttccaaacccagacccacctcctgccccaggcccactgaagcatgttccatttctaaaaagcccagagttcagtgtgtcccaaggaaaacccaaagtggaggtgctcaggtccaggggagtccagtgggcaggacccttggcaggcaagcccctcccttcactcccaggacctaccttctgctagtaaaggactggcttcattctaattatggcccacagactgccccggagacctggaggacagcagtgctggcacttgggtgtccatgggcccgtctgccggctctgcctgtgctgcaagtgttggccgtgggtccagccaacaactccctacgtcctgtgtggggccctgcccaagtggatgaggcattccttgaggagtatcattttccctgacaatccccatcacctttaggggttccctgcttggctcctttccagctgaaaaactagacctgtgccattggggaagctggacaaagtctagggggcccgcctggtagagggtcccgggaagctggatctgtcagcctcggccctgaggcccctgttaactcaagactgtgagctgcctctaggtggtcacgtctgggagctagcttgtatggcttctgaccagtatcaggatttctgttctgagagcagcgtgggcagcaaggcagggcagcccagaggtggcagcggcaggcaatctggtcactaggtctttgtgatgccaaaaataaaagagggtggggtgggtgctttctgttcctctgattggatggagtccgccagcaggcatggggctacattccagtgcctgactatagggaggcactcctgattccatggagcagcccggactttgagaatgggctctggtttgcggggggcaggcgtaccagactgcaagaccccccagtacctcaccgtgccaaataggaagaggtggccttggtgtagccaaatggatctttttaacagtgtgcctttggggagggacccatgtccatggcttcgttgagggccatccatatgccagctgggggccagcccacagtggccatattggctgcagcaggaatggtgcccacctcggcgaattgaagggctaagagtcccagatagctaggccagagctggaagcagacagtaaggggaagagctgctcccacaggagagggagagattccagctcactgcgcagcctgggaggaggcgtggatcctggcacgctgagcctcaggcaccagcctccctgtgctcgacagcaaagtcttgactccttcctgctgagcactgtgctaccttcactgctccaaagccagactaacagctctccaagcccttggggtgactcggcttccaggagctgttggagaaatgaggatgtctgtccctgtctgcctgggcaggccagattcctccccagcagccgggtctctccagaccctgattcggtgcctttctgtttaccagctacttcaatcccaaagtttgaatctgcagataccttactcccagccactttgccttcttactgtgttgtgtgtttttcctggtgcttcaagagcgtgtgcagggcaagtgccgtcactgggaactgcaccagatgctcagacttggttgtcttatgtttaccaataaataaaagtagactttttctatttttatttgctgctatttgtgtgtgtgtttgtgtttgtgtagctaggtatctggcacttctgacgatgcattgttgcttttttcccgaaggtcccgcaggaactgtggcaatggtgtgtgtgtgaaatggtgtgttaaccgcgttttgtttgctcctgtattgaataggaagcagtggccagtctgtcttccttagagatgttagcatatttttatatgtatatattttgtaccaaaaaagagtgttccttgttttggttacactcgaaattctgacctagctggagagggctctgggccgagagctttcactaaggggagacttcaggggaggatcaagctttgaaccaaagccaatcactggcttgatttgtgttttttaattaaaaaaaaaatcattcatgtatgccacttctaa

SEQ ID NO: 14 - protein, 305 aa, Homo sapiens [sequence omitted]

SEQ ID NO: 15 - DNA, 3754 bp, Mus musculus
  CDS (81)..(1640)
  ref <308>: DATABASE ACCESSION NUMBER: BC035199
  ref <309>: DATABASE ENTRY DATE: 2003-04-16
  ref <313>: RELEVANT RESIDUES: (1)..(3754)
ggcggggcgcggcgggggcggcgctttgtgtgcagcagtgagccggggtctgcggggcgcgggccgcggcgggcggcggcatggaggagctgagcagcgtgggcgagcaggtcttcgccgccgagtgcatcctgagcaagcggctccgcaagggcaagctggagtacctggtcaagtggcgcggctggtcctccaaacacaacagctgggagccagaagagaacattttggacccgaggctgctcctagccttccagaagaaggaacatgagaaggaggttcagaaccggaagagaggcaagagacccaggggcaggccgaggaaacacacagtcacatcctcctgcagccggcgctccaagctcaaggaaccagatgcgccatccaaatccaaatccagcagttcgtcctcttcctccacatcttcctcctcttcctcggacgaagaggaagacgacagcgacctagactccaagagaggcccccggggccgtgaaacccatccagtgcctcagaaaaaagcccagatcctggtagccaagccagagctgaaggatcccattagaaagaaacggggacgcaagcctctacccccagaacagaaggcagctcggagacccgtcagcctggccaaggtgctaaagaccaccaggaaggatctggggacctcagccgccaagctgccccctccactcagcgctccggtggcaggcctggctgccctgaaggcccacaccaaagaggcctgtggtggccccagcactatggcgaccccagagaacctggccagtctgatgaaaggcatggccgggagccccagcagaggcggcatctggcagagctccatcgtacactacatgaaccgcatgagccagagtcaggttcaggctgccagccgactggcactcaaggcccaggccaccaacaagtgcggtctcgggctagacctgaaagtgaggacgcagaaggggggtgagctaggggggagccccgcaggaggcaaggtcccgaaggcccccggtggcggagctgcagagcagcagagagggaaccattcggggagcccaggtgctcagctggcacccactcaggagttgagccttcaggtccttgacttgcaaagcgtcaagaacggtgtgcctggtgtgggcctgcttgctcgccatgccccagccaaggctattcctgctaccaacccagccacagggaaaggtcctgggagcggccccacaggagcaaacatgaccaacgctcccacagacaacaacaaaggggaaaagctgacttgcaaggcaacggctctgcctgccccttccgtcaagcgggacaccgttaaaagcgtcgctgcctccggcgggcaggagggccacacagccccgggagaaggccgaaagccacctgcgctgtctgagctgagcacgggagaggagaatagtagctctgactcggaccctgactcgacctcgcttcccagtgctgcgcagaacctatctgtagctatccagaccagccaggactggaaacctacccgcagtctcatcgaacacgtctttgtcacggatgtcacagccaacctcatcactgtcaccgtgaaggagtcgcccaccagcgtgggcttcttcaacttgaggcattattgagaccattgcccgagccagacctgccttccagtctctggttttgctttgatgcctgacctgggggccaactctgccctgtcctgagactcagggctgggaaatggcttcctgctgattctacctctctgggcctccacttcagtctgtcctgtcatctcaggaaagccgggtggccactgacttaaaagctacctgcttggacatagcatcctttctacacatccctccttaacctagtgagaccctcccctccctggccaactactgtgaggtggattggggaggggggggcactgatacagctccctccccaggccgttgaggagaggggtggggctggtagggccttctcccacatccagccacggcagggtctttctggtgagggagacactctatgctttgcaagtaaggctataacttcagttttaaaaggctaattagttgtgtggttgggctcagggaccctgcggaaatcttcagtctctatccgtggtgtggtgtcccctctagttgcaattgtagtcccaaagcccagtcagggtttggagtcctggcactaaggtgtacctgggtgcttctggacttgggaatagagagagccatgtccttattgcctccttcaagcaggctcaagccccagtgagaagtgaccctgttcctctctgggtctggacccatacccctgctttggcccactaaattgcaccctagttctgaggtctgacactcctgggcttggggaaggagcaggctagacacccagagcaagctgatgtaggcacttggagtcctgggcaagcaatcttccttcatgttggacttttattactaggaccccataagtcaccatggtggcaccagtgtgggaatgcaggtgttttgtgggggcctgtcaccctgctgcctgtctagacatctctaaactactgtggaagcattttctatagtcagtcttggtgcagagctccgcctggtgagtgggccactcttgggtcccatattccctgacaatcctctcccacatttgggagcttgtgtttcctccctcgctgagaagcaatagccctatgccatttgtgtggtggggtcgtttaggagcccacctggcagggggcccctaatttctttcccatcctaggagataagtctagtgctgagcccctttctaaatgccttctcccctgtcccccatcccgggtaactgtcttgaaactaccttgtcagattgctggccgatgacaggattttctgtcctgggatatcacggtgggccgtgacaatctggtcagtgagtctgtgatgccaaaaagtaggggtgctatgaaaccctggttctgcttctgccattgactgatcagccagcaggtgtgggctcttcactccagttgccttgaagggaaatgtttccccttctaaaaccagtcagacagtgggctgaggtgtctgggacctgccacactttcaaagtgcaccccacccccaccccagtagctcaccctgcacaccaaataagatggtctttgtgtaaccaaaccaacctcaatgctgtgccttttggggcagacccatatccacagctcctgagagatgcctgtgatgtgccaggggaccatgttactggcagcaggagctacgtactgggaagaattcagggccaggaagtgggtaacaaaggggaaagggttgcctactgcattatcaatgtccctccctgccctcagccagggtctgcaacctttgtaccagacactgcctttacaaacacctccccgtctctttggggtcccatgagctgttggagaagtgaccctgtttggggatctgggttgactggcctttgtcccctgctgccagtctccaaatcccaatttggtgccttttattttactggttacgtcagtcccaaagttgaaatctacaaatcctttcctctcagccactttgccttcttgtgtgtttttcctggtgctgtgaccccagtggtctatgtcacaaagcacatgtgtgtagtgcgccagcgcgagccccactggagcagcgccattcgctctgagtcaagttctcgtgtttaccgataaataaagtagttggtttttaaaaaaaaaaaaaaa

SEQ ID NO: 16 - protein, 519 aa, Mus musculus
MEELSSVGEQVFAAECILSKRLRKGKLEYLVKWRGWSSKHNSWEPEENILDPRLLLAFQKKEHEKEVQNRKRGKRPRGRPRKHTVTSSCSRRSKLKEPDAPSKSKSSSSSSSSTSSSSSSDEEEDDSDLDSKRGPRGRETHPVPQKKAQILVAKPELKDPIRKKRGRKPLPPEQKAARRPVSLAKVLKTTRKDLGTSAAKLPPPLSAPVAGLAALKAHTKEACGGPSTMATPENLASLMKGMAGSPSRGGIWQSSIVHYMNRMSQSQVQAASRLALKAQATNKCGLGLDLKVRTQKGGELGGSPAGGKVPKAPGGGAAEQQRGNHSGSPGAQLAPTQELSLQVLDLQSVKNGVPGVGLLARHAPAKAIPATNPATGKGPGSGPTGANMTNAPTDNNKGEKLTCKATALPAPSVKRDTVKSVAASGGQEGHTAPGEGRKPPALSELSTGEENSSSDSDPDSTSLPSAAQNLSVAIQTSQDWKPTRSLIEHVFVTDVTANLITVTVKESPTSVGFFNLRHY

SEQ ID NO: 17 - DNA, 4808 bp, artificial sequences
  Cloning vector pGIU2
  CDS (65)..(781)
  ref <308>: DATABASE ACCESSION NUMBER: AJ249646
  ref <309>: DATABASE ENTRY DATE: 2001-07-02
  ref <313>: RELEVANT RESIDUES: (1)..(4808)
gaattcgagctcggtacccggggatcctctagagtcgacctgcaggcatgcaagctttattaaaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactgcagcccctgagcgccggtcgctaccattaccagttggtctggtgtcaaaaataataataaccgggcaggccatgtctgcccgtatttcgcgtaaggaaatccattatgtactatttcgcctgatgcggtattttctccttacgcatctgtgcggtatttcacaccgcatagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaaaagctcttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttattttagtagctcgttacagtccggtgcgtttttggttttttgaaagtgcgtcttcagagcgcttttggttttcaaaagcgctctgaagttcctatactttctagctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattcccg

SEQ ID NO: 18 - protein, 238 aa, artificial sequences
  Cloning vector pGIU2
MSKGEELFTGVVPILVELDGDVNGHKFSVSGEGEGDATYGKLTLKFICTTGKLPVPWPTLVTTFGYGVQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 19 - DNA, 855 bp, Mus musculus
  CDS (1)..(855)
atgcatcgaacaaccagaatcaagatcactgagctaaatccccacttaatggatgtccaggttcacaaaaccagaccactcctgaacataaggtcagataaaactcttcaagatattgtatacaaattagtcccagggcttttcaaaaatgagatgaagagaagaagagatttttatgcagctcacccgtcagctgatgctgccaatggctccaatgaagaccgaggagaagttgcagatgaggagaagaggattataactgatgatgagataataagcttgtctattgagttctttgatcagagcagattggatcggaaagtaaataaagagaagcctaaggaagaggtgaatgataaaaggtacttacgatgcccagcagcaatgactgtgatgcacttgagaaagtttctcaggagtaaaatggacatacccaatactttccagattgacgtcatgtatgaagaggaacctttaaaggattactacacgctaatggacattgcctacatttatacctggagaagaaatggcccactacctttgaaatacagagttcggccaacttgcaaaagaatgaagatgagtcaccagagggatggactgacgaatgctggagagctggaaagtgactctgggagtgacaaggccaacagcccagccggaggtgttccctccacctcttcctgtttgcccagtcccagcactccagtgcagtctcctcacccacagttccctcacatttccagtaccatgaatggaaccagcaacagccccagtgctaaccaccaatcttcctttgccagtagacctcgaaaatcatcactaaatgggtcatcagcaacttcatctggt

SEQ ID NO: 20 - protein, 285 aa, Mus musculus
MHRTTRIKITELNPHLMDVQVHKTRPLLNIRSDKTLQDIVYKLVPGLFKNEMKRRRDFYAAHPSADAANGSNEDRGEVADEEKRIITDDEIISLSIEFFDQSRLDRKVNKEKPKEEVNDKRYLRCPAAMTVMHLRKFLRSKMDIPNTFQIDVMYEEEPLKDYYTLMDIAYIYTWRRNGPLPLKYRVRPTCKRMKMSHQRDGLTNAGELESDSGSDKANSPAGGVPSTSSCLPSPSTPVQSPHPQFPHISSTMNGTSNSPSANHQSSFASRPRKSSLNGSSATSSG

SEQ ID NO: 21 - DNA, 804 bp, Mus musculus
  CDS (1)..(804)
atgcatcgaacaaccagaatcaagatcactgagctaaatccccacttaatgtgtgtcctgtgtggagggtacttcattgatgccactaccataatagaatgtctacattccttctgtaaaacatgtattgtacgttacttggagaccagcaagtattgtcctatttgtgatgtccaggttcacaaaaccagaccactcctgaacataaggtcagataaaactcttcaagatattgtatacaaattagtcccagggcttttcaaaaatgagatgaagagaagaagagatttttatgcagctcacccgtcagctgatgctgccaatggctccaatgaagaccgaggagaagttgcagatgaggagaagaggattataactgatgatgagataataagcttgtctattgagttctttgatcagagcagattggatcggaaagtaaataaagagaagcctaaggaagaggtgaatgataaaaggtacttacgatgcttgaaatacagagttcggccaacttgcaaaagaatgaagatgagtcaccagagggatggactgacgaatgctggagagctggaaagtgactctgggagtgacaaggccaacagcccagccggaggtgttccctccacctcttcctgtttgcccagtcccagcactccagtgcagtctcctcacccacagttccctcacatttccagtaccatgaatggaaccagcaacagccccagtgctaaccaccaatcttcctttgccagtagacctcgaaaatcatcactaaatgggtcatcagcaacttcatctggt

SEQ ID NO: 22 - protein, 268 aa, Mus musculus
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEEKRIITDDEIISLSIEFFDQSRLDRKVNKEKPKEEVNDKRYLRCLKYRVRPTCKRMKMSHQRDGLTNAGELESDSGSDKANSPAGGVPSTSSCLPSPSTPVQSPHPQFPHISSTMNGTSNSPSANHQSSFASRPRKSSLNGSSATSSG

SEQ ID NO: 23 - DNA, 741 bp, Mus musculus
  CDS (1)..(741)
atgcatcgaacaaccagaatcaagatcactgagctaaatccccacttaatgtgtgtcctgtgtggagggtacttcattgatgccactaccataatagaatgtctacattccttctgtaaaacatgtattgtacgttacttggagaccagcaagtattgtcctatttgtgatgtccaggttcacaaaaccagaccactcctgaacataaggtcagataaaactcttcaagatattgtatacaaattagtcccagggcttttcaaaaatgagatgaagagaagaagagatttttatgcagctcacccgtcagctgatgctgccaatggctccaatgaagaccgaggagaagttgcagatgaggagaagaggattataactgatgatgagataataagcttgtctattgagttctttgatcagagcagattggatcggaaagtaaataaagagaagcctaaggaagaggtgaatgataaaaggtacttacgatgcccagcagcaatgactgtgatgcacttgagaaagtttctcaggagtaaaatggacatacccaatactttccagattgacgtcatgtatgaagaggaacctttaaaggattactacacgctaatggacattgcctacatttatacctggagaagaaatggcccactacctttgaaatacagagttcggccaacttgcaaaagaatgaagatgagtcaccagagggatggactgacgaatgctggagagctg

SEQ ID NO: 24 - protein, 247 aa, Mus musculus
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEEKRIITDDEIISLSIEFFDQSRLDRKVNKEKPKEEVNDKRYLRCPAAMTVMHLRKFLRSKMDIPNTFQIDVMYEEEPLKDYYTLMDIAYIYTWRRNGPLPLKYRVRPTCKRMKMSHQRDGLTNAGEL

SEQ ID NO: 25 - DNA, 1749 bp, Mus musculus
  CDS (629)..(1411)
  ref <308>: DATABASE ACCESSION NUMBER: NM_008272
  ref <309>: DATABASE ENTRY DATE: 2003-09-07
  ref <313>: RELEVANT RESIDUES: (1)..(1749)
cgcttcctcagtctgggctccaaagtcactgcaaatttccttgcgatacatacatcacacaaaagatcagagactgcaggagcgtcggagccgacagagacagattacgtcaagaaatagttcctccacctacctcgtccggcctccccgcccctgaggggctccccgagcccatctcagctctgggggtccaggaaccctggcttctgggactggcattttcatcttgtttttttttttttttaatcctcatggtagggggagccatctgcttttggctgcacatggtcaagggggggggtccctgatgagatatgattcgtccggggagggggggctgtctggcgccttgggtccagagttgcaaatcgccaggaaaacgcaggtcgcggggatcagcaaagagaagggggcggtaaaggggggaaaaagcaagggggaaaaaaagcccctgcgcattgatccgcgccgtatttttgggtaaatacgatcacgtgggggctggggaaccaatgagctgccgggaaaaggctggaaaaataattacctgccttgattgttctgtgagcagataaaaagtacatatacagttcatacaataatcttatgtatgtaaaaccctgttacgatgtcggcgacggggcccatcagtaactattacgtggactcgctcatctctcacgacaatgaagacctcctagcgtccaggtttccggccaccggggctcaccctgccgccgccagacccagcggcttggtgccggactgtagcgattttccgtcctgtagcttcgcgcccaagccggctgtattcagtacgtcgtgggcgccggtgccctcgcagtcgtctgtggtctatcacccttacggcccccagccccacctcggcgccgacacgcgctacatgcggacttggctcgagccgctgtccggcgccgtctccttccccagcttcccggccgggggccgtcactacgccctcaagcccgacgcctacccggggcgccgcgccgactgcggcccgggcgacggccgcagctacccggactacatgtacggctcgcccggggaactgcgcgaccgcgccccgcagacgctgccctcgcccgaggcggacgcgctggccggcagcaagcacaaagaggagaaggccgacctggaccctagcaaccccgtggccaactggatccacgcccgttccacaaggaagaagcgctgcccctacaccaagtaccagacgctggaactggagaaggagtttctcttcaatatgtatttaaccagggaccgtcggtacgaggtggcccgtgttctcaatctcactgagcggcaggtcaaaatctggtttcagaaccggaggatgaagatgaaaaagatgaataaagagaaaaccgacaaggaacaatcctaagccctgccccagcctgctgcctcggcacagccaagggaaacacaaaaacccccccaaaaaatgccccaacccaggcgggagaaagcccgaaaagaaaaggaaagaacaagatagagaaaagccccccgtcttaaaaagaaaagaaaaaaaaggaaggggaaaaatgcaaactcttgcgatgtgggagggttaagtgttgagaaattggtgtttagagttagttctatccatcgaggaggaggcaggagagaaactgcgtttttttccccagcgcaactgaaataaatgacccccccaaatgtgaaaaaaaaaaaaaaaaaaaaaaaaaaaaaaaaaaa

SEQ ID NO: 26 - protein, 260 aa, Mus musculus
MSATGPISNYYVDSLISHDNEDLLASRFPATGAHPAAARPSGLVPDCSDFPSCSFAPKPAVFSTSWAPVPSQSSVVYHPYGPQPHLGADTRYMRTWLEPLSGAVSFPSFPAGGRHYALKPDAYPGRRADCGPGDGRSYPDYMYGSPGELRDRAPQTLPSPEADALAGSKHKEEKADLDPSNPVANWIHARSTRKKRCPYTKYQTLELEKEFLFNMYLTRDRRYEVARVLNLTERQVKIWFQNRRMKMKKMNKEKTDKEQS

SEQ ID NO: 27 - DNA, 849 bp, Mus musculus
  CDS (82)..(588)
actggtcacacgactgggcgattgggcgggcactgaatctccgcgaggaaagcgaactcgaggagagccatctggagcagcatggagtccgctgcagacagactggccagggcggcggcccagggccgtgtgcatgacgtgcgggcactgctggaagccggggtttcgcccaacgccccgaactctttcggtcgtaccccgattcaggtgatgatgatgggcaacgttcacgtagcagctcttctgctcaactacggtgcagattcgaactgcgaggaccccactaccttctcccgcccggtgcacgacgcagcgcgggaaggcttcctggacacgctggtggtgctgcacgggtcaggggctcggctggatgtgcgcgatgcctggggtcgcctgccgctcgacttggcccaagagcggggacatcaagacatcgtgcgatatttgcgttccgctgggtgctctttgtgttccgctgggtggtctttgtgtaccgctgggaacgtcgcccagaccgacgggcatagcttcagctcaagcacgcccagggccctggaacttcgcggccaatcccaagagcagagctaaatccgcctcagcccgcctttttcttcttagcttcacttctagcgatgctagcgtgtctagcatgtggctttaaaaaatacataataatgctttttttgcaatcacgggagggagcagagggagggagcagaaggagggagggagggagggagggacttcgacaggaaaggaattgcatgagaaactgagcgaaggcggcggcgaagggaataatggctggattgtttaaaaaaataaaataaagatactttttaaaatgtc

SEQ ID NO: 28 - protein, 168 aa, Mus musculus
MESAADRLARAAAQGRVHDVRALLEAGVSPNAPNSFGRTPIQVMMMGNVHVAALLLNYGADSNCEDPTTFSRPVHDAAREGFLDTLVVLHGSGARLDVRDAWGRLPLDLAQERGHQDIVRYLRSAGCSLCSAGWSLCTAGNVAQTDGHSFSSSTPRALELRGQSQEQS

SEQ ID NO: 29 - DNA, 1785 bp, Mus musculus
  CDS (75)..(620)
ccacgcgtccgcccacgcgtccggcagccgccgcggaggtctttggccagtatcgattccacctgtccacaaacatggggaatatctttgcaaacctcttcaagggcctttttggcaaaaaagaaatgcgcattctcatggtgggcctggatgctgcagggaagacaacaattctatacaaacttaagctgggcgaaattgtgaccaccattcccaccattggtttcaatgtggagactgttgaatacaagaatatcagcttcaccgtgtgggatgtgggcggccaggacaagatccggccgctgtggcgccactacttccagaacacccaaggcttgatcttcgtagtggacagcaatgacagagagcgtgtgaacgaggcccgtgaagagctcatgaggatgctagctgaagatgagctccgagatgctgttctcttggtgtttgccaacaagcaggacctccccaatgccatgaatgcggccgaaatcacagacaagctggggctgcactctctacgccacaggaactggtacattcaggccacctgtgccaccagcggggacgggctctatgaaggactagattggctgtctaatcagctccggaaccagaagtgaaccagacccctccctccccctcacttcctcttctccgccctcagctttcctctcatgtggcaaacgtgcgactctgtggtcctgagtgccagaagctgtctccatgggttggtcacagtgtgcatcgcaccgtgctgtacatgtgcagacgcagcctgcagccaggtttttatttaatgtaaatagtttctgtttccactgaggcagtttctggtactcctatgcaatattacttagcttttttattgtaaaaagagaatcaactcaactgtcagtactgagaagggatttgggtgtaggggcacctggcctccgggagccattgggctgtagactggtgtcggtatccatttggtggttggtttttaacccaaactcagtgcattttttaaaaatagttaaaaatacaggacaagaacacttgaacacacagaacggagactatgcctagtgtaggttttgcagttaatggcctgaatgctagttatcagatcacctgtttcgctgtgggaacaggagagaaggtgatgaacaaaccaccatccgctgcatggtcacagtagagcccccgtgactcgcctgtctttgggtcacctgcattccatagcattgtgcttgtacttgtgctcacacggttacctagggtaggctgggagccattgtggggtgcagggcctggcttgtacttggtgtgtgcaaggcccaatggcagcctgcatacccagcctactcttgggcccacttggacgcgctggcaggaggcctgggtctcaccagcaggagtgcgtgcaaggtgggagggtcggtccattacagacccacatcctggagcacccccatctccatgtgtgaagtagcttcctccctcagcctgcaagggtccgatttgccatcgaaagacgacctctacttttttcttttgtattttgataaacactgaagaagctggagctgttaaatttatcttggggaaatctcagaactggtttatttggtgtcgtggaacctcttaccgctttcaatacacaattagtaatcaactgttttgtatacttgttttcagttttcatttcgacaagcactgtaattatagctgttagaataaagtctcttaactatttaaaaaaaaaaaaaaaaaaaaaaaaaaaaaaaaaaaaaaaaaaaa

SEQ ID NO: 30 - protein, 181 aa, Mus musculus
MGNIFANLFKGLFGKKEMRILMVGLDAAGKTTILYKLKLGEIVTTIPTIGFNVETVEYKNISFTVWDVGGQDKIRPLWRHYFQNTQGLIFVVDSNDRERVNEAREELMRMLAEDELRDAVLLVFANKQDLPNAMNAAEITDKLGLHSLRHRNWYIQATCATSGDGLYEGLDWLSNQLRNQK